(19) United States
(12) Patent Application Publication
(10) Pub. No.: US 2023/0324371 A1
(43) Pub. Date: Oct. 12, 2023

(54) EX VIVO BRAIN TUMOR MODEL

(71) Applicants: Paola ARLOTTA, Cambridge, MA (US); Mario L. SUVA, Boston, MA (US); Aviv REGEV, Hillsborough, CA (US); Kwanghun GHUNG, Lexington, MA (US); Vamsi MANGENA, Somerville, MA (US); Rafaela SARTORE, Cambridge, MA (US); President and Fellows of Harvard College, Cambridge, MA (US); The Broad Institute, Inc., Cambridge, MA (US); The General Hospital Corporation, Boston, MA (US); Massachusetts Institute of Technology, Cambridge, MA (US)

(72) Inventors: Paola Arlotta, Cambridge, MA (US); Mario L. Suva, Boston, MA (US); Aviv Regev, Hillsborough, CA (US); Kwanghun Chung, Lexington, MA (US); Vamsi Mangena, Somerville, MA (US); Rafaela Sartore, Cambridge, MA (US)

(21) Appl. No.: 18/021,156

(22) PCT Filed: Aug. 12, 2021
(86) PCT No.: PCT/US2021/045831

§ 371 Date: Feb. 13, 2023

Related U.S. Application Data
(60) Provisional application No. 63/064,905, filed on Aug. 12, 2020.

(51) Int. Cl. G01N 33/50; C12N 5/071

(52) U.S. Cl. CPC G01N 33/5082; G01N 33/5011; G01N 33/5023; C12N 5/0697; C12N 2513/00; C12N 2503/04

(57) ABSTRACT

Compositions and systems comprising a dorsal forebrain organoid having a core comprising less than 25% apoptotic or hypoxic cells and one or more tumor cells in the organoid.

### RELATED APPLICATION(S)

This application is related to and claims the benefit of U.S. Provisional Application No. 63/064,905, filed Aug. 12, 2020. The entire teachings of the application are incorporated herein by reference.

### TECHNICAL FIELD

The subject matter disclosed herein is generally directed to an in vitro or ex vivo tumor model and methods of using thereof.

### BACKGROUND

Due to the complex structure and biology of tumors, models for studying behavior and progression—and, in turn, therapeutic avenues—need to adequately recapitulate important features such as microenvironment, heterogeneity and inter-cellular communication within tumors. There is a need for in vitro tumor models that better capture the molecular and phenotypic spectrum of the corresponding tumor.

### SUMMARY

In one aspect, the present disclosure provides a composition comprising a dorsal forebrain organoid having a core comprising less than 25% apoptotic or hypoxic cells; and one or more tumor cells in the organoid. In some embodiments the tumor cells are glioma cells.

In some embodiments, the core comprises less than 20%, less than 15%, less than 10%, less than 5%, less than 1%, or less than 0.1% apoptotic or hypoxic cells. In some embodiments, the organoid has been cultured for at least 3 months. In some embodiments, the organoid comprises one or more of: corticofugal projection neurons, callosal projection neurons, cycling progenitors, immature corticofugal projection neurons, immature callosal projection neurons, immature projection neurons, immature interneurons, intermediate progenitor cells, outer radial glia, Cajal-Retzius neurons, and radial glia.

In some embodiments, the organoid comprises: about 17%-28% corticofugal projection neurons, about 40%-50% callosal projection neurons, about 4%-7% cycling progenitors, about 2% or less immature interneurons, about 3%-15% immature projection neurons, about 3%-6% intermediate progenitor cells, about 9%-14% radial glia, about 0.5% or less of Cajal-Retzius neurons, substantially no astroglia or cycling interneuron precursors, or any combination thereof.

In some embodiments, the organoid has been cultured for at least 6 months. In some embodiments, the organoid comprises one or more of: astroglia, callosal projection neurons, cycling progenitors, immature callosal projection neurons, immature interneurons, immature projection neurons, intermediate progenitor cells, outer radial glia, radial glia, ventral precursors, outer radial glia/astroglia, and cycling interneuron precursors. In some embodiments, the organoid comprises: about 6%-16% astroglia, about 7%-22% callosal projection neurons, about 5%-8% cycling progenitors, about 10%-31% immature interneurons, about 2%-10% immature projection neurons, about 1%-7% intermediate progenitor cells, about 22%-39% radial glia, about 4%-8% ventral precursors, substantially no corticofugal projection neurons or immature corticofugal projection neurons, or any combination thereof.

In some embodiments, the organoid has been cultured for at least 9 months or at least a year. In some embodiments, the organoid is a human dorsal forebrain organoid.

In some embodiments, the composition has a malignant/non-malignant cell percentage from 0 to 50%. In some embodiments the malignant cells are glioma cells. In some embodiments, the glioma cells originate from human patient-derived glioma cells implanted into the organoid. In some embodiments, the human patient-derived glioma cells comprise grade IV glioblastoma cells, high grade pediatric glioma cells, diffuse intrinsic pontine glioma (DIPG) cells, or isocitrate dehydrogenase (IDH) mutant glioma cells. In some embodiments, the human patient-derived glioma cells comprise IDH-wild type primary glioblastoma cells, IDH-mutant astrocytoma cells, or IDH-mutant oligodendroglioma cells. In some embodiments, the glioma cells comprise glioblastoma cells. In some embodiments, the glioma cells and/or cells in the organoid express one or more reporter genes.

In some embodiments, the glioma cells comprise one or more of: oligodendrocyte progenitor cell (OPC)-like, astrocyte (AC)-like, neural progenitor cell (NPC)-like, oligodendroglioma cell (OC)-like, or mesenchymal cell (MES)-like cells. In some embodiments, the glioma cells comprise two or more, or three or more of: OPC-like cells, AC-like cells, NPC-like cells, or MES-like cells.

In another aspect, the present disclosure provides a method of modeling glioma, the method comprising: implanting patient-derived glioma cells into a dorsal forebrain organoid with a core comprising less than 25% apoptotic or hypoxic cells. In some embodiments, the core comprises less than 20%, less than 15%, less than 10%, less than 5%, less than 1%, or less than 0.1% apoptotic or hypoxic cells. In some embodiments, the organoid has been cultured for at least 3 months. In some embodiments, the organoid comprises one or more of: corticofugal projection neurons, callosal projection neurons, cycling progenitors, immature corticofugal projection neurons, immature callosal projection neurons, immature projection neurons, immature interneurons, intermediate progenitor cells, outer radial glia, Cajal-Retzius neurons, and radial glia.

In some embodiments, the organoid comprises: about 17%-28% corticofugal projection neurons, about 40%-50% callosal projection neurons, about 4%-7% cycling progenitors, about 2% or less immature interneurons, about 3%-15% immature projection neurons, about 3%-6% intermediate progenitor cells, about 9%-14% radial glia, about 0.5% or less of Cajal-Retzius neurons, substantially no astroglia or cycling interneuron precursors, or any combination thereof.

In some embodiments, the organoid has been cultured for at least 6 months. In some embodiments, the organoid comprises one or more of: astroglia, callosal projection neurons, cycling progenitors, immature callosal projection neurons, immature interneurons, immature projection neurons, intermediate progenitor cells, outer radial glia, radial glia, ventral precursors, outer radial glia/astroglia, and cycling interneuron precursors. In some embodiments, the organoid comprises: about 6%-16% astroglia, about 7%-22% callosal projection neurons, about 5%-8% cycling progenitors, about 10%-31% immature interneurons, about 2%-10% immature projection neurons, about 1%-7% intermediate progenitor cells, about 22%-39% radial glia, about 4%-8% ventral precursors, substantially no corticofugal projection neurons or immature corticofugal projection neurons, or any combination thereof.

In some embodiments, the organoid has been cultured for at least 9 months or at least a year. In some embodiments, the patient-derived glioma cells grow to glioma cells comprising one or more of: OPC-like cells, AC-like cells, NPC-like cells, or MES-like cells. In some embodiments, the patient-derived glioma cells grow to glioma cells comprising two or more, or three or more of OPC-like cells, AC-like cells, NPC-like cells, and MES-like cells. In some embodiments, the patient-derived glioma cells comprise grade IV glioblastoma cells, high grade pediatric glioma cells, diffuse intrinsic pontine glioma (DIPG) cells, or isocitrate dehydrogenase (IDH) mutant glioma cells. In some embodiments, the implantation is performed by seeding the patient-derived glioma cells on a surface of the brain organoid. In some embodiments, the glioma cells comprise glioblastoma cells. In some embodiments, the method further comprises testing growth rates, transcriptional states, cellular lineages and hierarchies, cell morphologies, glioma-organoid microenvironmental interactions, invasive potential of glioma cells, intercellular communication, and/or intercellular connectivity of the glioma cells.

In another aspect, the present disclosure provides a method of identifying genetic variations related to glioma, the method comprising: introducing one or more genetic variations to the composition herein; and testing effects of the one or more genetic variations on growth rates, transcriptional states, cellular lineages and hierarchies, cell morphologies, glioma-organoid microenvironmental interactions, invasive potential of glioma cells, intercellular communication, and/or intercellular connectivity of the glioma cells.

In another aspect, the present disclosure provides a method of screening a therapeutic agent, the method comprising: contacting the composition herein with one or more candidate agents; and testing effects of the one or more candidate agents on growth rates, transcriptional states, cellular lineages and hierarchies, cell morphologies, glioma-organoid microenvironmental interactions, invasive potential of glioma cells, intercellular communication, and/or intercellular connectivity of the glioma cells. In some embodiments, the one more genetic variations is introduced to the glioma cells and the method comprises testing effect of the one or more genetic variations on cells in the organoid. In some embodiments, the one more genetic variations is introduced to cells in the organoid and the method comprises testing effect of the one or more genetic variations on the glioma cells.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows patient-derived glioblastoma cells (green, left panel) growing in human brain organoids (red, right panel) after 3 days of co-culture.

FIG. 2 demonstrates Diffuse Intrinsic Pontine Glioma (DIPG) cells showed signs of inter-cellular communications in brain organoids.

FIG. 3 shows patient-derived glioblastoma lines form interconnected cellular networks after 3 days of growth in human brain organoids. Images show a 100 micron z-stack taken using a confocal microscope.

FIG. 4 demonstrates that dissociated organoid and glioma cells (MGH143 and BT869) show high viability (CellTracker Live Stain Positive) after identical papain dissociation procedures.

FIG. 5 shows primary DIPG cells (BT869) infected with a GFP-expressing lentivirus growing in Neurosphere culture.

FIG. 6 shows GFP-tagged DIPG cells (green) growing in a human brain organoid (red, DAPI counterstain) after 11 days of co-culture.

FIG. 7 shows glioma cells in a brain organoid in an exemplary experiment.

FIG. 8 shows diverse exposure to environmental cues in an exemplary experiment.

FIG. 9 shows temporal dynamics of glioma growth in brain organoids suggested strong environmental influence.

FIG. 10 shows patient-derived glioma cells exhibited striking morphological heterogeneity in human brain organoids.

FIG. 11 shows transplant of an IDH1-R132H oligodendroglioma directly from a patient into a human brain organoid.

FIG. 12 shows healthy, GFP-tagged glioma cells were readily isolated from dissociated glioma-brain organoid co-cultures.

FIG. 13 shows the DIPG astrocyte-like signature (an exemplary experiment).

FIG. 14 shows the DIPG oligodendrocyte progenitor cell-like (shared) signature (an exemplary experiment).

FIG. 15 shows the DIPG cell cycle signature (an exemplary experiment).

FIG. 16 shows the DIPG oligodendrocyte progenitor cell-like (variable) signature (an exemplary experiment).

FIG. 17 shows the brain organoid microenvironment induced an OPC/OC-like to AC-like shift in patient-derived DIPG cells.

FIG. 18 shows cellular states represented in human GBM (mgh143) cells and an analogous human brain organoid model.

FIG. 19 shows gene signature scores of individual genes.

FIG. 20 shows hybrid states represented in human GBM (mgh143) cells and an analogous human brain organoid model.

FIG. 21 shows correlating scRNA-seq results with matched imaging readouts.

FIG. 22 shows an exemplary method for generating a glioma model and related organoid maturity and glioma model dependent cellular programs.

FIG. 23 shows an exemplary method for the identification of candidate targets for inhibiting glioma infiltration.

FIG. 24 shows an example of infiltration target (MDK).

FIG. 25 shows another example of infiltration target (DDR1).

FIG. 26 shows candidate DIPG infiltration targets (adhesion molecules).

FIG. 27 shows that adhesion molecules were upregulated in an organoid model coordinately map to the AC-state of the human tumor (BCH869).

FIG. 28 shows the result of FIG. 27 with AC gene removed.

FIG. 29 shows recreation of only 1 human glioblastoma (GBM) state using gliomaspheres.

FIG. 30 shows recreation of at least 3 human GMB states using the organoid glioma model.

FIG. 31 shows recreation of all 4 human GMB states using patient-derived glioma (PDX) cells.

FIG. 32 shows malignant cell scores across models for 8 gene signatures observed in human glioblastomas.

FIG. 33 shows tumor spheroids infiltrate human brain organoids. MDA-MB-231 GFP+ tumor spheroids co-cultured with 30 day old human brain organoids (dpf=days post fusion). Time lapse images obtained with stereo-microscopy.

FIG. 34 shows cancer cell colonization within the human brain organoid microenvironment. IF analysis with 100 micron thick slices of tumor-spheroid/organoid co-cultures. Single infiltrating MDA-MB-231 GFP+ cells show heterogeneous proliferative capacity and cellular morphologies in the human brain organoid microenvironment.

FIG. 35 shows that GFP+ cells that are isolated from a brain organoid and MGH143 (glioblastoma cell line) co-culture include both malignant and non-malignant cells. This effect is independent of the age of the brain organoid.

FIG. 36 shows that GFP-tagged MGH143 cells demonstrate evidence of projections as well as extracellular vesicle structures within the brain organoid.

FIG. 37 shows that GFP transcript reads can be mapped from the single cell transcriptomes of malignant and non-malignant cells that were obtained from a purification of GFP positive cells. This provides further evidence that the non-malignant cells received GFP transcript, and that their capture was not a technical error. Notably, there is a quantitative relationship between the number of GFP transcript reads in the malignant cells and the non-malignant cells that is suggestive of a dilutive process where GFP is transferred from malignant to non-malignant cells.

FIG. 38 shows that GFP+ positive cells that are captured can be clearly separated from the negative control; that is, the blank organoid without any implanted GFP+ glioma cells. This provides further evidence that capturing GFP+ non-glioma cells is not a technical error.

FIG. 39 shows a repeat of the chromosome number variation (CNV) analysis using bona-fide brain organoid cells as a reference for the inferCNV algorithm. This provides evidence that the captured GFP+ cells are in fact bona-fide non-malignant (or brain organoid) cells since they have the same CNV signature.

FIG. 40 shows that GFP+ non-malignant cells cluster tightly with GFP− brain organoid cells. Thus, in gene expression space, the GFP+ non-malignant cells captured from the model closely approximate what we know to be true brain organoid cells.

FIG. 41 shows that GFP transfer occurs indiscriminately to all cells in the human brain organoid. That is, of all the cell types observed in the human brain organoid, there is representation of GFP+ and GFP− non-malignant cells for all of them.

FIG. 42 shows methodology for collecting and profiling malignant and non-malignant cells from 5 different glioma models.

FIG. 43 shows that the GFP transfer phenotype and clustering, as described for MGH143 cells, applies to 2 more glioma models.

FIG. 44 shows that the CNV findings from GFP− and GFP+ cells are preserved across three separate glioma models.

FIG. 45 shows that malignant MGH143 cells extracted from a brain organoid model cluster into cell populations that map to NPC-like, MES-like, AC-like, OPC-like, and cell cycle programs observed in glioblastoma patient populations.

FIG. 46 shows that malignant MGG23 cells extracted from a brain organoid model cluster into cell populations that map to NPC-like, MES-like, AC-like, OPC-like, and cell cycle programs observed in glioblastoma patient populations.

FIG. 47 shows that malignant MGG101 cells extracted from a brain organoid model cluster into cell populations that weakly map to the NPC-like, MES-like, AC-like, OPC-like, and cell cycle programs observed in glioblastoma patient populations.

FIG. 48 shows that malignant cells from three different glioma models differentially map to the patient glioblastoma programs when in gliomasphere culture versus in a human brain organoid (method: Jaccard intersection between marker genes).

FIG. 49 shows that malignant cells from three different glioma models differentially map to the patient glioblastoma programs when in gliomasphere culture versus in a human brain organoid (method: gene correlation).

FIG. 50 shows that for a single glioma model, different microenvironments (including the patient) induce the canonical glioblastoma patient cell states to differing degrees, with the human brain organoid model exceeding other models for several states.

FIG. 51 shows that when GFP+ and GFP− brain organoid (non-malignant cells) are compared, differentially regulated genes can be identified that ostensibly relate to how glioma cells condition the surrounding microenvironment. The identified genes are related to endo/exo-cytosis and other adhesion related molecules.

FIG. 52 shows that the GFP transfer phenotype may be, in part, dependent on electrical activity, as there is less GFP+ cells represented after treating the organoid/glioma co-culture with TTX (an action potential blocker).

FIG. 53 shows that the glioma/brain organoid co-cultures can be treated with an electrical activity blocker without significant cell death.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment,” “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides for in vitro and ex vivo tumor models that capture the molecular and phenotypic spectrum of the corresponding tumor, e.g., important features such as microenvironment, heterogeneity and inter-cellular communication within tumors. Such models allow for reliable disease modeling and therapeutic testing at large scale and spatiotemporal resolution. In general, the models comprise an organoid and one or more tumor cells implanted into the organoid. For example, glioma cells, e.g., patient-derived gliomas cells, may be implanted into and grow in brain organoid models for further study and screening.

In one aspect, the present disclosure provides a composition or system comprising a brain organoid and one or more glioma cells. The glioma cells may be an established glioma cell line or patient-derived tumor cells. Patient-derived tumor cells may comprise cells directly isolated from a subject with a glioma, or cells obtained from a subject with a glioma and expanded in in vitro cell culture prior to being established in the brain organoid models disclosed herein. In some examples, the composition or system may comprise glioma cells of one or more types, e.g., OPC-like cells, AC-like cells, NPC-like cells, OC-like cells, or MES-like cells. When the glioma cells are implanted, the organoid may be at a certain age, e.g., have been cultured for a certain time, e.g., 3 months, 6 months or longer, to model a microenvironment in patients.

In another aspect, the present disclosure provides a method of modeling tumors using an in vitro or ex vivo model, the method comprising introducing one or more tumor cells, e.g., glioma cells (e.g., patient-derived tumor cells), into a brain organoid. In some other aspects, the present disclosure provides methods of using such tumor models, e.g., in identifying genes or other characteristics of the tumor being modeled, or in screening therapeutic agents in treating the tumor.

### Compositions

In one aspect, the present disclosure provides compositions and systems comprising in vitro or ex vivo tumor models. In general, the compositions and systems may comprise an organoid and one or more tumor cells implanted in the organoid. In some embodiments, the organoid is a brain organoid, e.g., a dorsal forebrain organoid. The tumor cells may be brain tumor cells, e.g., glioma cells.

In some embodiments, the composition may comprise malignant cells and non-malignant cells. In some embodiments, the composition may comprise malignant cells and non-malignant cells at varying ratios in order to model different tumor spatial or microenvironmental patterns (e.g., infiltrative edge vs. packed core). For example, the composition may have a malignant cell percentage of from 0 to 99%, from 0 to 90%, from 0 to 80%, from 0 to 70%, from 0 to 60%, from 0 to 50%, from 0 and 40%, from 0 to 30%, from 0 and 20%, from 0 to 10%, from 5% to 15%, from 10% to 20%, from 15% to 25%, from 20% to 30%, from 25% to 35%, from 30% to 40%, from 35% to 45%, from 40% to 50%, from 45% to 55%, or from 50% to 60%; and a non-malignant cell percentage from 0 to 99%, from 0 to 90%, from 0 to 80%, from 0 to 70%, from 0 to 60%, from 0 to 50%, from 0 and 40%, from 0 to 30%, from 0 and 20%, from 0 to 10%, from 5% to 15%, from 10% to 20%, from 15% to 25%, from 20% to 30%, from 25% to 35%, from 30% to 40%, from 35% to 45%, from 40% to 50%, from 45% to 55%, or from 50% to 60%.

The composition (e.g., when the organoid is a brain organoid) may have electrical activity. Electrical activity includes the transmission and/or reception of electrical signals, the transmission and/or reception of action potentials, and the change in charge generated (e.g., in individual nerve cells). The electrical activity may be measured using one or more electrodes, configured to measure variations of electric fields indicative of an activity of specific neural networks in the brain. The measurement may be facilitated by utilizing an EEG device/system. In some cases, the measurement may be facilitated by measuring an electrical activity of neural structures in the brain in response to a stimulation, e.g., such as an electrical stimulation or electromagnetic stimuli. In some cases, a first electrode and a second electrode may be placed in a region in the composition or system, such that an electrical signal passing through region via the first electrode and the second electrode reach the region.

### Organoid

The composition or system herein may comprise one or more organoids. An organoid may be a three-dimensional assembly that contains multiple cell types, arranged similarly to the cells in a specific tissue, and replicate aspects of the in vivo microenvironment and anatomy compared to a standard tissue culture model. The organoid may be capable of self-renewal and self-organization and exhibit similar organ functionality as the tissue of origin.

In some embodiments, organoids may be derived from stem cells (e.g., embryonic stem cells, induced pluripotent stem cells, etc.). Examples of organoids include cerebral organoids, thyroid organoids, intestinal organoids, testicular organoids, hepatic organoids, pancreatic organoids, gastric organoids, epithelial organoids, lung organoids, kidney organoids, retina organoids, inner ear organoids, and pituitary organoids.

In some examples, the organoid may be a brain organoid. A brain organoid may be an organoid that has anatomical and functional features that resemble brain function or function of a particular area of the brain. The brain organoid may include synthesized tissues that contain several types of nerve cells and other types of cells. In some embodiments, a brain organoid comprises one or more of: subpopulations of neurons and progenitors of the cerebral cortex (e.g., neuronal genes, interneurons, glia cells, forebrain cells, hindbrain cells, midbrain cells, forebrain excitatory neurons, corticofugal projection neurons, callosal projection neurons, TH+ neurons in neural network circuits, and the like), as well as retinal cell types (e.g., cortical neurons, subcortical neurons, sensory cells, Muller glial cells, canonical pigmented epithelial cells, photoreceptors, retinal ganglion cells, bipolar cells, amacrine cells, and the like).

Brain organoids can be produced using progenitor cells such as human pluripotent stem cells (hPSCs). The general methodology for producing cerebral organoids includes culturing the stem cells under conditions suitable for the development of an embryoid body. The cell culture may then be induced to form a neuroectoderm, and the neuroectoderm is grown in a protein matrix. The neuroectoderm may begin to proliferate and grow and may be transferred to a tissue culture vessel where the cerebral organoids will continue to develop. Brain organoids may differentiate into one or more of various neural tissue types, such as the optic cup, hippocampus, ventral parts of the telencephalon and dorsal cortex.

### Dorsal Forebrain Organoids

In some embodiments, the brain organoid is a dorsal forebrain organoid (DFO). A DFO may have anatomical and functional features that resemble the dorsal forebrain. In some embodiments, the DFO comprises cells expressing one or more dorsal forebrain markers, dorsal forebrain progenitor markers, early pan-neuronal markers, neuronal markers, and/or cortical markers. For example, the DFO comprises cells expressing one or more following markers: MAP2, EMX1, PAX6, CTIP2, SATB2, SOX2, Ki67, FOXG1, HOPX, TBR1, VGluT1, PSD95, and TBR2. The DFO may comprise cells that express at least 1, at least 2, at least 3, at least 4, at least 5, at least 6, at least 7, at least 8, at least 9, at least 10, at least 11, at least 12, or 13 of these markers. In some embodiments, the DFO comprises cells expressing MAP2 and PAX6 markers. In some embodiments, the DFO comprises cells expressing MAP2, PAX6, and EMX1 markers. In some embodiments, the DFO comprises cells expressing CTIP2 and SATB2 markers. In some embodiments, the DFO comprises cells expressing MAP2, PAX6, EMX1, CTIP2, and SATB2 markers. In some embodiments, the DFO expresses one, two, or all three of TBR2, Reelin, and TBR1. In some embodiments, the DFO expressing the noted markers has been cultured for at least one month, at least three months, at least six months, at least 9 months, at least a year, or longer.

In some embodiments, the DFO has a core. In some embodiments, the core comprises the cells of the DFO that are at least 100 μm, at least 125 μm, at least 150 μm, at least 175 μm, at least 200 μm, at least 225 μm, or at least 250 μm from an exterior surface of the DFO.

In the DFO herein, there may be very low apoptosis or hypoxia in cells in the core. In some examples, apoptosis and hypoxia in cells may be measured using the mSigDB hallmark gene set for apoptosis or hypoxia, by detecting CASP3 (e.g., via immunohistochemistry), or using immunohistochemistry for relevant apoptosis or hypoxia markers.

In some cases, the core may comprise less than 30%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.05%, or less than 0.01% apoptotic or hypoxic cells.

In some cases, the organoid may comprise less than 50%, less than 45%, less than 40%, less than 35%, less than 30%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.05%, or less than 0.01% apoptotic or hypoxic cells.

### Age of Organoids

In some embodiments, when the glioma cell(s) is implanted to the organoid, the organoid has been cultured for a period of time. For example, the organoid (e.g., the DFO) may have been cultured for at least 1 month, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. In some examples, the organoid has been cultured for 3 or more months and its core comprises less than 20%, less than 15%, less than 10%, less than 5%, less than 1%, or less than 0.1% apoptotic or hypoxic cells. In some examples, the organoid has been cultured for 6 or more months and its core comprises less than 20%, less than 15%, less than 10%, less than 5%, less than 1%, or less than 0.1% apoptotic or hypoxic cells. In some examples, the organoid has been cultured for 9 or more months and its core comprises less than 20%, less than 15%, less than 10%, less than 5%, less than 1%, or less than 0.1% apoptotic or hypoxic cells. In some examples, the organoid has been cultured for a year or longer and its core comprises less than 20%, less than 15%, less than 10%, less than 5%, less than 1%, or less than 0.1% apoptotic or hypoxic cells.

In some embodiments, the organoid has been cultured for about 1 month to about 3 months, and the organoid comprises one or more of: 17%-28% corticofugal projection neurons, about 40%-50% callosal projection neurons, about 4%-7% cycling progenitors, about 2% or less (including 0%) immature interneurons, about 3%-15% immature projection neurons, about 3%-6% intermediate progenitor cells, about 9%-14% radial glia, and about 0.5% or less (including 0%) of Cajal-Retzius neurons.

In some embodiments, the organoid has been cultured for at least 3 months. In some cases, such organoids comprise one or more of: corticofugal projection neurons, callosal projection neurons, cycling progenitors, immature corticofugal projection neurons, immature callosal projection neurons, immature projection neurons, immature interneurons, intermediate progenitor cells, outer radial glia, Cajal-Retzius neurons, and radial glia. In some examples, the organoid comprise about 17%-28% corticofugal projection neurons, about 40%-50% callosal projection neurons, about 4%-7% cycling progenitors, about 2% or less immature interneurons, about 3%-15% immature projection neurons, about 3%-6% intermediate progenitor cells, about 9%-14% radial glia, about 0.5% or less of Cajal-Retzius neurons, or any combination thereof. In some cases, the organoid comprises substantially no astroglia or cycling interneuron precursors.

In some embodiments, an immature projection neuron in an organoid cultured for at least 3 months is characterized as an organoid cell that overexpresses, as compared to the rest of the organoid cells, at least 5, 10, 20, 30, 40, 50, 75, or all 85 of the following genes: BASP1, TUBB2B, MAP1B, TUBA1A, MLLT11, PCSK1N, PGK1, GAP43, CRMP1, HILPDA, CD24, ARMCX3, TAGLN3, NRN1, MARCKS, UCHL1, GSTA4, ENO2, STMN4, HMP19, TMSB15A, APP, TMEM132A, NCAM1, HES4, NCALD, GPR162, RUNX1T1, RCN1, INA, GPC2, EGR1, KCNQ1OT1, FAM213A, DNER, NEFL, MYL6, CADM3, SCG2, MIAT, CLU, NDN, ATF3, TM7SF2, CHGA, LRRN3, CXXC5, ETFB, SYP, KLC1, LDHA, RCN2, SCG5, CHD4, GNG3, ID4, ANK3, CNTNAP2, ARMCX1, NOVA1, APLP1, ARID5B, RNF5, LGALS3BP, MAP6, CA11, INSM1, CELF4, TMEM14C, OLFM1, FAM57B, CITED2, HACD3, BLCAP, ISYNA1, LSAMP, MDK, SYT5, AP1S2, RSRC1, BSDC1, DUT, SLF1, SEMA6A, and CHD7.

In some embodiments, an immature callosal projection neuron in an organoid cultured for at least 3 months is characterized as an organoid cell that overexpresses, as compared to the rest of the organoid cells, at least 5, 10, 20, 30, 40, 50, or all 55 of the following genes: SOX11, SLA, CLMP, ARHGAP21, TCF4, MT-ND3, GADD45G, FNBP1L, MEIS2, DCX, NFIB, MIAT, CADM2, ARL4C, MN1, DDAH2, LINC01102, TPGS2, CHD3, RND3, TTC28, MEX3B, DNER, GSE1, C14orf132, DPYSL4, NEDD4L, FAM60A, NUP93, RERE, SERINC5, TMSB15A, AUTS2, STARD4-AS1, MUM1, LIMD2, PHLDA1, FLRT2, KCNQ2, SERP2, SUN2, PLXNA4, ZNF300, RNF182, LRRC7, ZNF195, BAZ2B, PLPPR5, HS3ST1, ACOT7, INHBA, ZNF627, EPHA4, CAMK2B, and INSM1.

In some embodiments, a callosal projection neuron in an organoid cultured for at least 3 months is characterized as an organoid cell that overexpresses, as compared to the rest of the organoid cells, at least 5, 10, 20, 30, 40, 50, 75, 100, 125, 150, 175, 200, or all 237 of the following genes: EXOC4, GPR85, STMN2, INHBA, RNF182, NELL2, NEUROD6, SATB2, MEF2C, NHSL1, SNX7, SERPINI1, NREP, NCALD, NEUROD2, CAMKV, BHLHE22, DCX, DACT1, HSPA8, BASP1, MCUR1, CD24, FABP7, RTN4, FAM49A, NEFM, RAB3A, PLXNA4, INA, OLFM1, PTPN2, MT-CO2, MAP1B, GNAI1, MN1, DEAF1, PRKACB, MT-ATP6, PKIA, PEBP1, NSG1, NCAM1, SRGAP1, MAPT, RASL11B, SHTN1, ZEB2, FAT3, TUBA1A, RAC3, ATAT1, DSTN, TMEM14A, JAKMIP1, RBFOX2, CRMP1, LRRC7, PPFIA2, ATP1A3, ST3GAL1, SLC8A1, MYT1L, CSRNP3, STMN4, TSPO, SCD5, SQLE, PAK7, CAMK2B, ATP2B1, ADCY1, COTL1, MT-CYB, SYBU, NUDT3, CSRP2, GFOD1, ELAVL3, TMEM160, HMGCS1, PIK3R1, AKAP7, CHCHD6, MPPED1, CDK5R1, AP3S1, GDAP1L1, DPYSL3, BCL7A, DNER, GNG3, DUSP23, APLP1, MEAF6, NAV1, PTPRD, ANK2, ANKRD46, SBK1, MMD, PHACTR3, NME1, BOP1, ADD2, MAP4, CTXN1, GNAO1, C20orf27, RAP1GDS1, HS3ST1, SH3GL3, STARD4-AS1, NOL4, SPTAN1, TMEM35, PCLO, SMAP2, AMN1, CELF3, MAP4K4, SSBP4, C2orf80, TBC1D14, RBFOX1, CHGB, PARP6, STRBP, RGS17, GRIN2B, KLHL8, ATP1B1, JPH4, SERP2, FKBP1A, MYCBP2, HMGCR, EML1, MT-ND5, PLPPR5, FARP1, FLRT2, PGD, LRRN3, NEO1, ACTN2, ATP6V0E2, FOXP1, ACAT2, CELF1, DAB1, MAPRE3, SPIN1, RRM2B, LDB2, TUSC3, ZWILCH, FAM84A, SV2A, PWAR6, ODF2L, PRKCZ, CMIP, PPP1R14C, RUNDC3B, FSD1, PSD3, ELOVL6, PAK1, RUNDC3A, CACNG8, SRD5A1, GRIA1, RP11-490M8.1, NPB, RNF219, TUBB4A, NLRP1, SSX2IP, HIVEP2, RP11-660L16.2, HSD11B1L, GFOD2, AFF3, SEC61A2, JAKMIP2, UBE2E3, BEX5, SYT5, TSPYL1, ARHGEF9, MAPRE2, PTPRO, FASN, GNAZ, HOMER1, STC1, FAM127A, RUNX1T1, DYRK2, BIVM, FBXL2, PSD, ELMO1, ATP9A, DLG2, LINC01503, TCEAL7, TMEM150C, SCG2, SNN, BOLA3-AS1, HEBP2, MGLL, ARHGAP33, MT-ND4L, CCDC184, DDX25, MYO5A, CCSAP, BAD, RASGRP2, FBXL15, BRINP1, LYPD1, SNX32, KATNB1, MASP1, ROGDI, DACH2, B4GALNT1, TCEAL1, RPRM, PDE4DIP, PGP, ULK3, and CHN2.

In some embodiments, an immature corticofugal projection neuron in an organoid cultured for at least 3 months is characterized as an organoid cell that overexpresses, as compared to the rest of the organoid cells, at least about at least 5, 10, 20, 30, 40, 50, 75, 100, 125, 150, 175, 200, 250, 300, 350, 400 or all 416 of the following genes: CTA-29F11.1, RNF165, ILF3-AS1, ZNF436-AS1, RP11-51J9.5, IER5, PRR7, BRD2, ATP6V0B, RP11-356J5.12, GPR22, RP1-39G22.7, MLLT4-AS1, RRAGA, EID2B, RP4-798A10.7, BBC3, RP11-352M15.2, NAA38, VAMP2, RFK, GABPB1-AS1, NCBP2-AS2, NSMCE3, PDRG1, FBXL15, RP11-395G23.3, TAF7, POP7, HIST3H2A, TMA7, SNHG7, ZNF830, RP11-1094M14.11, IMP3, SPINT2, H1F0, BLOC1S4, MAPKAPK5-AS1, SAC3D1, MESP1, KCNQ1OT1, LINC00526, SNHG15, TRMT10C, HIST1H1C, EPC1, PHLDA3, FBXW7, PSMG3, CSTF3, EPM2AIP1, PET117, EPB41L4A-AS1, C16orf91, LINC00685, AMD1, NEFL, MAGEH1, AC093323.3, TXNIP, KBTBD7, MOAP1, MED19, BLOC1S2, EFNA3, MRPL34, PCF11, RAB33A, RP11-410L14.2, C19orf53, RP11-660L16.2, NAP1L3, C19orf25, C9orf78, NR2F6, NGDN, RP11-792A8.4, MRPL44, CHD2, PPID, ARPC5L, GSPT2, TUSC2, CAMLG, PEX13, ACYP1, POLR1C, DLL3, CDKN2AIPNL, UQCC3, DGUOK, F12, TBCC, C15orf61, PFDN2, ATG101, SLC16A1-AS1, SCNM1, LINC01315, SCOC, SLBP, TRIM32, EMC6, TAF9, TSC22D3, MRFAP1L1, TCEAL5, NPM3, LINC01006, ANKRD54, LINC01560, SELM, ZNF821, NUDC, IMP4, DOHH, RGS2, ALDOA, INTS6, C11orf71, ZSCAN16-AS1, RNF113A, HIST1H2BG, PITHD1, NFKBIA, COX17, IMMP1L, ERV3-1, CHAMP1, DDX24, CYCS, TMEM11, FAM103A1, PRKAG2-AS1, TMEM251, TYW5, PPFIA3, BOLA3-AS1, TIMM17A, FEM1A, RBM4, HIRIP3, SRSF8, LINC00662, PLK3, ZCCHC7, RDH14, ATP5G1, EIF4A2, MAGEF1, OAT, DACH2, RRS1, CCDC184, TIGD1, ASB8, CDKN2D, THAP2, UTP3, C6orf120, ZNF622, IP6K2, THAP9-AS1, EIF2B2, TM2D3, ATXN3, NDUFAF4, ZNF281, WDR74, MRPL32, CNOT8, RASL10A, PPP1R8, MKRN1, DPM3, ANKRA2, KBTBD6, PTS, SNHG8, RNASEH1, CHMP1B, GLRX5, SPIN2B, PRRT1, RCHY1, CTSL, SNAP47, CFAP20, MPHOSPH10, BOLA1, LARP6, PAK1IP1, TIPRL, TRAPPC4, ZFAS1, TMED9, HIST2H2BE, ZNF574, FAM110A, WBP5, PPP4R2, NRBF2, AHSA1, C12orf73, RP9, NUDCD2, THAP11, C2orf69, C1orf35, CCDC115, LYPLA2, ALAS1, RP11-83A24.2, TMEM167B, THAP5, LINC00667, PELO, GTF2B, TSPYL2, MEDT, PCYOX1, CNPPD1, SNX10, CSGALNACT2, GRPEL1, ING2, FUT11, PRPF4, RBM22, PPP1R2, SURF6, WBP11, SURF2, THAP3, TAF12, MED6, ZBTB43, KIAA0907, RANBP6, SAMD8, SS18L2, SDHAF1, LINC01003, C17orf58, CDKN2AIP, DUSP12, ZNF791, SDHAF2, TMEM55B, TMUB1, MAD2L1BP, BEX5, TAF1D, CCDC51, ZFPL1, ARRDC3, PDK1, CBR1, CDC37L1, MPHOSPH8, ELOVL4, PRPF38A, PPM1A, ZNF397, DAXX, ADPRHL2, ING1, MMADHC, EBP, METTL2A, RPA2, DUSP18, MRPL10, TOPORS, MAP9, G3BP2, FUCA1, MRPL49, CMBL, SIKE1, TMEM87A, TMEM183A, FKBP7, CEP57, AAR2, NXT1, RNF41, RASSF1, ATP6V1G2, PNRC2, BAG5, SCO1, DNTTIP2, RBM4B, SIRT6, CITED2, SLC39A1, CLN5, MRPS14, CWC25, LRRC59, NABP2, FDFT1, DDX21, TTC9C, P4HB, TMEM205, GGNBP2, TMEM199, CCND3, TMEM70, SCAMP3, FTSJ2, ZNF667-AS1, PARP2, ZNF131, DIS3, YIPF4, EIF2B5, PI4KB, STIM2, LETMD1, THUMPD1, HIST1H2AC, RNF4, CLK4, ZNF274, SIRT7, CDK19, KANSL2, SEC11C, CEBPZ, NECAP1, CLK1, ZCCHC10, EED, GSKIP, FRG1, CSTF1, CCDC130, TAF13, ZMAT3, CDC40, PDCD2L, TCTN3, DEXI, C1orf174, AKT1S1, PIM3, GOT1, RNF13, C1orf109, ELP5, BRIX1, SLC35A4, RIOK2, RPL39L, FEN1, FEM1B, ZNF430, DYRK4, NKIRAS2, ELOVL6, EBLN3, ANKRD49, TMED3, GORASP2, NBR1, POLE3, PREB, DEDD2, USP15, SUN1, TRAPPC2P1, NUP50, FAM126B, CTDSPL2, C22orf29, MTHFD2, NOLC1, YIPF5, OSER1, MUL1, HSD17B7, CCDC174, VCPKMT, PDP1, AKAP17A, DNAJB4, RGS16, GEMIN2, CRIPT, CXXC1, CCP110, GPN3, RAB39B, RBBP5, ZNF581, C1orf131, BNIP1, CXorf40B, ZNF331, TNRC6C, RPP30, PRKAB1, RFC4, GAR1, ARID3A, ANKRD37, TMEM136, PIM1, PNO1, MYNN, MPPE1, and UTP6.

In some embodiments, a corticofugal projection neuron in an organoid cultured for at least 3 months is characterized as an organoid cell that overexpresses, as compared to the rest of the organoid cells, at least 5, 10, 20, 30, 40, 50, 75, 100, 125, 150, 175, 200, 250, or all 273 of the following genes: KAZN, PDE1A, GPR22, ETV1, FEZF2, IGSF21, BRINP1, TLE4, CELF4, SNAP25, CTNND2, SYT1, SCD5, SSBP2, OLFM1, NELL2, CXADR, MAP1B, MAPT, NBEA, VAMP2, RALYL, GRIA2, SPINT2, HMGCS1, NFIA, CPE, VRK1, PBX1, NEUROD2, RBFOX2, RBFOX1, DYNC1I1, LINC00461, SQLE, LINGO1, AGAP1, NFIB, DOK6, RP11-356J5.12, SLC26A4-AS1, KIDINS220, RTN4, PPP3CB, PPP3CA, SEZ6, INA, SESN3, CLSTN1, ITSN1, PNMA1, TMEM108, RFK, PHACTR3, DPP6, FKBP1B, PRKACB, SHTN1, NLGN1, CCDC107, NDN, MSRA, TMEM35, NSMF, TUSC3, JAKMIP2, APP, SULT4A1, FXYD6, RGS17, GNAO1, EFNA3, ANKS1B, H1F0, GABPB1-AS1, SCAMP1, NETO2, RP11-660L16.2, IER5, DCLK1, BCL11A, KIF3A, RAB33A, ITFG1, DEAF1, RPRM, NTRK3, DSEL, REEP1, H2AFJ, NFIX, ENOPH1, PRR7, NCAM1, SRRM4, ANKMY2, SCAI, WIPF3, DACH2, PHYHIP, RASL10A, DUSP5, PSD3, STT3B, ARL6IP5, GALNT11, ARL4D, CAMK2G, KCNQ1OT1, F12, SESTD1, RP11-25K19.1, HK1, FDFT1, TNIK, CMB9-22P13.1, JAKMIP1, TMEM132A, IDS, ENO2, SH3KBP1, KIFAP3, BZW2, NOV, CCDC184, CEP19, THSD7A, KCTD13, MOAP1, GTF2H5, CAMK1, SLC25A4, TMEM63B, IDH3G, CADPS, PRNP, C14orf1, DKK3, CDC40, DBP, FABP5, ALAS1, CADM1, STXBP1, LINC00685, CELF5, MYCBP2, LINC00632, PSMD1, WAC-AS1, ARL2, MT-ND6, PPID, CITED2, PNMAL1, IDH1, DAB1, ING2, THOC3, TRIB1, ROGDI, FASN, PICALM, ABR, SEMA3A, ACAP1, POLR3GL, LGALSL, PFKFB3, MESDC1, ACLY, ATP13A2, RP11-511P7.5, POMGNT2, POLD2, SLC16A1-AS1, TIPRL, BOLA3-AS1, FARSB, RP1-39G22.7, INTS12, ELOVL6, SEC61A2, GRIA3, PIGP, CYFIP2, GAL3ST3, THAP5, MYO5A, NFASC, RNF41, DNAJC12, CRK, TRIM32, RP11-686O6.2, DUSP18, RUVBL1, IGF2BP2, PITHD1, PDHA1, AC093323.3, PGK1, OXCT1, ENHO, KIFC2, PCSK7, SDCCAG8, TMEM106B, FGF13, GNB5, THAP1, COQ7, SCAPER, CA11, CDK19, G2E3, MCTS1, LINC00936, GSK3B, TRIM9, GPR137, AP001372.2, MAP1A, PPCS, POLR3K, GFOD2, RAD50, ING1, PCAT6, PRPSAP2, EID2B, RP11-127B20.2, TMEM121, ACOT13, CYB5D2, C6orf136, LINC00094, PTDSS1, DZIP3, CTC-241N9.1, HDHD3, TAF12, EPB41L3, CCP110, ZNF529, EBLN3, PKIB, ARRDC1-AS1, RP11-83A24.2, PTPRF, TTPAL, IFT22, ADAL, TNNI3, LRRC49, TRAM1L1, ABHD8, PAXIP1-AS1, FAM220A, ERRFI1, MECR, COQ3, STK16, MYLIP, KBTBD4, RP6-65G23.3, SPIN2B, RP11-115C21.2, TMEM5, TNIP1, RNASEH1-AS1, CUTC, and NIT2.

In some embodiments, an intermediate progenitor cell in an organoid cultured for at least 3 months is characterized as an organoid cell that overexpresses, as compared to the rest of the organoid cells, at least 5, 10, 20, 30, 40, 50, 75, 100, 125, 150, or all 167 of the following genes: NFIA, PRDX1, MARCKS, SOX4, CALD1, CORO1C, HMGN2, Clorf16, SSTR2, TMEM123, PAX6, CMC1, UBE2E3, EEF1D, SOX11, SYNE2, EZR, H3F3B, RPS6, ZBTB20, HLA-A, RCN2, AP1S2, NAP1L1, PHLDA1, B2M, MEIS2, TMEM98, PGAP1, MDK, SRSF6, TFDP2, ITGB1, MYO6, HPCAL1, NKAIN3, ROBO2, KCNQ2, GLTSCR2, SORBS2, LYPD1, BAZ2B, ADGRG1, CCND2, MDFI, MPST, CXXC5, RND3, STK17A, GADD45G, NR2F1, TCF4, MRPL42, HDAC9, MSI1, GOLIM4, RBPJ, FZD3, POU3F2, SPAG9, PGRMC2, RPS27L, BBX, HLA-B, DECR1, PRKX, MLLT4, BICD1, EBPL, USP3, HLA-C, BTG1, PHYHIPL, MSI2, TMX1, NME4, H2AFV, ASCL1, PNRC1, FYN, ATP6V0E1, BTG2, TANK, FEM1C, SKA2, FAM60A, NRN1, SEPT9, PDXK, CNN2, JAM2, PNKD, TBL1XR1, DBN1, CDK4, PNRC2, FBLN1, PTTG1IP, BAZ1A, DHRS7, KDM1A, DSEL, REC8, IFI27L2, SERINC2, C14orf132, EHBP1, DNAJC4, EZH2, LIMD2, GLUL, SMARCA5, NUDT5, GCA, USP47, RAB13, LEPROT, NFIC, LIMS1, CBFA2T2, AAMDC, CPLX2, ROCK1, AMOTL2, HADHB, LHX2, SETBP1, CHGA, TSPAN6, FOXN3, TMTC4, LMNB1, ACTL6A, POU3F3, CNR1, EMX2, RPA1, MARCH1, NDUFA7, CLIC1, BTG3, MESDC2, CLMP, ALDH7A1, TRIM24, ECI2, GNG4, HMG20B, LIMA1, TMPO, FUBP3, PAG1, SZRD1, ZFAND3, TLE3, LITAF, DAP, DDR1, PAM, FRMD4A, RIT1, MAPK10, STAT3, TECPR1, MEST, MIR124-2HG, and CNTNAP2.

In some embodiments, a radial glia in an organoid cultured for at least 3 months is characterized as an organoid cell that overexpresses, as compared to the rest of the organoid cells, at least 5, 10, 20, 30, 40, 50, 75, 100, 125, 150, 175, 200, or all 226 of the following genes: VIM, FTH1, BNIP3, FTL, GAPDH, ENO1, EIF1, CD9, SLC3A2, CLU, SOX2, DDIT3, NEAT1, RCN1, CD63, TCEA1, HSPB1, IGFBP2, MT2A, GADD45A, TGIF1, RPS27L, ALDOA, RPL41, SERPINH1, ANXA5, ADM, BCAN, RPL36, PHGDH, RPS20, SHMT2, PSAT1, SLC16A1, ZFP36L1, PGK1, CD99, P4HA1, SYPL1, SAT1, HSPA5, ATF4, RPS27, CXCR4, HES1, NFE2L2, CCNG1, SERPINE2, GNB2L1, SLC16A3, RGS16, HSD17B14, DARS, TPT1, RPL30, BLVRB, ATF3, SDCBP, FAM162A, HILPDA, TTYH1, EEF1D, DDIT4, PON2, SOX9, VEGFA, ATRAID, NPC2, SLC2A3, CD164, EMP3, PDLIM4, PNRC1, TMEM123, CANX, MT1X, RPL21, WSB1, LITAF, BTG3, HOPX, CTSD, GNG5, RP11-395G23.3, SCD, CRYAB, PGM1, DNAJC1, HADHB, QKI, ATP6V0E1, CSTB, GPT2, P4HB, BTG2, RHOC, CNN3, PAX6, BTG1, MID1IP1, TMEM47, XBP1, KCNG1, ID4, CALR, GPI, EMX2, NOV, PPT1, ST13, NT5C, HERPUD1, DNAJB9, ACADVL, PHYH, VKORC1, SPTSSA, ILK, MALAT1, SPG20, PRDX4, CEBPG, ADGRG1, EMD, CYR61, ITM2C, SRI, HLA-A, RPL22L1, ANKRD37, CIB1, TRIM9, B2M, HLA-B, TSC22D4, JAM2, MTHFD2, RPS16, PFKP, HLA-C, SSR3, GLUL, TMEM38B, ETV1, MIF, MYL12A, GBAS, CLNS1A, LMNA, EGLN3, PIM3, SNX2, ACAA2, CYBA, FERMT2, NGLY1, FOS, CNIH1, SNX5, FUBP3, CRYL1, SERF2, ALDH3A2, TAGLN2, GOLIM4, EPHX1, TSPAN6, TRAM1, SRA1, MESDC2, ACTN1, ETV5, ITGB1, TXNRD1, ZFAND3, AK2, PTTG1IP, CFAP36, SERP1, CHPT1, PDIA6, GCSH, ECI2, IRF2BP2, LDHA, BBX, PPIB, RHOA, RNF187, TMED7, SELK, SEPT2, LAPTM4B, ARL6IP6, CMTM6, PDIA4, EGR1, UBXN4, PAICS, CDK2AP2, C5orf28, PEX2, RAB13, RER1, ANP32B, GPX1, KDSR, TULP3, FAM84A, HBP1, FXR1, BAG3, GHITM, TMEM179B, RAB9A, SPNS1, DNPEP, RAP1A, TMEM230, TMEM263, MIF4GD, USO1, HIST1H1C, NHSL2, TMEM14C, ARRDC3, and TMX1.

In some embodiments, an outer radial glia in an organoid cultured for at least 3 months is characterized as an organoid cell that overexpresses, as compared to the rest of the organoid cells, at least 5, 10, 20, 30, 40, 50, 75, 100, 125, 150, 175, 200, 250, 300, or all 308 of the following genes: GFAP, ID3, HOPX, BCAN, PON2, SPARC, CLU, ID4, HES1, SOX2, PTN, ZFP36L1, TTYH1, SOX9, SCRG1, CST3, LRRC3B, DBI, RHOC, QKI, PEA15, DDAH1, SFRP1, VIM, HSPB1, ANXA5, C1orf6l, GPM6B, CNN3, SH3BGRL, HMGN3, B2M, FABP7, SRI, CD63, CKB, LIMA1, GNG5, NCAN, TAGLN2, CRYAB, LITAF, MT2A, PTPRZ1, SEPT9, PSAT1, GSTP1, PAX6, ITM2C, SEPT2, RCN1, SERF2, CD9, RPS27L, NDRG2, RHOA, ANXA6, EMP3, CYBA, PDLIM4, EZR, TSC22D4, SAT1, TMEM98, TGIF1, IFI6, GLUL, TMEM123, AP1S2, NME4, SYNE2, NFE2L2, MDK, MYL6, PHLDA1, DECR1, HADHB, CALD1, DNAJC1, NPC2, DKK3, PFN1, EEF1D, SDCBP, TMEM47, CAMTA1, ECI2, SPTSSA, Clorf122, RPS6, PPDPF, PSME1, POLR2L, CLIC1, SLC35F1, NT5C, DOK5, SEPT11, DNPH1, GPC4, MSI1, LINC00998, PDLIM7, TSPAN6, TSPAN3, SYPL1, HES4, RAB13, CCDC109B, H2AFV, PHGDH, MYL12A, SLC25A26, GBAS, ITGB1, PCBD1, SNX5, BAALC, C12orf75, PRDX6, AAMDC, PGM1, DHRS7, NKAIN3, PHYHIPL, ZBTB20, ID1, CRYL1, HMGN2, SLC25A6, MDFI, NDUFA11, ACAA2, TRIM9, HEY1, ABCD3, TMA7, TMEM132B, ADGRG1, OST4, FEZ2, CSTB, GOLIM4, ALDH7A1, FERMT2, BLOC1S1, NAP1L1, MAGED2, RDX, PXMP2, RCN2, PEX2, CD164, ATP6V0E1, CLNS1A, CXXC5, CDK4, C17orf89, ASPH, DDR1, PGLS, REEP3, ALDH9A1, KLHDC8A, HDDC2, DCXR, EFNB1, PTTG1, LHX2, C7orf50, FUBP3, EMX2, BTG3, NDUFA13, ARL6IP6, ADK, CNP, GOLM1, HIBCH, KTN1, GNAS, SEC11A, HMGN1, PSME2, HMG20B, MCL1, GPX1, KIAA0101, COMT, ACADVL, PTTG1IP, BBX, RP3-525N10.2, PHIP, SNX17, NUDT4, ROBO1, PLEKHO1, GCA, URM1, NUDT5, CD151, EGR1, HAT1, RNASEH2C, PPP1CA, UBE2E1, MGMT, CTNNBIP1, SCCPDH, POLR2J, ACTN1, APOA1BP, ILK, AKR7A2, PDIA6, ASCL1, TMEM230, PNKD, CHCHD10, TXNRD1, HADHA, LMNA, EIF2AK2, NME3, KLF6, ACADM, ETFA, CFL2, GPSM2, IDH2, JUNB, PDCD4, SMC4, NEAT1, PMF1, RHOBTB3, GADD45A, ANP32B, ABAT, HSD17B12, ZFAND3, CLDND1, TMBIM4, PEPD, TIMP2, RAB9A, DBNL, COMMD4, UQCC3, ROMO1, WDR1, TCF25, SESN3, COA4, NUTF2, UBXN4, MIF4GD, BLVRB, SNRPD3, MPPED2, C11orf31, MMP24-AS1, NRCAM, PAICS, AHCY, COPRS, SHISA4, ANGPTL4, CNTFR, PHYH, NFIC, PRCP, CTSD, WDR6, KLHL5, SMDT1, TLK1, NDRG4, GPT2, SMARCA1, ADSL, FKBP3, RNF130, CTSL, CTBP2, SRSF2, MRPL23, CYB5R3, HADH, PRSS23, REPS1, CNDP2, DGCR6L, ALDH3A2, JPX, SERINC2, LRRFIP1, REPIN1, AC004556.1, HYI, LTBP3, ENSA, EHBP1, LYPLAL1, MCM7, TYMS, and NASP.

In some embodiments, a cycling progenitor in an organoid cultured for at least 3 months is characterized as an organoid cell that overexpresses, as compared to the rest of the organoid cells, at least 5, 10, 20, 30, 40, 50, 75, 100, 125, 150, 175, 200, 250, 300, 350, 400, 450, or all 472 of the following genes: PTTG1, KIAA0101, HMGB2, SMC4, H2AFX, CKAP2, CENPW, CKS1B, CKS2, HMGN2, SOX2, TUBA1B, H2AFV, UBE2T, UBE2S, HMGB3, TUBB4B, HMGB1, CKB, HSPB1, PHGDH, HNRNPA2B1, KIF22, SFRP1, DHFR, HMGN3, PTN, KPNA2, PAX6, KIF20B, CENPH, LMNB1, GNG5, MZT2B, EZH2, B2M, ANP32B, NME4, DBI, ANXA5, CLIC1, RANBP1, GPSM2, RAN, CD99, VIM, SYNE2, DUT, TAGLN2, IDH2, TMEM98, NKAIN3, DCXR, HES1, SFPQ, KNSTRN, GSTP1, FBLN1, QKI, PXMP2, SRSF2, TGIF1, WDR34, RNASEH2A, EEF1D, LITAF, RDX, HOPX, C12orf75, ID4, SNRPB, RAB13, HADH, ZFP36L1, RHOA, PON2, CLU, TMSB15A, COX8A, GPX4, GINS2, TMEM106C, EZR, SCRG1, SPARC, LIMA1, CARHSP1, UCP2, H2AFY, DNAJC9, AAMDC, SKA2, HNRNPA3, ECI2, PSME2, EMP3, SOX9, COMMD4, SPTSSA, RHOC, SEPT9, PSAT1, ORC6, LSM4, CNN3, CAMTA1, SYPL1, NUDT1, PFN1, DDAH1, STK17A, DECR1, CBX5, ALDH7A1, HNRNPM, VRK1, ITGB3BP, ACAA2, CKAP5, TMEM237, PMF1, HMG20B, ASRGL1, RNASEH2B, MDK, SH3BGRL, NENF, CYBA, ANXA6, PNRC2, MZT1, NFIA, EMC9, NASP, RNASEH2C, ACTL6A, SRI, SLC25A5, NUDT5, RHNO1, GGH, MSI1, PFN2, SEPT11, HDGF, PPP1CA, UQCC2, ACADM, HNRNPD, PHLDA1, PSME1, FUS, CALD1, GSTO1, HADHB, PEA15, MARCKS, SAE1, TPM4, GPM6B, GPC4, MYL6, BLOC1S1, LHX2, LRRC3B, CDK4, EXOSC8, GBAS, CD63, PPDPF, SNX5, GOLIM4, SERF2, NAA38, MPPED2, NFIC, DNMT1, ELAVL1, PAM, CXXC5, TIMM10, NT5C, PGM1, H3F3A, GLUL, HES6, DHRS7, RALY, SNRPD1, PAICS, CCDC14, ASPH, FUZ, HP1BP3, TMSB4X, CYR61, TPGS2, PIN1, RFC2, ID3, PDLIM7, BCAN, IFI27L2, PDLIM4, RPS27L, NDUFA11, VEZF1, FKBP3, HIBCH, GAPDH, JADE1, ANAPC11, BBX, ROBO1, MID1, RPS6, SMARCA1, UBE2E3, MTHFD1, TUBG1, HIGD1A, ATRAID, HSD17B10, TRIM24, LSM14A, KCNG1, FAM96A, CXCR4, PDIA6, POU3F2, HINT2, NPC2, GMPS, CCT5, SHMT2, PFKL, SLC25A6, SEC11A, JAM2, CFL2, PDIA4, APOA1BP, HYI, PRDX6, FUBP1, MAT2A, TTYH1, BAZ1A, PGP, SUZ12, MAZ, EIF4EBP2, CORO1C, CHCHD5, RFC4, PNKD, ITGAE, UQCC3, C1orf61, FERMT2, TMTC4, PTGES3, POLR2E, ETV1, POLR2L, FIBP, PLEKHO1, AHCY, MRPL11, TSPAN6, MYEF2, CALM3, CCBL2, PPIA, PEX2, LRRC58, TXNRD1, HLA-B, IMMP1L, MYH10, TFDP1, CTNNBIP1, HIST1H1C, HSP90B1, UQCRC1, TSEN34, NAA10, CD151, LSM3, TULP3, LSM2, CNTFR, HNRNPUL1, TMBIM4, HLA-C, COPRS, NELFE, NDUFAF3, FUT8, MPST, PRDX3, BAALC, CCNG1, SRSF6, LMAN2, ENSA, MKKS, PRADC1, SUGP2, SCRN1, FZD3, RANGRF, PPIF, FAM92A1, ADH5, RPN2, CNP, SLC35F1, PPP4C, EHBP1, HNRNPAB, MACROD1, ACYP1, SMS, ATP1B3, ZNF738, COX17, MAPK1IP1L, H2AFY2, LRRFIP1, TMEM107, MINOS1, BCKDK, TUBB, MRPS16, MRPL23, ILK, MED30, SSNA1, SNX17, PTCD3, CTBP2, PSMD14, UBE2L3, RRP7A, DPM3, RPL39L, RABL6, MSI2, DGCR6L, CALR, RFXANK, GINM1, SAT1, TMA7, WDR1, SCCPDH, PA2G4, ANAPC15, STX10, C17orf89, GFAP, CHD4, MPDU1, AK2, GPAA1, UBXN4, CYB5R3, HACD3, NDUFS6, TRAM1, CCDC88A, IPO9, ACAT2, PPM1G, CTCF, TFDP2, MYL12A, NUTF2, NELFCD, HOOK3, DARS, CTDSPL2, FDX1, PCBD1, MRPS6, SPAG9, NDRG2, CENPV, GNAI2, CSTB, ARL4A, VRK3, TMEM132B, TIA1, SLC35B2, FN3KRP, CDK2AP2, SNRNP25, DNPH1, NSRP1, HRSP12, AC004556.1, UBE2E1, SLC16A1, SLF1, SUMO3, ARHGAP33, SRGAP2B, RP3-525N10.2, IFT81, GOLM1, C7orf55, ELP6, EXTL2, COMMD10, ID1, CISD2, XRCC6, ISYNA1, PDCL3, SRR, RECQL, CASP3, LPCAT1, TRIM36, STRA13, SESN3, CCND2, PSMA4, DHX15, RNF168, MIF4GD, RIT1, DNAJB1, DHX9, CNPY3, GPT2, TMEM141, REEP3, CST3, ABHD12, CTPS1, SLC39A1, APOO, KCNQ2, PIGX, SLC25A1, PEX10, CUL1, EXOSC3, TAF9B, IQCB1, JPX, NGLY1, PLOD2, CENPT, SRPK1, TUBA1C, TIMP2, PEX19, KLHDC8A, EFTUD2, SGCE, PHYH, TSC22D4, NRCAM, SNAPC1, TOR3A, SPATA33, TMEM38B, and HES4.

In some embodiments, the organoid has been cultured for at least 6 months. Such organoid may comprise one or more of: astroglia, callosal projection neurons, cycling progenitors, immature callosal projection neurons, immature interneurons, immature projection neurons, intermediate progenitor cells, outer radial glia, radial glia, ventral precursors, outer radial glia/astroglia, and cycling interneuron precursors. In some examples, the organoid may comprise one or more of: about 6%-16% astroglia, about 7%-22% callosal projection neurons, about 5%-8% cycling progenitors, about 10%-31% immature interneurons, about 2%-10% immature projection neurons, about 1%-7% intermediate progenitor cells, about 22%-39% radial glia, or about 4%-8% ventral precursors. In some cases, organoid may comprise substantially no corticofugal projection neurons or immature corticofugal projection neurons.

In some embodiments, an immature projection neuron in an organoid cultured for at least 6 months or more is characterized as an organoid cell that overexpresses, as compared to the rest of the organoid cells, at least 5, 10, 20, 30, 40, 50, 75, 100, 125, or all 136 of the following genes: ARF4, DDIT4, SEC61G, EIF1, HERPUD1, PGK1, BNIP3, MORF4L2, ALDOA, IGFBP2, ILF3-AS1, ALKBH5, FAM162A, NPM1, ARF1, SERP1, EGLN3, DDX18, H1F0, ENO1, HILPDA, TMED9, KDELR2, P4HB, HSPA5, SLC3A2, KCNQ1OT1, LDHA, SRP54, TMED2, MYDGF, RPS5, ZFAS1, VIMP, CA9, PDK1, P4HA1, ADM, NRN1, SLC16A3, MIF, RNMT, DNAJB9, SRPRB, INSIG2, HSPA9, NANS, PGAM1, DCAF13, GNL3, GORASP2, BNIP3L, EPB41L4A-AS1, ENO2, ATF4, EIF2S2, TXNIP, XBP1, ZCCHC7, UFM1, WDR45B, RSL1D1, COPB2, ANKRD37, SEC13, ST13, TRIB3, CCDC107, WSB1, PRDX4, BOD1, BET1, EIF2A, DNAJC3, TMEM263, RPF2, RP11-798M19.6, SSR3, TAF1D, SUCO, COPB1, SLC39A7, SEC61A1, TPI1, SURF4, MPHOSPH10, HM13, SEC31A, GOLGA3, IGFBP5, PFKFB3, DNAJB11, GPI, MIR210HG, UAP1, SIAH2, FUT11, EPRS, GOLGA4, MTHFD2, DNAJB2, TMF1, SARS, MXI1, GARS, COPG1, NARF, TNIP1, PPIL3, TATDN1, CCDC47, RPA2, WDR54, EGLN1, PGM3, KIAA0907, ALDOC, SHMT2, AARS, MLEC, SND1, KDM3A, PRPF6, LONP1, EBLN3, EIF4EBP1, EIF2B1, RSBN1, VEGFA, SERPINH1, TET1, FAM210A, ELP2, IARS, ASNS, and RGS16.

In some embodiments, an immature callosal projection neuron in an organoid cultured for at least 6 months or more is characterized as an organoid cell that overexpresses, as compared to the rest of the organoid cells, at least about the first 5, 10, 20, 30, 40, 50, 75, 100, 125, 150, 200, 250, 300, 350, 400, 450, 500, or all 547 of the following genes: PALMD, NEUROD2, BHLHE22, CLMP, CSRP2, SLA, ELAVL2, NEUROD6, CADM2, SEZ6L2, SNX7, CXADR, SNCA, RBFOX2, PPP2R2B, NSG1, CD24, EIF1B, MIAT, GRIA2, RAB3A, ATP6V1G2, ZBTB18, STMN2, SOX11, TSC22D1, NREP, CCNI, GNG3, CPE, MEIS2, SRM, BEX1, THRA, CRMP1, APP, BASP1, RTN1, TMSB10, HN1, PTMA, EIF4A2, SSTR2, ZNF704, BEX2, ATAT1, POU3F3, APLP1, POU3F2, SEMA3C, DUSP1, PLXNA2, ZNF462, VAMP2, SVBP, TTC3, TERF2IP, PODXL2, PHLDA1, LMO3, CAMKV, LMO4, SHTN1, GAP43, MN1, ENC1, FOXG1, TBR1, KLC1, AP3S1, FRMD4B, FAM49B, NRP1, SNAP25, LRRC7, TBPL1, ETFB, CNOT2, TXNIP, EPHA4, CDC42EP3, NELL2, RPAIN, VCAN, HSP90AB1, CNR1, PBX1, CAMK4, AUTS2, IP6K2, IFRD1, TTC28, DOK6, PPP1R14C, SMARCD3, ZC2HC1A, DDX24, CCDC28B, SMIM15, GNAI1, MARCH6, CDK5R1, FAM126A, UBE2D1, HPCA, GABPB1-AS1, CCNG2, CELF2, TM2D3, VDAC3, MAP1LC3A, ENO2, AP1S1, SPTAN1, COX7A2L, PLPPR5, HS3ST1, LINC01102, GNAL, NR2F1, MAPT, PCSK1N, TTC9B, TSPAN5, TNRC6B, CAMLG, NDUFAF2, ITFG1, ARID5B, NUP93, MLLT3, APLP2, TCEAL7, CRYZL1, DAAM1, FAM215B, BAIAP2-AS1, HMP19, YWHAG, FAM13A, MKRN1, NPB, ZNF608, KIF5C, PFKM, RASGRP1, POLR1D, SARS, SCG3, FUT9, BEX4, Clorf216, NRXN1, CMAS, MMADHC, AKAP9, AKR1A1, RRAGA, RPL7L1, TRIM2, NHSL1, UCHL1, NME1, WHSC1L1, GRB2, HSPA8, DEAF1, PTCHD2, ZNF292, TMEM108, IGSF8, RNF24, YWHAH, MAP4, CHD3, EEF1B2, SRGAP1, STMN4, KCTD6, TMEM59L, SLF1, ANP32A, ATP5G1, PID1, SMIM8, FAM57B, SMARCA2, MEX3B, LRRTM2, NTM, BLCAP, CCDC112, DACT1, NUDT3, DDX1, PHF20, RP11-192H23.6, ST3GAL6, WDR47, GPR162, ELAVL3, GNAO1, EPB41L4A-AS1, ARMCX3, MRPL32, GNG2, CCNB1IP1, SPATS2, PWAR6, CEP170, ZEB2, NFASC, GNL3, C1orf52, TRAP1, ZHX1, TIPRL, PHF20L1, CAMK2B, SSX2IP, TULP4, LHX2, IDS, TMEM167B, CLASP2, TBCC, EPHB 1, LDOC1, CELSR2, C5orf24, APBB1, STARD4-AS 1, FAM107B, HK1, GPM6A, EML1, PLEKHA1, OCIAD2, FAM171B, PLPPR2, GALNT11, ANAPC5, CHGB, KNOP1, MPHOSPH8, SPINT2, ZNF148, SERGEF, TSPYL1, AMER2, HSF2, GRIA3, LY6H, MCTS1, DCTPP1, IRF2BPL, IFT20, C14orf132, NT5C3A, ORC4, PGAP1, LEO1, PEBP1, AC004158.3, CHCHD6, CCDC115, RP11-83A24.2, PTPN4, NEO1, APBA2, FSD1, KRR1, ACYP1, ZNF131, EBPL, CMSS1, CNOT4, CD200, PJA1, NIPA2, PRPSAP2, HARS, GPR85, SMAD2, SLC35E3, MAGEH1, FBXL15, PLXNA4, SBK1, CECR5, FARSB, BTBD10, MRPL44, ANKRD46, STXBP1, TACC2, RIC3, C3orf14, ARMCX1, TMEM35, RUFY2, SRSF8, POLR2B, TMED3, AMN1, KBTBD6, FKBP4, TTLL7, FMNL2, TBC1D14, CCDC136, SHOC2, ATL1, ZNF821, RAP1GDS1, ZNF91, BLOC1S6, RSBN1L, TRMT10C, LARP1, COPS3, JPH4, ASNS, CLIP1, PKIA, CES2, F2R, RAC3, SH3RF3, SBNO1, RNF165, ATP6V0A1, PRR7, ACTR1B, CEP57, ZPR1, RAMP2, ATXN7L3B, ZNF397, KIF3A, KIFAP3, SLC4A7, RIMKLB, MYT1L, NIPSNAP1, NDUFAF4, PPP3CB, FKBP1B, LMO1, NFKBIL1, SF3A3, HSDL1, NPM3, LETMD1, RIF1, NAA15, TAF1D, RP11-436D23.1, HDAC5, SRD5A1, PARP2, MRPL48, IGSF3, HINT3, MPZL1, EFNB2, YPEL1, RAP2A, ILF3-AS1, HMGXB4, DERL1, ARRB2, EPM2AIP1, TPT1-AS1, PAK1IP1, PLEKHA5, CDKN1B, CNKSR2, RPS6KA5, PTPRG, WDR33, GOPC, UBQLN2, GTF2B, ASGR1, FNBP1, LRIF1, ZC3H6, WDR82, ZNF766, RNF14, AAK1, ZFAND1, CELF3, XBP1, SERP2, ZNF770, KDM6B, THAP9-AS1, EXOC4, VPS37A, ING4, LINC00667, EIF4EBP1, COIL, SIAH2, BZW2, GARS, KMT2A, SLC35E2B, SH3BP5, CHST12, EIF3J-AS1, C2orf69, R3HDM2, NSMCE3, DIXDC1, EEF1A2, SCAMP1, SORBS1, UXS1, MCMBP, SNHG8, CHMP7, FRMD4A, VPS53, CAMK1D, RP11-1094M14.11, SLC8A1, ZNF622, CUL1, ELP2, NUDT11, MBTPS1, RFPL1S, C12orf65, FAM131A, ZNF7, PPID, ZC3H11A, NOB1, PUS7L, KAT8, CLK1, PPP1R10, MRPS2, FBXO22, PAK1, SLC35A1, ACOT7, MYCBP2, NOL11, THUMPD1, ITSN1, TMF1, FBXO44, PEX13, CBFA2T2, FAM217B, CLK3, ERAL1, RABIF, TUBGCP4, ATCAY, B4GALT3, GDAP1L1, RSBN1, KBTBD7, ARMC8, SYP, FSD1L, GADD45G, SNAP47, KLHL23, CSAD, TTF1, GNB5, CELF5, PHF1, BORCS8, SNHG15, ZMYND8, CDKN2D, GDAP1, PPP2R5B, HOOK2, ZFP90, MPHOSPH10, TCAF1, ZNF512, LIN7B, NOC2L, PGM2L1, PCGF2, OGFOD1, IGDCC3, NECAP1, G3BP2, SFSWAP, ACTL6B, FAM49A, FAM126B, NUDCD3, B4GALNT1, EXOSC5, SEZ6L, BBC3, SDAD1, ERICH1, REEP1, CASC3, MTPAP, C9orf72, YDJC, PURB, THAP3, RUNDC3A, BEND5, ARIH1, HPRT1, RP11-352M15.2, RPAP2, RIOK1, DPH7, WDR74, KLHL28, WASF1, ATP1A3, LARP6, DYRK2, INAFM1, CELF4, CCP110, ZNF652, NRBF2, NPRL2, NAT9, TMEM57, NETO2, GSK3B, GFOD2, FNIP2, PIK3R1, KCNQ1OT1, ARPP21, PLK2, and INA.

In some embodiments, a callosal projection neuron in an organoid cultured for at least 6 months or more is characterized as an organoid cell that overexpresses, as compared to the rest of the organoid cells, at least about the first 5, 10, 20, 30, 40, 50, 75, 100, 125, 150, 200, 250, 300, 350, 400, 450, 500, 600, 700, 800, 900, or all 914 of the following genes: FGF12, MEF2C, LINC00643, TSPAN13, SYT4, GRIN2B, ARPP21, SYBU, MPPED1, PAK7, SH3GL3, NEFM, RBFOX1, JAKMIP1, SEMA7A, CAMKV, INA, TTC9B, PIK3R1, LINGO1, NELL2, R3HDM1, CCBE1, CAMK2B, HPCA, DUSP23, CELF4, MMD, FAM49A, CXADR, NSG1, GNAI1, HMP19, SYT1, SPINT2, SHTN1, SLA, SNCA, ENC1, STMN2, DACT1, RAB3A, CDKN2D, STMN4, BHLHE22, LY6H, SEZ6L2, LMO4, ZBTB18, RAC3, ATP6V1G2, NEUROD2, CD24, TSC22D1, YWHAH, DOK5, UCHL1, GAP43, MAP1B, CRMP1, STMN1, TUBB2A, BEX2, VAMP2, BASP1, GNG3, RTN1, MLLT11, PCSK1N, HN1, SCN3B, PTPN2, CADM2, INAFM1, BEX5, PGM2L1, ATP2B1, FABP7, SULT4A1, CADM3, SSTR2, BEX1, GPR85, SYT13, CDC42EP3, SATB2, ADCY1, RASL10A, MIAT, PCLO, TAGLN3, MYT1L, DEAF1, ATP6V0B, AKAP7, FKBP1B, YWHAG, GPM6A, PPP1R14C, APLP1, DLG2, CALM1, NEUROD6, RGS17, DAB1, SCG3, GABBR2, CDC42, TUBA1A, HOMER1, PLPPR5, BEX4, SERP2, TMOD1, DSTN, C1orf216, PAFAH1B3, OCIAD2, SYT5, ATP1B1, HBQ1, MAP1LC3A, PPP3CB, FAM49B, PLK2, KLC1, GNAZ, FJX1, EIF4A2, TBCB, GABRB3, TPM3, RBFOX2, DYNC1I1, DPF1, PRR7, RBFOX3, HS3ST1, WASF1, ACOT7, SNAP25, ATL1, CDK5R1, CHGA, CELF5, NREP, HSP90AB1, RUNDC3A, C1QTNF4, TUBB, RNF165, PEBP1, VSTM2B, AASDHPPT, SNX7, CLMP, ARPC2, GPRIN1, ETFB, YWHAZ, FAM57B, CSRP2, RP11-356J5.12, F12, DNAJB6, GDAP1L1, CLTB, TMEM59L, TUBB2B, DOK4, ATP1A3, SCN2A, CORO1A, LY6E, KIAA1107, PFDN2, PPP2R2B, GNAL, CELF3, SLC8A1, TMEM14A, SLC38A1, LRRC7, PPFIA2, LIN7B, PRKCZ, REEP1, TMEFF2, PPP3CA, PCMT1, BZW2, PODXL2, SH3BP5, ATCAY, AP1S1, L1CAM, PHACTR3, HS6ST3, STX1A, CAMK4, HIVEP2, HSPA8, ASPHD1, EPHA4, YWHAB, ARPC5, CSRNP3, ELAVL3, RNF187, EXOC4, EFNA3, TAF9, MIR124-2HG, PPP2R5B, ATAT1, DLL3, YPEL3, LDLRAD4, CALM3, PLPPR2, SRM, MAPT, TXNIP, GRB2, NPB, COTL1, BCL7A, RAB33A, NUDT3, NRXN1, DRAP1, MYCN, GFOD1, THY1, NTRK3, CHGB, RFPL1S, ACTL6B, TCEAL2, ADD2, NDUFA5, PTPRO, ANKRD46, TM2D3, C6orf1, ANKRD12, CSNK1A1, AFF3, RAMP2, ATP5G1, CHD5, ARG2, TMEM160, DAAM1, NAP1L3, NUDT11, SMAP2, POU3F1, STXBP1, RNF182, DISP2, KIF3C, PRKAR2B, LINC00599, CDK5, SSX2IP, STOML1, OLFM1, BLCAP, RFK, PNMA1, CMAS, NDRG1, MAPK8, RAP1GDS1, TSPYL1, HCFC1R1, TULP4, UBE2V2, PSD, DDX24, SLC25A4, SERINC1, NECAP1, PLPPR1, SYP, CTXN1, TNNT1, COMTD1, FAXC, ILF3-AS1, GTF3A, FBXL15, MARCH4, AUTS2, MPP6, FLRT2, NME1, CYCS, ENO2, PTPRD, NDUFAF2, PRKACB, CA11, MAPRE3, EML1, RUNX1T1, VPS29, CD200, NAPB, NUDCD3, ANK3, ACTR3B, ST6GAL2, NMNAT2, CHCHD6, AP3S1, ARID4A, TCTEX1D2, ZBTB38, ST3GAL6, CCDC112, SRD5A1, CDKL5, CELF2, GRAMD1A, SOBP, GFOD2, HSDL1, KIF3A, NUDT14, TMOD2, AGTPBP1, DIRAS1, TTC9, GABRB2, H1FX, TUBB4A, KIF5A, ATOX1, TMEM35, CACNA2D1, C10orf35, TMEM150C, THRA, FGF13, BID, CDKN2AIPNL, APBB1, WAC-AS1, C5orf24, C2orf69, RIC3, C9orf16, SBK1, FNDC4, SRRM4, TTLL7, SLBP, MKRN1, YDJC, IDS, ZC3H15, AKT3, KLHL8, MORF4L2, NEO1, SNAP91, ZEB2, TCEB1, DPYSL5, FSD1L, EIF4EBP1, NDN, STRBP, PARP6, RASSF2, KIF5C, FSD1, C12orf76, MPC2, PARD6A, RGS7, FAM134A, ST3GAL1, ATP6V1B2, NBEA, RPS6KL1, GNB5, TMEM57, KCTD13, NDEL1, PPFIA3, PAK1, DEF8, GNAO1, ASXL3, CAMLG, RELL2, MEAF6, CAMK1, KIF3B, HK1, COX7A2L, HIVEP3, SPTBN1, CACNB3, JPH4, ELOVL4, CCDC184, TBC1D14, MEX3B, CDH11, TIPRL, KIDINS220, BAIAP2-AS1, BTBD10, DTX3, TMEM151B, TMEM108, TCEAL7, DCAF6, MYCBP2, KIAA0895L, SLC12A5, GABPB1-AS1, ANKS1B, CPE, PEX13, FNIP2, FAM126B, PTBP2, NOL4, PLXNA4, HDAC5, DLGAP1, POP7, RNF11, PPP3R1, CELF1, LHX2, BORCS8, KBTBD6, PLPPR4, SCAMP1, KLC2, KIFC2, AMD1, MAST1, DCTN3, KIFAP3, SEC11C, ZNF821, PPID, FARSB, RP11-127B20.2, GOT2, NCOA1, NTM, FAM126A, ARL10, HSD11B1L, RAB2A, CNR1, GRIA1, ANK2, RABEP1, GNL3, SV2A, MAP4, PPP2R1A, MRPL18, VTI1B, RUFY3, SCAMP5, GNB1, RP11-352M15.2, ACYP1, PHAX, YPEL1, WDR47, ATP13A2, ROGDI, GNG2, PHACTR1, CCDC90B, HINT3, C17orf58, USP11, RAPGEF2, BBC3, IGSF3, SEPT6, AFAP1, PITHD1, GIT1, PRDM2, FRMD4B, SMIM8, FAM117B, CRK, FAM188A, SLC35E3, TSPAN14, ODF2L, SLC44A5, PKIA, FAM155A, DDX25, RIMKLB, GPR162, RP11-382A20.3, SBNO1, ATP6V0D1, MAP6, CLASP2, EPHA5, MPZL1, ARHGEF7, KLHL23, PDIK1L, PCDH7, ZMAT2, MAPRE2, RNF219, C16orf45, TNRC6B, ARF3, FAT3, CMIP, SPOCK1, AK1, RRAGA, ZNF302, NRXN2, CDK19, CAMKK2, KIF2A, ATXN7L3B, ITFG1, LINC00657, DYRK2, C9orf78, ARHGAP33, PBX1, PAIP1, AMN1, TRIM3, RUSC1, CCSAP, MICAL3, PJA1, TMEM178B, SSBP4, PRKAR1B, ATXN10, MSRA, SHOC2, SPIN1, PSMG4, PTP4A1, ZBTB44, ZNF148, ZWILCH, DTNBP1, PNMA2, OPTN, DTD1, FRMD3, B4GALT5, MAP7D1, CEP126, DUSP8, MYO5A, ZNF622, CACNG8, NAP1L5, SPTAN1, TSPO, ST8SIA2, MAGEF1, TRAPPC4, TBRG1, SESTD1, UBQLN1, FAM131A, TCAF1, SLC16A14, LINC00632, RABEPK, UBL4A, ARMC1, SERINC3, ITSN1, FAM89B, ZC3H6, PLPPR3, MRPL44, ATP9A, SORBS2, VPS4A, CDC37L1, PAK1IP1, LDOC1, DYNC1LI1, HOOK2, RAB14, RNF113A, ASNS, SNHG15, ZNF793, TNFRSF21, PAFAH1B2, TOMM70A, RIMKLA, KALRN, MCUR1, ENDOG, C1orf52, RNF146, RP11-83A24.2, TMOD3, TCP1, C12orf73, PPP1R9A, CTTNBP2NL, ZNF74, DYNLRB1, IRF2BPL, GALNT11, ALAS1, CCP110, CNIH2, SMARCD3, LINC00667, LSM10, CCDC136, SS18L2, RNF145, TSPYL4, NT5C3B, SRPK2, CACYBP, B4GALNT1, KATNB1, BRSK1, RABL2B, AGAP3, FAM217B, MIR181A1HG, BOP1, IGSF8, FARP1, AHSA1, SH2B2, PDZD4, FKBP4, PAFAH1B1, HARS, PCGF3, PRR3, NETO2, LONRF2, HEBP2, DIXDC1, ENTPD6, SCAI, RALA, PRKAR1A, AAK1, RNASEH1, PIP4K2B, TRAPPC6B, ZNF281, ATP2C1, TRIM2, CLIP1, KIAA1549, SEPT3, PSD3, ZNF566, GPR161, RP11-192H23.6, DUSP6, EXOSC6, MAPKAPK5-AS1, LTBP4, NIPSNAP3A, COPS7B, GOPC, COMMD9, STMN3, ELOVL6, STOX2, G3BP2, ACVR1B, ADGRL1, SRCIN1, BDP1, GRIA3, PKN1, PARP2, TIMM17B, SEC14L1, RBM15B, ERC1, CSNK1E, EPM2AIP1, MED13L, TMEM167B, PIANP, ATP1A1, TRUB1, MORN4, HMOX2, KNOP1, THAP9-AS1, PCSK7, KIAA2022, MAP9, ZYG11B, SGSM3, IFT20, MED19, NOLC1, AMER2, FOXJ3, AC004158.3, LETM1, MAP1A, GPRASP2, ATP6V1A, KMT2A, MAX, DPYSL3, EGLN1, GRIN2A, GNAQ, BAG4, BOLA3-AS1, LIN7C, PTDSS1, MAP1S, EPN1, TAF6, UBALD1, SLC25A17, ATP6V0A1, WDR82, MRFAP1L1, UBE2O, SLC25A36, XXYLT1, SH3RF3, DNAJC12, RPAP2, NFASC, CHORDC1, UGCG, ZNF652, TSSC1, UCHL5, PPP1R2, IFNGR2, UCHL3, SFXN3, BLOC1S6, CHRNB1, FEM1B, SPPL3, DNM1L, CAMK2G, TRAPPC2, ZMYND8, FAM228B, TMEM192, SNAP47, INPP4A, PTPN1, CHN1, RHOB, FAM177A1, UBQLN2, IRGQ, NOVA2, LRRC49, EIF2AK4, BRD2, RPUSD3, C15orf57, NR2C2AP, CCDC107, HERC1, CAMK1D, EXOSC5, MTMR4, CAMSAP1, UBE2Z, RALGDS, ZFAND2A, CCDC186, FBXO45, GPATCH2L, DCAF10, NAV3, Clorf21, ARRB2, TSPAN5, DDX51, TSPAN7, OGFOD1, ZMYND11, DUSP12, FEM1A, HSPH1, CENPT, SEH1L, NAA15, LRP12, GAREM1, LRRC40, ZC3H8, ZNRF1, ZNF445, ISYNA1, MTMR9, RALGAPA1, AKAP11, KIAA0930, ZBTB37, CAMSAP2, C11orf95, CSRNP2, SLC35B4, TRAP1, RAB6A, PPP1R18, JARID2, C16orf72, ANKRD13D, GGT7, HCG18, GMEB1, R3HCC1, SLC22A17, GABPB1, PDHA1, GFPT1, CC2D1A, LARP1, DCTN5, BMPR2, DCTN1, AKIP1, CCZ1, DCAF7, ZNF32, RP11-660L16.2, LRP3, MLXIP, ATP6V1H, NUTM2B-AS1, MSL1, ATAD1, CAND1, CAP2, ABL2, VPS53, MTURN, CLIP3, TRIO, R3HDM2, ZFAND2B, SECISBP2L, FAM219B, ASGR1, SMARCA2, PPIL2, DERL1, ABR, ADGRB3, NCOA6, JOSD1, ABHD6, ARHGAP35, PRCC, EIF2B2, MYO9A, CUL2, FAM98A, USP7, BAIAP2, HECTD4, ANKRD36C, WDR20, MLX, SAMD14, SIGMAR1, FHOD3, NAV1, ISG20L2, POGK, PDXDC1, SBF2, YY1, ARHGEF12, ZNF639, SHISA5, ARHGEF9, ATMIN, GATAD2B, EXOSC10, ZNF512, and PANK3.

In some embodiments, an intermediate progenitor cell in an organoid cultured for about 6 months or more is characterized as an organoid cell that overexpresses, as compared to the rest of the organoid cells, at least 5, 10, 20, 30, 40, 50, 75, 100, 125, 150, 200, or all 235 of the following genes: EOMES, CPE, TMEM158, CLMP, MLLT3, RASGRP1, SMARCD3, SEZ6L, SERPINF1, UNC5D, MARCKSL1, CXCL12, PPP2R2B, CNR1, GNG3, CYB5A, CNTNAP2, SOX4, TBR1, CDC42EP3, MEIS2, CORO1C, GPM6A, SSTR2, LYPD1, GAP43, DOK6, TFAP2C, CSRP2, MLLT11, UBE2E3, EEF2, IER2, RPAIN, TMEM108, ASCL1, ZFHX4, MAPRE1, EPHA3, CALD1, MN1, POU3F3, ZBTB20, FMNL2, MIR99AHG, EBPL, PGRMC2, KIAA1715, POU3F2, MYO6, RP11-436D23.1, NR2F1, IGSF10, RP11-553L6.5, MAGED2, FRMD4A, SCARB2, PTPRS, TMSB10, MTCL1, ATP1B3, DAAM1, SYNE2, UBE2D1, FIGN, LSAMP, PBX1, CMC1, DDAH2, RBPJ, LUC7L3, LRP8, SEZ6, SRGAP3, EPHB1, GPRCSB, CIRBP, STMN4, DLL3, C12orf49, CCNG2, ZHX1, FUT9, BLCAP, PHYHIPL, RAB3A, MAP2, BTG2, GULP1, BBX, TERF2IP, OSBPL6, GADD45G, MEX3B, TRIM2, FAM126A, BAZ2B, GTF2I, SETD7, INSM1, EML1, ABRACL, ZC2HC1A, ARIDSB, BICD1, GRIA3, ATP6V1G2, C1orf54, TFDP2, MPPED2, PRMT1, RPN2, NUP93, EMX2, VCAN, SRGAP1, WIPF3, HEBP2, IGFBP2, FZD3, TIMP3, MDK, PCBP4, NFKBIA, MLLT4, SCRN1, MLLT4-AS1, ELAVL2, FGF13, DLEU1, SPIRE1, KNOP1, C14orf132, MIDN, ATAT1, LPCAT1, NFIX, AIP, BEX1, TCAF1, TANK, KDM5B, CYTH1, MDFI, ITGB1, HDAC9, DTD1, APLP1, EVL, GSTA4, HDAC5, TP53RK, SEMA6A, MBTPS1, BMPR1A, PJA1, ARL4C, ZMIZ1, LDOC1, LHX2, PCMTD2, SPATS2, CDK5, CPNE1, LTA4H, ELMO1, NARF, INTS6, TNRC6B, IP6K2, IVNS1ABP, ZBTB18, ZKSCAN1, RPA1, RP5-1085F17.3, GPC2, RP11-76114.1, NTM, POU2F1, TBPL1, HERC2, FNBP1, CALCOCO1, PLPPR1, BEX2, LINC01102, SOBP, CXXC5, NIPSNAP1, HPCAL1, SENP6, RBFOX2, KDM6B, STARD4-AS1, QSER1, NF2, CAMK2G, Cl5orf61, ING4, AC013461.1, BCAR1, MEX3A, APBA2, CBFA2T2, IFI44, SLC39A10, HSDL1, LIN7B, GRAMD1A, SMIM8, USP3, PLEKHA1, TBCC, R3HDM2, ATP6V0A1, AUTS2, RAB8B, IRF2BPL, GDAP1L1, TMTC2, FOXN2, SYP, USP46, FAM217B, KLF3, CPT1C, AC004158.3, HSD17B11, ADNP, CCSAP, PCDHB2, UBALD1, SOGA1, SBK1, and FAM60A.

In some embodiments, an immature interneuron in an organoid cultured for at least 6 months or more is characterized as an organoid cell that overexpresses, as compared to the rest of the organoid cells, at least 5, 10, 20, 30, 40, 50, 75, 100, 125, 150, or all 155 of the following genes: DLX6-AS1, DLX5, SP9, PLS3, ARL4D, GAD2, TAC3, DLX1, DLX2, MEST, ARX, RASD1, ELAVL4, RND3, TMEM123, CCDC109B, DCX, PFN2, TCF4, SOX4, TMEM161B-AS1, ENAH, TMSB10, HMGN2, ACTG1, HNRNPK, DDX5, TUBA1A, ACTB, H3F3A, SH3BGRL3, RPS11, DCLK2, DPYSL3, DYNC1I2, SLC25A6, AES, ST18, HNRNPA1, DBN1, SMARCB1, HDAC2, CADM1, OLA1, PAIP2, PFDN4, DLX6, ARL4C, FXYD6, TRIM13, CCDC88A, TMSB15A, UBE2I, MSI2, NME6, H2AFY2, MAP2, CITED2, RBBP4, GAD1, KLHDC8A, SMARCA4, ROBO2, CRIP2, NFIA, PRKX, BCL11A, CHD7, SUB1, HTATSF1, TSC22D2, FSCN1, DST, SMARCE1, PAK2, CENPV, PTS, TOX3, PNRC1, BCL11B, MGEA5, NAP1L4, DLGAP4, SRSF6, CBX1, KCNQ2, ARL6IP6, FAM89B, RPA1, CHD4, RNASEH2B, POU2F1, CORO1C, SMARCD1, KLF7, MLLT4, KAT6B, PHF14, ATP2B4, LRRN3, FOXO3, ANAPC15, TDG, SERINC5, CREB1, PAK3, GPC2, PEG10, FAM210B, CERS6, SPATS2, XRN2, ASAP1, INSM1, RBP1, TIA1, LRRC40, SECISBP2, ACIN1, GSE1, CHD3, SP8, BAZ1A, FOXN3, CELF1, CASC15, MEX3A, SMARCC1, CDCA7, RAB8B, SP3, RARS2, MAGI1, LIMD2, VEZF1, GADD45G, CCDC112, DPYSL4, TCF12, PLK2, ERV3-1, HMGB3, USP3, MED17, RBM4B, CMIP, ZNF3, RAB3IP, PHACTR4, SMOC1, TIAM2, FAM60A, SEZ6, GLCCI1, and LINC01315.

In some embodiments, a ventral precursor in an organoid cultured for at least 6 months or more is characterized as an organoid cell that overexpresses, as compared to the rest of the organoid cells, at least 5, 10, 20, 30, 40, 50, 75, 100, 125, 150, 175, 200, 250, 300, 350, 400, 450, 500, 550, 600, or all 605 of the following genes: DLGAP5, ASPM, UBE2C, CCNB2, TROAP, FAM64A, TTK, NUF2, CDCA3, CENPF, MKI67, GTSE1, CDCA8, KIF23, KIF2C, PTTG1, TPX2, CDKN3, CCNA2, NUSAP1, BIRC5, KIF4A, SGOL2, TOP2A, AURKB, PBK, HJURP, PRC1, TACC3, CASC5, SGOL1, ECT2, CKAP2L, KIF11, NDC80, MXD3, CDK1, ARHGAP11A, DEPDC1B, HMGB2, CRNDE, KIFC1, CKS2, KNSTRN, KPNA2, SPC25, RACGAP1, MIS18BP1, CKAP2, MAD2L1, CDC25B, KIF20B, SMC4, UBE2S, CENPW, CENPN, TUBB6, KIF22, TUBB4B, UBE2T, CDKN2C, CKS1B, H2AFX, ZWINT, HMGB3, MZT1, SMC2, LMNB1, TMPO, TUBA1B, BUB3, H2AFZ, H2AFV, RAD21, ANP32E, HMGN2, LSM5, HMGB1, NUCKS1, HNRNPA2B1, RAN, YBX1, RBMX, DCXR, BUB1B, DTYMK, SPC24, NCAPG, CENPU, RTKN2, EMC9, SFPQ, OIP5, SPAG5, DBF4, KIF15, TYMS, GPSM2, FOXM1, KIAA0101, MELK, MND1, FBXO5, DDX39A, HIST1H4C, EZH2, PSRC1, ILF2, LBR, CENPK, POC1A, RRM2, SKA3, STMN1, TMSB15A, HNRNPM, PARPBP, SRSF3, CENPM, SHCBP1, RAD51AP1, SKA2, HMGN1, KIAA1524, DEK, H3F3A, MARCKS, CCDC34, NCAPH, HNRNPA3, SPDL1, TUBB, CENPH, C21orf58, ESCO2, DLEU2, SKA1, RHNO1, NCAPD2, HNRNPR, VRK1, KMT5A, CMC2, ASRGL1, HES6, PSIP1, ERH, CDCA5, LRR1, GPX4, TRIP13, PLK4, CLIC1, EEF1D, ASCL1, HNRNPH3, LSM4, NUDT1, GGH, CCT5, NCAPG2, DAZAP1, PSMA4, ANP32B, USP1, FBLN1, UCP2, NPY, PIN1, CKB, FANCI, ASF1B, RCC1, CBX1, SAE1, PTMA, CDCA4, CEP57L1, DIAPH3, FUS, RAB13, C12orf75, BARD1, EXOSC8, MIS18A, HNRNPA0, GMNN, PCM1, RFC3, HNRNPDL, FUBP1, TPM4, ANLN, LMNB2, DLX1, SNRPG, ACTB, CENPC, SEPHS1, CDK5RAP2, HNRNPU, CHEK2, ORC6, SEPT10, CEP135, BTG3, SNRPB, PHF19, RBM8A, COQ2, PSME2, RANBP1, ACTL6A, DLL1, ZEB1, CENPJ, GSX2, RAB3IP, TXNDC12, TAC3, DESI2, TRA2B, IKBIP, RNASEH2A, GAD2, PIM1, CBX3, JADE1, FANCD2, DCP2, BANF1, PDGFRA, DLX2, GNG4, SMC3, BRCA2, UHRF1, EIF5A, H2AFY, TK1, CHIC2, RPA3, BCHE, NAP1L1, MAD2L2, HNRNPUL1, ZWILCH, HAUS8, CHTF18, SMC1A, SUGP2, RNASEH2B, WEE1, PFN2, PTGES3, TMEM237, SAC3D1, RDX, SRSF7, SRSF2, PKMYT1, NUP35, PPIA, RPL39L, CDK6, ATAD5, CEP97, USP13, DCLRE1C, TPRKB, SYNE2, LCORL, CBX5, DNAJC9, INSM1, RRM1, LINC01224, ANAPC15, CCDC167, NEDD1, TIMM10, SNRPD1, CORO1C, MAGOHB, TUBG1, HNRNPH1, C4orf46, PHGDH, NCAPD3, RALY, NUP37, MPHOSPH9, TFDP2, PCBP2, RHOBTB3, GAS1, ANAPC11, QSER1, ATAD2, ACYP1, C18orf54, ITGB3BP, G3BP1, NONO, GINS1, WDR34, SEPT11, MPST, CSE1L, CCDC109B, CEP152, HDAC2, MAZ, TMEM106C, TCF12, PRADC1, MAGI1, TEX30, TPR, SYNCRIP, ILF3, PSMC3, GMPS, SRSF1, LSM8, PHIP, WHSC1, SSRP1, LSM14A, FANCG, SIVA1, ODC1, CEP131, ITGAE, XRCC6, IDH2, PRIM1, CKLF, ELAVL1, MED30, EGFR, MCMI, SMS, IFI16, PGP, CTCF, SNRPC, BAZ1A, ITGB1BP1, CHD7, TIMELESS, TLE1, ARHGAP33, CBX2, NT5DC2, BRCA1, BCL7C, ENY2, RFWD3, HAT1, PTBP1, HNRNPAB, SNRNP40, SRSF10, TOX3, POLA2, UPF3B, NASP, NUP107, PMF1, RFC5, CCDC14, HNRNPD, SUV39H2, SET, NUP62, CNTLN, NUP50, MYH10, MYBL2, HAUS6, XRCC5, PFN1, FBL, NSMCE4A, SERINC5, LSM3, CPSF6, SNRPD3, FUZ, DKC1, NELFE, DSN1, KDELR2, SNRPA, HN1L, ALG8, CENPQ, FKBP3, HIRIP3, HAUS1, SMARCC1, CACYBP, FAM60A, CAMTA1, VBP1, XPO1, SRPK1, COMMD4, PSMB3, HMGXB4, CA14, ZNF738, TMX1, TUBA1C, FAM136A, RBBP7, CBFB, PPIH, CBR3, LSM6, NFYB, CTDSPL2, MAT2B, CEP57, TULP3, KPNB1, UQCRC1, LUC7L2, GNB4, KATNA1, GLCCI1, UQCC2, TIA1, FEN1, RAB8A, NFATC3, SLBP, TBCD, MAGOH, ANP32A, PAICS, MTFR1, AAAS, TARDBP, H2AFY2, PLXNC1, CTNNBL1, GEMIN2, C16orf87, CPSF3, DCTPP1, TEAD2, HSD17B10, UFD1L, SRRT, NME4, THRAP3, NUDT5, SP8, IGF2BP3, CEP78, CSTF1, FAM76B, CHCHD3, EHBP1, ING3, PA2G4, PPP1CA, OLA1, POLR2D, TMEM97, CDT1, CHAF1A, ZNF714, ARFGAP3, MRE11A, POLR2E, SKP2, HNRNPL, DHX9, NSRP1, SF1, STAG1, CTNNBIP1, SRGAP2B, TOPORS, CDK2, VEZF1, MFGE8, EIF4EBP1, PIP4K2A, DHFR, NKAIN3, TMEM18, MCM4, FAM104B, CASP6, C19orf48, DCPS, TRIM24, ZBED1, SCAF11, LRRCC1, GMCL1, RCC2, GINS2, HADH, DSEL, SUMO3, THAP9-AS1, PRKDC, ZNF680, RNF168, TOPBP1, TP53I13, PKNOX1, NUDT21, RBM14, ZNF273, CHRAC1, MMS22L, CCP110, RSRC1, SLC36A4, PPP2R3C, PSMB9, NCAPH2, RRP7A, INIP, CRB1, STT3B, CDCA7L, CTPS1, CEP89, ING5, EXOSC9, ALDH9A1, EMP2, TCERG1, NUP54, BAZ1B, PPIL1, DHX15, PDS5B, RBM25, BRD7, LARP1B, ECI1, CERS6, ASAP2, EIF1AY, ANKRD10, DNAJC2, ILKAP, RNASEH2C, NIPA2, CHEK1, SMAD9, CCBL2, TNPO3, TFDP1, USP39, KAT7, PAQR4, NUP88, LYAR, TWSG1, CLIC4, ACTN4, AGO2, PRR34-AS1, DHTKD1, NUP155, SPCS3, ASH2L, SF3B3, CDYL, AHCY, MLH1, DHRSX, CMTM6, SAAL1, U2AF2, UBR7, MCRS1, ATG4D, PHTF2, NUP58, PPM1D, PSMG1, MOB1A, SMC5, CHD1, ZNF92, MEST, MRPL23, SMC6, THOP1, ARL13B, ZFP91, KHSRP, C4orf27, MBD4, and MACROD1.

In some embodiments of the organoids described herein, an astroglia in an organoid cultured for at least 6 months or more is characterized as an organoid cell that overexpresses, as compared to the rest of the organoid cells, at least 5, 10, 20, 30, 40, 50, 75, 100, 125, 150, 175, 200, 250, 300, 350, 400, 450, 500, 550, 600, 650, 700, 750, 800, 850, or all 893 of the following genes: NTRK2, TPPP3, GJA1, S100A10, AGT, PIFO, ANOS1, GRAMD3, IGFBP7, NMB, CRB2, RARRES3, CRISPLD1, BBOX1, OGFRL1, CD44, CTSH, C1orf194, ITGA6, GADD45B, DCLK1, GFAP, ITM2C, CLU, AQP4, FIBIN, PLP1, HES1, IGFBP5, HEPACAM, KCNN3, B3GAT2, PAQR6, HEPN1, FAM107A, RGMA, TSC22D4, PRDX6, CCDC80, CEBPD, APOE, ZFP36, CD99, ADD3, PLTP, LAMP2, BAALC, EMP3, CDO1, ANXA1, CA2, DTNA, FSTL1, PLA2G16, F3, METRN, ZFP36L1, TSPAN3, PSAT1, SCRG1, CD9, CD81, MLC1, DDAH1, B2M, TTYH1, AP1S2, ENHO, GPR137B, TIMP2, GPM6B, PHGDH, ATP1B2, QKI, PMP22, S100B, ID4, NPC2, CRYAB, BCAN, AK1, SPAG16, NDRG2, VIM, PON2, DNER, NLRP1, HLA-C, CNN3, SOX9, SH3BGRL, MT-ND2, GABARAPL2, MT-ND3, MT-ND1, PMP2, PRDX1, EZR, TNC, ITM2B, SEPW1, MT-CO1, PSAP, MTRNR2L1, PEA15, CST3, FOS, MT-ND4, MT-CYB, PTPRZ1, GCSH, DBI, LGALS3, MT1F, ANXA5, SSPN, ERBB2IP, CTNNA2, NEAT1, AC015936.3, MT-ATP6, AHNAK, C5orf49, RHOC, CSPG5, RHOA, SNX3, RAB31, TIMP3, HLA-A, HIGD1A, ALDOC, SOX2, SLC1A3, TPST1, MPC1, ACYP2, VCAM1, LAMP1, CD38, PSRC1, TRIM47, TMEM47, GALNT15, SPTBN1, DKK3, HSPE1, ZFP36L2, PTGDS, JUNB, C1orf122, TMED10, OAT, CHPT1, CETN2, MGST1, ATP1A2, GLIS3, CIB1, FBXO32, CTNND2, S100A16, LYRM5, IQGAP2, RNF19A, PLEKHB1, CNRIP1, ADCYAP1R1, UG0898H09, FEZ1, GDPD2, CSTB, FAM198B, AHCYL1, GLIPR2, DDR1, MT-CO2, PAM, DST, ALDH2, CD59, TAGLN2, SERPINB6, ARHGAP5, MORN2, DNPH1, TM7SF2, LINC00998, KLF6, SOD2, GNPTAB, CD63, APC, GPRC5B, FAM181A, COPRS, ZFYVE21, ADGRG1, ANXA6, NFIA, SEMA5A, TMEM9B, ACO2, MGAT4C, PLPP1, MLF1, DCLK2, SFT2D1, SCD, SPARC, SCD5, FERMT2, WLS, OSGIN2, HADHB, ID3, ALDH7A1, PTTG1IP, EPB41L3, ARRDC4, CBR1, PBXIP1, TIMP1, BLVRB, HSD17B12, DPP7, SDC3, CAMK2G, FHL1, CRYL1, POLE4, LPAR4, RHEB, PHLDA3, BDH2, ELOVL5, LGALS3BP, MTRNR2L12, LAMTOR4, LIFR, PPP2CB, GNAI2, PFKFB3, PDLIM4, SELENBP1, HLA-E, SORL1, PLPP3, FEZ2, SCN1A, CFI, UBE2E1, COMT, LRRC17, ARL6IP5, ADGRV1, PDLIM2, TMEM255A, KIF9, LRRC3B, ATP6V0E1, CTNNA1, ASAH1, CANX, PRCP, RFX4, MTRNR2L10, UBL3, TMBIM6, ZNF385A, NKAIN3, DOCK7, SEPT2, GBAS, DAAM2, GNG12, TNFRSF1A, PTRF, SQSTM1, PPP1R1C, FAM181B, JAM2, SDHC, ACTN1, SLC7A11, MOXD1, SPTSSA, REEP5, ID2, PDCD6, MAPK8IP1, KLHDC9, TMEM132A, TRIM9, DHRS4L2, AIG1, HINT2, EFEMP2, IL33, C1GALT1, PSME1, PSENEN, NPDC1, PPT1, LRRCC1, FKBP2, SYPL1, CASC4, NFE2L2, NAMPT, CHPF, ABCA1, C1orf54, ADK, SCARA3, SCP2, RAB5A, PTPRA, NDFIP1, LINC00844, EDNRB, ASPH, DAD1, FADS2, SPECC1, EFHD1, MAPK1, MAN1C1, RAB7A, CXXC5, PLEC, PTCHD1, FAM213A, ACAA1, PDPN, UBE2H, ST5, YBX3, NADK2, GAS2L1, DECR1, TP53I3, IRS2, NCAN, PLCD3, MID1IP1, PRUNE2, IFI44L, EPDR1, NUDT4, NDP, EMC2, NDUFB5, ACAA2, HACD3, ADAM9, LRIG1, CYR61, VAMP3, LRP1, TMEM163, DAG1, MLLT1, SIRT2, NME3, SDCBP, RNF13, CTSL, DHRS3, SLC25A18, SBDS, PEPD, SESN3, CH17-189H20.1, GTF2F2, PSME2, TNIK, DPY19L1, STON2, SOX21, SEPT8, PLSCR1, TP53TG1, CDC42EP4, MT-ND4L, PRNP, ELN, ACADVL, SLC25A5, SNX5, LTBP3, PCDH9, B4GAT1, DAZAP2, LIX1, NES, SLC9A3R1, LAMB2, TMEM134, CHCHD5, IGDCC4, MYO10, ENKUR, IGFBP4, OBSL1, PHYHIPL, PPM1K, SEC11A, VMA21, ROM1, AR, CRIPT, NPAS3, APC2, GNA13, RAP1A, NAV1, RCN1, LRP10, SPCS1, ITPR2, EFHC1, PKIG, DDX3X, SEC22C, ANXA7, RP11-620J15.3, C2orf72, RHOQ, PRPS1, ITGB8, SH3BP2, MAP3K5, PPFIA1, PLXNB1, TMEM205, ARNT2, LRPAP1, PITPNC1, MSRB2, BCKDHB, CARD19, FLNA, HRSP12, ITPKB, SLC16A9, MRPS14, TAPBP, IQCK, SDCCAG8, TKT, MAPKAPK3, NINJ1, PPIC, MARVELD1, WASF2, TRIP6, GRN, DENND5A, GLUD1, HMGCS1, GNPTG, PDLIM3, NSMF, PPA2, UROD, NRBP2, IFT22, SAP30BP, ABAT, GAB1, MSN, MIF4GD, AKR7A2, ATF3, TIMMDC1, IL6ST, SYNM, C16orf74, RFTN2, OSBPL11, CTSB, STAT3, PSMB8, MOCS2, FAM171A1, WDR1, TCTN1, SLCO1C1, FGFR3, C1QL1, GALK1, PSMB9, ARHGAP12, ITGA7, SNCAIP, TMEM179B, WWC1, MRPS28, APOA1BP, HIBCH, DNALI1, GYG1, CREM, PALLD, FAM134B, CTD-2336O2.1, GAN, CD151, STXBP3, SEPTI, HSD17B8, CNP, MPV17, GSTK1, TMED7, TRAPPC6A, ACOT13, SAR1B, RHBDD2, PHYH, ZDHHC2, CPNE2, NNT-AS1, ARL8B, RAB9A, MRC2, CCNL1, AXL, IFT43, NIPSNAP3A, BCAP31, FIGN, HIPK2, MRPS6, PIR, RPL22L1, AP006222.2, CHCHD10, FMN2, LRTOMT, MSMO1, ARHGEF10L, AKTIP, SMOX, SORBS1, SPON1, SSFA2, RIT1, LYPLAL1, KLHL5, LHFP, OXA1L, G6PC3, NACC2, SAMD8, PRSS23, CBY1, TRPS1, EVI5, SFXN5, RSU1, CYHR1, SLC25A26, CAPN2, SALL2, DHRS4-AS1, RBM38, CCS, CH17-340M24.3, MARCH2, MTSS1L, TMEM107, PRAF2, PEX2, RMDN3, PDXK, RASSF4, YAP1, CASK, FAM69C, ALG14, CPEB2, SLC6A8, ROBO3, SH3GLB1, MBNL2, PSPH, SPRY2, TMEM170A, TAB2, CD58, PCBD1, NECAP2, TSPAN6, RHPN1, C11orf49, ERBB2, DPCD, PRTFDC1, UBXN11, CTSF, EMID1, LINC00116, HSDL2, VCL, LAP3, STARD7, IMPA1, RP11-263K19.4, LPP, BMPR1B, GPR37L1, ASTN1, FMNL2, P4HTM, BBS2, SMAD1, AP2B1, SPG20, NEK6, SLC40A1, DYNC2LI1, FBXO30, ARL8A, EEPD1, YIF1B, MAGT1, TWF1, HSD17B4, WASL, ATP6V1C1, NKAIN4, KCNJ10, NPEPPS, MFHAS1, IFT57, RP3-325F22.5, CDS2, PTPRF, HHLA3, MYL5, FAM199X, SPATA20, SEPN1, TPP1, TTYH2, NMD3, FAT1, COL6A1, SUCLG2, MPDZ, LMBRD1, C5orf56, FOXK1, CAST, HOMER3, RAB29, PAQR8, CTSD, CMBL, AMFR, RNF141, ABCD3, RAB21, HS6ST1, TMED5, RENBP, TMED1, MEGF8, TOM1L2, HMGN5, FBXO8, HEATR5A, RGL2, C2orf76, ARAP2, SWI5, NT5C, LTBP1, ACBD5, SEMA6A, NAV2, S1PR1, SLC12A4, HSCB, PTRH1, FAM174A, B9D1, EFCAB14, VEPH1, TJP1, ZDHHC12, TMEM50B, TFPI, CYB5D2, LIPA, BMP7, AGTRAP, CDC42EP1, IVD, AGGF1, ANAPC10, HABP4, BTBD17, HAGHL, SGSM2, CDK2AP2, CNPY4, DMD, METTL7A, WNK1, PIK3C2A, MTTP, METTL15, CTSA, ARHGEF6, VWA3B, COL11A1, ITGAV, PHYKPL, RNF213, HEG1, GMPR2, NOTCH2, RFX3, DNASE2, RP11-140K17.3, ACP2, ALDH6A1, LRRFIP2, MPP5, TRIL, SNAP23, FAM120A, PRKD1, SALL1, TAF13, ANTXR1, CERS1, TMEM42, NUCB1, UBTD1, RGCC, TMEM189, CERS4, CYFIP1, DENND6B, FBXW9, CABIN1, VEGFB, SDSL, HS2ST1, SHISA4, DNAJC10, REST, CCDC144A, SLC27A5, EEA1, ORMDL2, DLG5, SLC4A4, SC5D, UNC5B, RCAN1, NF1, BHLHE40, LAMA4, FKBP9, LIX1L, DCAKD, GEM, CAMK2D, RHOG, NAT8L, CMTM3, PROS1, LMO2, TANC2, CSRNP1, MAPK4, AGPAT5, VAT1, AGPAT3, SCRN2, SCRIB, ZMAT1, PTGR2, ANKRD9, PAWR, OSBPL1A, COL4A1, LHPP, GSTM4, AGL, Cl4orf159, PPP1R16A, TMEM131, SNX13, IQGAP1, RB1, DACH1, COL4A5, PDE4B, ATG4C, SLC25A1, ADGRA3, SHROOM3, MMAB, ORAI3, ARHGEF10, TNIP2, SH3PXD2B, PHKG1, RP11-849119.1, TYRO3, GSTZ1, ALG13, CTDSP1, GNB4, C9orf3, APCDD1, CISD3, APBB2, CASC10, LINC01184, ERF, FBXL7, CAHM, HEY1, KANK1, FAM135A, FRS2, SLC25A23, KAT2B, IMPACT, FZD7, B4GALNT4, SIL1, ARVCF, B3GAT1, TRIM56, RPP25L, C21orf2, PEAK1, GNS, PREX1, KAZN, SPATA6, MAP4K3, PITPNA, HPS1, FASN, MAML2, KIAA1033, TCF7L2, MCCC2, ADCK4, KIAA1958, TMEM150A, SFT2D2, ARHGEF4, BMP2K, PCYT2, CCDC159, ZDHHC24, SNX21, PPP2R5A, ARHGEF40, MFSD1, NXT2, SPARCL1, TIPARP, PTDSS2, KLHDC8B, TEAD1, TMEM170B, ZBTB33, LINC00467, MMS19, BACE1, LRFN4, LSS, SLC11A2, GPC6, PHLPP1, PIPDX, GPC4, RYK, LNPEP, DESI1, NLGN3, and SOAT1.

In some embodiments, a radial glia in an organoid cultured for at least 6 months or more is characterized as an organoid cell that overexpresses, as compared to the rest of the organoid cells, at least 1, 2, 3, 4, 5, 6, 7, 8, 9, 10, 11, 12, or all 13 of the following genes: ADM, IGFBP2, AK4, IGFBP5, TGIF1, PTPRZ1, PMP2, SFRP1, PRDX4, PGM1, HES1, SERPINE2, and RGS16.

In some embodiments, an outer radial glia in an organoid cultured for at least 6 months or more is characterized as an organoid cell that overexpresses, as compared to the rest of the organoid cells, at least 5, 10, 20, 30, 40, 50, 75, 100, 125, 150, 175, 200, 250, 300, 350, 400, 450, 500, or all 512 of the following genes: MT3, C8orf4, ATP1A2, CDO1, CA2, TTYH1, APOE, PEA15, LRRC3B, MLC1, REXO2, PTN, PON2, SLC1A3, TRIM9, TNC, BCAN, PTPRZ1, METRN, CST3, CLU, SCRG1, QKI, ITM2C, VIM, HMGN3, GPM6B, TSPAN3, HOPX, MGST3, BAALC, AQP4, B2M, ITM2B, DDAH1, SNX3, INPP1, ADGRV1, ATP1B2, TSC22D4, DOK5, CSPG5, HIGD1A, ID4, HTRA1, BST2, SEPW1, EDNRB, OAT, HSD17B14, ENHO, SDC2, PLPP3, PSAP, CROT, SAT1, GCSH, TFPI, PBXIP1, MGLL, LITAF, NPC2, BDH2, TMEM132B, SPAG16, ZFHX4, PMP22, ADD3, TIMP2, LSAMP, TMEM47, PDGFRB, CHPF, CYSTM1, DNPH1, PAQR8, DDR1, HES5, TP53TG1, ACYP2, HADHB, PLA2G16, IL33, ABHD3, IGFBP7, ANXA6, NDRG4, ANXA5, FZD8, EEPD1, SLC25A18, HLA-A, NFE2L2, LIMCH1, OBSL1, HSPB1, PLEKHB1, LGALS3BP, PRDX6, C1orf122, RHOA, CHCHD10, C1orf6l, LINC00982, CHPT1, IFI27L2, SPATS2L, DTNA, PDLIM3, CD99, HIGD2A, CD58, UQCR11, F3, FAM107A, GPR137B, SARAF, CYBA, LTBP1, BLVRB, PDLIM5, ADGRG1, NOTCH2, LAMP1, GADD45A, SPTSSA, SCRN1, RHOC, LYRM5, SERPINB6, GNG5, OAF, MTCO1, RAB6B, PAQR6, LAMTOR4, SALL1, C4orf3, NDUFB5, DKK3, GLUD1, TMEM9B, PPT1, POLR2L, QPRT, FAM69C, REEP5, PAM, LHX2, COX6C, DPP7, S1PR1, VAT1L, BMP7, HSD17B12, COMT, CTSL, WASF3, TLE4, PRDM16, LINC00998, FAM198B, CHMP4B, CSTB, PIR, HEBP1, TMEM132A, TIMP1, SFXN5, COL11A1, ELOVL5, TAGLN2, MYO6, AXL, HLAE, DHRS4L2, CEND1, HLA-B, HRSP12, GLI3, MSRB2, CYR61, FKBP9, APLP2, FAM3C, C1S, GNAS, FGFR3, RAB31, IGFBP4, RP11-263K19.4, SLCO1C1, TIMP3, SNX5, LTBP3, GPX3, FERMT2, MYEOV2, ACADVL, BORCS7, UBL3, MAPKAPK3, PTGFRN, HLA-C, MRC2, CISD1, NEAT1, ACAA1, SLC9A3R1, LRIG1, ANKRD9, CD164, PGM1, SYT11, FOSB, NPAS3, SQSTM1, PLTP, AIG1, SELT, SPATA20, STXBP3, CEBPD, NDUFA13, SLC35F1, NDUFB3, RAMP1, MPP5, PNKD, YAP1, ITGB8, B4GAT1, LAMB2, ARAP2, ZFYVE21, RP3-325F22.5, SPRY2, HDDC2, BCAP29, SDHC, C16orf74, DAG1, LRP4, NDUFB1, FGFR1, LAMP2, TFAP2C, HAGHL, HES4, PCBD1, FAT1, CREM, TMED10, TMEM163, SALL2, LRP10, ATP6V0E1, FOXK1, SEMA5A, CD81, NAA38, HINT2, MYL12A, DAAM2, VAMP5, CRYL1, PDLIM2, OLFM2, ROMO1, GAS2L1, PCGF5, TMED1, KLHDC9, MMP15, TRAPPC6A, TPP1, NME3, GPR37L1, PDHB, SDSL, GSTK1, PPP2CB, FEZ2, GULP1, SEMA6A, KTN1, NAT8L, SFT2D1, C1orf54, LRP1, MMP14, SHROOM3, TM7SF2, RP11-431M7.3, ITPR2, COPRS, CLTC, ST5, ATP1A1, MOXD1, MAPK1, NEK6, CYHR1, LPAR4, SORL1, NRG1, ASAH1, QDPR, C2orf72, P4HTM, CTD-2336O2.1, CDH4, ZMAT3, ASTN1, GAB1, NT5C, SCARA3, ISCA2, NRBP2, EMID1, LIFR, CNP, EPDR1, TMEM98, TP53I3, TCEAL3, MRPS28, FAM199X, DECR1, RNF213, TMEM59L, GNPTG, GSTO1, TAPBP, THSD1, GEM, CA12, UG0898H09, ITGAV, RIT1, RHPN1, B9D1, CREB5, EFEMP2, ZDHHC2, JAM3, DENND5A, ITGA6, PRCP, PHLPP1, ABCD3, RHPN2, GNG12, GPC6, TSPAN6, CH17-189H20.1, VEGFB, KAZN, PLCD3, METTL7B, MPV17, COL4A5, LPP, MIF4GD, TMEM134, USF2, LIX1L, HEATR5A, PPP2R5A, TRIP6, NQO1, CTD-3252C9.4, CHCHD5, FAM213A, ROM1, SCD, ATP6V1C1, PEX2, TAF13, TMEM179B, DNASE2, GRN, PLCE1, SDC3, MYL5, RARRES3, PRUNE2, TMED5, SPARC, WDR41, NACC2, BICD1, RHOQ, PRKD1, FAM84B, FAM173A, ADAM9, NDP, UBTD1, RENBP, PTPMT1, RFXANK, SGSM2, SSFA2, IMPA1, GRIN2A, ACP2, COA5, TTYH3, RAB9A, REST, S100A16, AHNAK, TMBIM4, PVRL2, MMP24-AS1, CDC42EP1, PDZD11, SOAT1, ADGRB2, MORN2, SLC20A1, CTSD, CTSB, GLIPR2, FADS2, SLC27A1, MAGT1, MOCS2, TMEM205, RP11-410L14.2, C21orf62, CCL2, B3GAT1, PSMB8, ACAT2, AIF1L, ARRDC4, CAST, UROD, DNAJC1, PEPD, PRNP, RP11-140K17.3, CARD19, ACTN1, SCRIB, CAMK2D, HEXB, GLUL, SLC2A8, S100A13, PDXK, IVD, RASSF4, PAWR, PLEC, PLAT, L3HYPDH, SHISA4, PEX10, KRCC1, MSN, ANOS1, TNFRSF1A, NIPSNAP3A, CISD3, SLC16A9, TNFRSF12A, RRAGD, IRS2, COLGALT2, CTSA, WLS, RGS20, SLC27A5, INPPL1, LMO2, SPARCL1, ERF, SLC44A2, NUDT22, SMPD1, NRCAM, RGS3, SWI5, FAM84A, SLC35F5, GLB1, AGTRAP, CFI, RAB29, RGL2, TRIB2, ZDHHC12, HS2ST1, PREX1, ID1, SREBF2, ID3, OSGIN2, SEL1L3, IL6ST, REEP3, CH17-340M24.3, CD44, SIPA1L1, RCAN1, H2AFJ, HABP4, EFHD2, and GLMP.

In some embodiments, an outer radial glia/astroglia in an organoid cultured for about 6 months or more is characterized as an organoid cell that overexpresses, as compared to the rest of the organoid cells, at least 5, 10, 20, 30, 40, 50, 75, 100, 125, 150, 175, 200, 250, 300, 350, 400, 450, 500, 550, or all 562 of the following genes: CRYAB, NCAN, BCAN, VIM, HES1, PRDX6, CLU, SAT1, TRIM9, TTYH1, SOX3, AQP4, SOX9, METRN, LIMCH1, HMGN3, TSC22D4, ID4, CDO1, DOK5, RFXANK, ID1, C1orf6l, AIF1L, PEA15, IGFBP5, EZR, LRRC3B, QKI, ZFHX4, ZFP36L1, PTPRZ1, MYO10, GCA, CNN3, NOTCH2, ADGRV1, TIMP2, GLI3, CYBA, GTF2F2, RHOC, PHYHIPL, HSPB1, LIPG, CYR61, CTSH, IFI27L2, DDAH1, GFAP, SLC35F1, ANXA1, SCRN1, PDLIM4, FAM84B, TSPAN3, FSTL1, NPC2, MLC1, HIGD1A, FAM107A, ALDOC, SCRG1, LITAF, SEC11A, PSAT1, GCSH, CSTB, NME4, SOX6, CSPG5, SH3BGRL, CAMTA1, LINC00998, CHCHD5, FEZ2, ATP1B2, TM7SF2, HTRA1, BAALC, COMT, IFITM3, DNPH1, COLGALT2, CREB5, FAM198B, NDRG2, OXA1L, KTN1, SEMA6A, SLC25A5, NINJ1, ANXA6, CYSTM1, HIGD2A, SERF2, TKT, PDLIM2, LHX2, SLC1A3, NKAIN3, S100A16, NDUFA13, SYT11, SESN3, SLC25A18, LRIG1, PSAP, ARHGAP5, HADHB, ITGB8, RPL22L1, CD63, POLR2L, IGFBP4, BMP7, CD151, PON2, NAV1, SDCBP, APC, AKR7A2, SPAG16, TRAPPC6A, FKBP10, URM1, ATP1A2, CNP, ASPH, DAAM2, APRT, TRIP6, TAGLN2, UG0898H09, MMP24-AS1, HINT2, SLC16A9, SEPT9, GRIN2A, OAT, NT5C, GNAI2, PLPP3, C1orf122, GSTK1, OGFRL1, FLNA, PDLIM5, FGFR3, IFI44L, CST3, PAG1, PCBD1, ITM2B, GPR137B, NME3, REEP5, TMEM132B, WDR1, LAMP2, COL11A1, ST5, LSAMP, APOA1BP, CIB1, C8orf4, TP53I3, PMP22, HMGCS1, PDLIM7, GNG12, MSMO1, TMEM47, MSI1, COPRS, UQCR11, DACH1, CAMK2D, TMEM134, TFAP2C, PAQR8, LGALS3BP, BICD1, LINC00982, EEPD1, ALDH3A2, ZFP36, LPAR4, LRRC16A, KCNN3, REXO2, HES5, PRDM16, BLVRB, RP11-126K1.6, IL33, CARD19, EVA1C, UBE2H, SFRP1, TMEM179B, CA2, PALLD, GRN, MTRNR2L1, APOE, LRP10, MTTP, SLC9A3R1, NFE2L2, ENHO, MAPK1, ACAA1, ACAA2, ACOX1, ALDH2, MT-CO2, MINCR, FOXK1, SPRY2, LDB2, TPP1, PBXIP1, TOX, PEX10, LIFR, CYHR1, POLE4, SALL1, CTNND1, SLC25A39, DHRS4L2, MACROD1, PHGDH, RP11-76114.1, MYL5, TMEM131, MSN, PELI2, DNAJC1, NOTCH1, SNX17, BOC, HEY1, CRB2, HEPN1, SNX5, NACC2, MSRB2, KLHL21, FAM69C, PLTP, NDRG4, RAB31, VAMP5, P4HTM, ADD3, MMP15, ACTN1, RAB11BAS1, ERBB2IP, UBL3, RIT1, ITGA7, REEP3, ARNT2, PDLIM3, VCL, HAGHL, ABAT, ARHGAP12, TAF13, WDR6, FGFR1, TMEM170A, CDC42EP1, STON2, ARRDC4, SFXN5, METTL7A, OSGIN2, CEND1, DKK3, POLR3H, USF2, BST2, GALK1, LTBP1, SLC27A5, IVD, ADAM9, DOCK7, C21orf62, MOXD1, TMEM141, GMPR2, SSFA2, FGFR2, PHLPP1, PLCE1, SOD2, GULP1, PLCD3, GAS2L1, GEM, CTD-2336O2.1, CBY1, FAM120A, PAM, RAB6B, ROM1, ECI1, LTBP3, TTYH3, NPAS3, PTPN11, WASF3, GLUD1, PLEKHB1, PPT1, OAF, HSD17B14, HRSP12, B9D1, S100A13, MMP14, PDGFD, AXL, CREM, RP11-263K19.4, PAXIP1-AS1, CHPT1, DAG1, ACYP2, MGLL, TP53TG1, RHOQ, FBXO32, RPP25L, HOMER3, FAM134B, GSTZ1, NEK6, DENND5A, NUDT22, MAPK8IP1, HEPACAM, KLHDC8B, SERPINB6, NAT8L, SLCO1C1, RFTN2, FAM84A, IFT57, CD38, GPR37L1, BDH2, S1PR1, HIST2H2BE, ATP2B4, PDCD4, SDSL, PIR, LGALS3, SOX21, C2orf72, CITED1, TCTN1, FGFBP3, GYG1, NRG1, YAP1, FLCN, ALDH6A1, TIMP3, INSIG1, SELENBP1, FZD8, PREX1, AKR1C3, E2F5, SCRN2, TRPS1, SAMD4A, PLA2G16, SLC25A23, PLXNB1, STAT1, CTSA, MGAT4C, NADK2, IQGAP2, TMED1, NMD3, TAPBP, RAI14, CROT, BTBD17, PSMB8, INPP1, HEBP1, DUSP3, RP11-25K19.1, EPHX1, SHISA4, JAM3, OBSL1, TCF7L2, OPHN1, KCNG1, DCAF8, TANC2, KRCC1, SHC1, PPM1K, GNPTG, KCNJ10, BMP2K, KAZN, PAQR6, PTGFRN, FBXO30, FAM199X, ADAM15, ACTR3B, RP11-410L14.2, HSDL2, MID1, ARAP2, MMAB, ERF, RP11-431M7.3, ZDHHC12, DNASE2, MAPKAPK3, FAT1, PLP1, RNF213, AHNAK, PLEC, PGM1, LRRC1, NR2E1, PAWR, CTD-3252C9.4, MARVELD1, RHPN1, ZDHHC2, RAB9A, B3GALNT2, PIK3C2A, EVI5, HEATR5A, REST, SPATA6, C16orf74, IL6ST, PITPNA, S1PR3, MTSS1L, NAPEPLD, EFHC1, TMEM189, SHROOM3, CLCN7, RP11-140K17.3, LAMB2, MPP5, PVRL2, SLC7A11, PDGFRB, L3HYPDH, MAP4K3, TBC1D1, NEDD9, FKBP9, LSS, CISD3, ITGA6, CD58, SEPN1, HHLA3, SOAT1, SPRED1, MAP3K5, STXBP3, AGTRAP, MRC2, EFEMP2, SLC2A8, ASTN1, RBM38, AP1B1, ANKRD9, ZHX3, WWC1, INPPL1, CXCL12, MAGT1, RP3-325F22.5, MRPS28, ZBTB4, ABHD3, ZMAT1, SIPA1L1, MOB1B, UBTD1, ANOS1, PHF10, CCDC159, PCGF5, PPP2R5A, AEBP1, TFE3, GPC6, SGSM2, CHST3, SNTA1, FAM102A, ABHD17C, RGS3, PHYH, EFHD1, C1orf53, SYNGR1, COL4A5, WLS, SCRIB, AASS, LAMA4, PRKD1, HPS1, C5orf56, ORAI3, TMEM163, ERBB2, MBNL2, AGPAT3, NECAB3, GSTM4, SPATA20, ACP6, NR1D2, KLHDC9, LRRCC1, CTDSP1, PDPN, PHKG1, AAED1, EMID1, LRTOMT, C6orf120, MFSD14B, APBB2, CYBRD1, C1orf194, CNPY4, SNX21, VCAM1, NRBP2, FNDC3B, and TFPI.

In some embodiments, the organoid has been cultured for about 12 months or more and comprises from about 6% to about 16% astroglia, from about 7% to about 22% callosal projection neurons, from about 5% to about 8% cycling progenitors, from about 10% to about 31% immature interneurons, from about 2% to about 10% immature projection neurons, from about 1% to about 7% intermediate progenitor cells, from about 22% to about 39% radial glia, and from about 4% to about 8% ventral precursors.

In some embodiments, corticofugal projection neurons are characterized as cells expressing BCL11B, CRYM, and TLE4 marker genes. In some embodiments, callosal projection neurons are characterized as cells expressing SATB2, INHBA, and FRMD4B marker genes. In some embodiments, interneurons are characterized as cells expressing DLX1, DLX2, and GAD2 marker genes. In some embodiments, outer radial glia are characterized as cells expressing HOPX, TNC and LGALS3 marker genes. In some embodiments, intermediate progenitor cells are characterized as cells expressing EOMES, PPP1R17, and TMEM158 marker genes. In some embodiments, cycling precursors are characterized as cells expressing MKI67, TOP2A, and BIRC5 marker genes.

The organoid may be derived cells of a mammalian species, e.g., human, equine, bovine, porcine, canine, feline, rodent, primate, etc. In some examples, the organoid is derived human cells. In some examples, the organoid is derived from rodent cells (e.g., mouse cells, rat cells). In a particular example, the organoid is a human dorsal forebrain organoid. In another example, the organoid is a mouse dorsal forebrain organoid. In another example, the organoid is a rat dorsal forebrain organoid.

### Glioma Cells

The compositions and systems may further comprise one or more brain tumor cells such as glioma cells. The glioma cells may be from an established glioma cell line. In certain embodiments, the glioma cells may be derived from a subject suffering from a glioma or may be cells derived from a subject suffering from a glioma that have been cultured prior to being added to the organoid model. In some cases, the glioma cells in the composition may be cells implanted into the organoid. Alternatively or additionally, the glioma cells in the compositions may be progeny of or derived from cells implanted into the organoid.

A glioma cell refers to a cell of or derived from a glioma. A glioma refers to a type of cancer arising from glial cells (e.g., in the brain or spine). A glial cell refers to a cell that surrounds neurons and provides support for and insulation between them. Glial cells are the most abundant cell types in the central nervous system. Types of glial cells include oligodendrocytes, astrocytes, ependymal cells, Schwann cells, microglia, and satellite cells. Oligodendrocytes are neural cells of ectodermal origin, forming part of the adventitial structure (neuroglia) of the central nervous system. They have variable numbers of veil-like or sheet-like processes that wrap around individual axons to form the myelin sheath of the CNS. They can be identified by morphological, phenotypic, or functional criteria as explained later in this disclosure. Astrocytes are specialized glial cells that outnumber neurons by over fivefold. They contiguously tile the entire central nervous system (CNS) and exert many essential complex functions in the healthy CNS. Astrocytes respond to all forms of CNS insults through a process referred to as reactive astrogliosis, which has become a pathological hallmark of CNS structural lesions.

Glioma herein include those classified by cell type, by tumor grade, and by location. Examples of glioma include ependymomas, astrocytomas (e.g., glioblastoma multiforme), and oligodedrogliomas, mixed gliomas (e.g., comprising cells from different types of glia, such as oligoastrocytomas), a supratentorial glioma (e.g., located above the tentorium), an infratentorial glioma (e.g., located below the tentorium), diffuse intrinsic pontine glioma (DIPG), thalamic glioma, gliobastoma multiforme, ependymoma, astrocytoma, oligodendroglioma, optic nerve glioma, choroid plexus papilloma, and spinal cord glioma. In some examples, glioma may be grade IV glioblastoma, high grade pediatric glioma, diffuse intrinsic pontine glioma (DIPG), or isocitrate dehydrogenase (IDH) mutant glioma. In some examples, glioma may be IDH-wild type primary glioblastoma, IDH-mutant astrocytoma, or IDH-mutant oligodendroglioma. In an example, the glioma is glioblastoma.

In some cases, glioma cells implanted may be patient-derived glioma cells. For example, the glioma cells can originate from human patient-derived glioma cells implanted into the organoid. Patient-derived glioma cells include cells from glioma in patients or progeny thereof. The patient-derived glioma cells may be cells derived from patients with glioma described herein. Examples of patient-derived cells also include those described in David P. Kodack et al., Primary Patient-Derived Cancer Cells and Their Potential for Personalized Cancer Patient Care, Cell Rep. 2017 Dec. 12; 21(11): 3298-3309.

The glioma cells may comprise one or more stages of cells. In some embodiments, a glioma cell may transition to a different type of glioma cell after implantation. For example, the glioma cells (before or after implantation) may comprise one or more of oligodendrocyte progenitor cell (OPC)-like, astrocyte (AC)-like, neural progenitor cell (NPC)-like, oligodendroglioma cell (OC)-like, or mesenchymal cell (MES)-like cells. In one example, the glioma cell (before or after implantation) comprises one of OPC-like cells, AC-like cells, NPC-like cells, OC-like cells, or MES-like cells. In one example, the glioma cell (before or after implantation) comprises two of OPC-like cells, AC-like cells, NPC-like cells, OC-like cells, or MES-like cells. In one example, the glioma cell (before or after implantation) comprises three of OPC-like cells, AC-like cells, NPC-like cells, OC-like cells, or MES-like cells. In one example, the glioma cell (before or after implantation) comprises all of OPC-like cells, AC-like cells, NPC-like cells, OC-like cells or MES-like cells.

The following tables identify genes expressed at certain stages of human patient-derived glioma cells (e.g., DIPG cells, IDH-wild type primary glioblastoma cells, IDH-mutant astrocytoma cells, or IDH-mutant oligodendroglioma cells). For example, the tables identify the genes expressed by AC-like cells, NPC-like cells, OC-like cells, OPC-like cells, and MES-like cells in different glioma cells.

TABLE 1
Gene expression signatures in IDH-WT glioblastoma. The table identifies those genes whose average
log-ratios were above 2 and was restricted to the top 50 genes with highest log-ratios for that
group of signatures. Genes are listed in descending order according to these average log ratios.

| MES2 | MES1 | AC | OPC | NPC1 | NPC2 | G1/S | G2/M |
|---|---|---|---|---|---|---|---|
| HILPDA | CHI3L1 | CST3 | BCAN | DLL3 | STMN2 | RRM2 | CCNB1 |
| ADM | ANXA2 | S100B | PLP1 | DLL1 | CD24 | PCNA | CDC20 |
| DDIT3 | ANXA1 | SLC1A3 | GPR17 | SOX4 | RND3 | KIAA0101 | CCNB2 |
| NDRG1 | CD44 | HEPN1 | FIBIN | TUBB3 | HMP19 | HIST1H4C | PLK1 |
| HERPUD1 | VIM | HOPX | LHFPL3 | HES6 | TUBB3 | MLF1IP | CCNA2 |
| DNAJB9 | MT2A | MT3 | OLIG1 | TAGLN3 | MIAT | GMNN | CKAP2 |
| TRIB3 | C1S | SPARCL1 | PSAT1 | NEU4 | DCX | RNASEH2A | KNSTRN |
| ENO2 | NAMPT | MLC1 | SCRG1 | MARCKSL1 | NSG1 | MELK | RACGAP1 |
| AKAP12 | EFEMP1 | GFAP | OMG | CD24 | ELAVL4 | CENPK | CDCA3 |
| SQSTM1 | C1R | FABP7 | APOD | STMN1 | MLLT11 | TK1 | TROAP |
| MT1X | SOD2 | BCAN | SIRT2 | TCF12 | DLX6-AS1 | TMEM106C | KIF2C |
| ATF3 | IFITM3 | PON2 | TNR | BEX1 | SOX11 | CDCA5 | AURKA |
| NAMPT | TIMP1 | METTL7B | THY1 | OLIG1 | NREP | CKS1B | CENPF |
| NRN1 | SPP1 | SPARC | PHYHIPL | MAP2 | FNBP1L | CDC45 | KPNA2 |
| SLC2A1 | A2M | GATM | SOX2-OT | FXYD6 | TAGLN3 | MCM3 | KIF20A |
| BNIP3 | S100A11 | RAMP1 | NKAIN4 | PTPRS | STMN4 | CENPM | ECT2 |
| LGALS3 | MT1X | PMP2 | LPPR1 | MLLT11 | DLX5 | AURKB | BUB1 |
| INSIG2 | S100A10 | AQP4 | PTPRZ1 | NPPA | SOX4 | PKMYT1 | CDCA8 |
| IGFBP3 | FN1 | DBI | VCAN | BCAN | MAP1B | MCM4 | BUB1B |
| PPP1R15A | LGALS1 | EDNRB | DBI | MEST | RBFOX2 | ASF1B | TACC3 |
| VIM | S100A16 | PTPRZ1 | PMP2 | ASCL1 | IGFBPL1 | GINS2 | TTK |
| PLOD2 | CLIC1 | CLU | CNP | BTG2 | STMN1 | MCM2 | TUBA1C |
| GBE1 | MGST1 | PMP22 | TNS3 | DCX | HN1 | FEN1 | NCAPD2 |
| SLC2A3 | RCAN1 | ATP1A2 | LIMA1 | NXPH1 | TMEM161B-AS1 | RRM1 | ARL6IP1 |
| FTL | TAGLN2 | S100A16 | CA10 | HN1 | DPYSL3 | DUT | KIF4A |
| WARS | NPC2 | HEY1 | PCDHGC3 | PFN2 | SEPT3 | RAD51AP1 | CKAP2L |
| ERO1L | SERPING1 | PCDHGC3 | CNTN1 | SCG3 | PKIA | MCM7 | MZT1 |
| XPOT | C8orf4 | TTYH1 | SCD5 | MYT1 | ATP1B1 | CCNE2 | KIFC1 |
| HSPA5 | EMP1 | NDRG2 | P2RX7 | CHD7 | DYNC1I1 | ZWINT | SPAG5 |
| GDF15 | APOE | PRCP | CADM2 | GPR56 | CD200 | | ANP32E |
| ANXA2 | CTSB | ATP1B2 | TTYH1 | TUBA1A | SNAP25 | | KIF11 |
| EPAS1 | C3 | AGT | FGF12 | PCBP4 | PAK3 | | PSRC1 |
| LDHA | LGALS3 | PLTP | TMEM206 | ETV1 | NDRG4 | | TUBB4B |
| P4HA1 | MT1E | GPM6B | NEU4 | SHD | KIF5A | | SMC4 |
| SERTAD1 | EMP3 | F3 | FXYD6 | TNR | UCHL1 | | MXD3 |
| PFKP | SERPINA3 | RAB31 | RNF13 | AMOTL2 | ENO2 | | CDC25B |
| PGK1 | ACTN1 | PPAP2B | RTKN | DBN1 | KIF5C | | OIP5 |
| EGLN3 | PRDX6 | ANXA5 | GPM6B | HIP1 | DDAH2 | | REEP4 |
| SLC6A6 | IGFBP7 | TSPAN7 | LMF1 | ABAT | TUBB2A | | FOXM1 |
| CA9 | SERPINE1 | | ALCAM | ELAVL4 | LBH | | TMPO |
| BNIP3L | PLP2 | | PGRMC1 | LMF1 | LOC150568 | | GPSM2 |
| RPL21 | MGP | | HRASLS | GRIK2 | TCF4 | | HMGB3 |
| TRAM1 | CLIC4 | | BCAS1 | SERINC5 | GNG3 | | ARHGAP11A |
| UFM1 | GFPT2 | | RAB31 | TSPAN13 | NFIB | | RANGAP1 |
| ASNS | GSN | | PLLP | ELMO1 | DPYSL5 | | H2AFZ |
| GOLT1B | NNMT | | FABP5 | GLCCI1 | CRABP1 | | |
| ANGPTL4 | TUBA1C | | NLGN3 | SEZ6L | DBN1 | | |
| SLC39A14 | GJA1 | | SERINC5 | LRRN1 | NFIX | | |
| CDKN1A | TNFRSF1A | | EPB41L2 | SEZ6 | CEP170 | | |
| HSPA9 | WWTR1 | | GPR37L1 | SOX11 | BLCAP | | |

TABLE 2
Gene Expression Signatures in DIPG (H3K27M-Glioma).

| Cellcycle | OC | AC | OPC-shared | OPC-variable |
|---|---|---|---|---|
| UBE2T | BCAS1 | AQP4 | PDGFRA | PDGFRA |
| HMGB2 | PLP1 | CLU | MEST | ITM2C |
| TYMS | PTGDS | AGT | CCND1 | SCG3 |
| MAD2L1 | GPR17 | SPARCL1 | KLRC2 | SERPINE2 |
| CDK1 | TUBB4A | VIM | ARC | CSPG4 |
| UBE2C | MBP | CRYAB | SEZ6L | CA10 |
| RRM2 | TF | GFAP | EGR1 | PTPRZ1 |
| PBK | SIRT2 | APOE | CD24 | CNTN1 |
| ZWINT | FYN | MLC1 | ASCL1 | NAV1 |
| NUSAP1 | MOG | EDNRB | FOS | TNR |
| PCNA | CNP | GJA1 | LINC00643 | LRP1 |
| BIRC5 | NFASC | SPON1 | ETV1 | TSPAN7 |
| H2AFZ | BMPER | PLTP | NNAT | SEMA5A |
| FAM64A | MPZL1 | ALDOC | EGR2 | CST3 |
| TOP2A | RGR | HSPB8 | PCP4 | GPM6A |
| KIAA0101 | CLDN11 | HEY1 | BTG2 | COL9A1 |
| PTTG1 | TNFRSF21 | DAAM2 | HES6 | APOD |
| GMNN | GNAI1 | TNC | IER2 | SLC1A2 |
| KPNA2 | TMEM206 | S1PR1 | MFNG | SPRY4 |
| TUBA1B | TMOD1 | TIMP3 | | NLGN3 |
| NUF2 | RAB33A | EZR | | C3orf70 |
| TPX2 | SGK1 | SPARC | | CHAD |
| MLF1IP | TNR | SLC1A3 | | PSAP |
| HIST1H4C | TMTC4 | PON2 | | ZCCHC24 |
| KIF22 | FDFT1 | ATP1A2 | | EPN2 |
| TMPO | WASF1 | HLA-C | | DPYSL2 |
| CKS2 | ZNF488 | PSAT1 | | GPRC5B |
| CDCA5 | UGT8 | TGFBI | | TRIB2 |
| CENPM | BIN1 | CXCR4 | | BCAN |
| PRC1 | SEMA6D | CD99 | | ITM2B |
| MCM7 | APLP1 | EEPD1 | | ABHD2 |
| TMSB15A | EPB41L2 | SFRP2 | | LHFPL3 |
| CENPF | DYNLL1 | NID1 | | CHL1 |
| RNASEH2A | KANK1 | S100A16 | | GPM6B |
| RACGAP1 | TNS3 | C2orf40 | | MEG3 |
| DUT | SCRG1 | CCDC80 | | NXPH1 |
| CKS1B | DBNDD2 | ID4 | | PLEKHB1 |
| AURKB | CADM1 | B2M | | LNX1 |
| CCNB2 | IGSF11 | ITM2C | | HMP19 |
| DTL | PLXNB3 | KAL1 | | EDIL3 |
| FEN1 | PFN2 | HLA-B | | GRIA2 |
| FANCI | LRRN3 | F3 | | B3GNT7 |
| KIF11 | TSPAN15 | PBXIP1 | | HLA-C |
| RRM1 | SEMA5B | CDC42EP4 | | CD9 |
| MCM2 | APCDD1 | CST3 | | SYT11 |
| CDC20 | PSAT1 | GLUD1 | | ATP6AP2 |
| HMGN2 | E2F3 | CD44 | | XYLT1 |
| CCNA2 | ARHGAP5 | TTYH1 | | ACSL3 |
| TK1 | PKP4 | S100A10 | | GNG7 |
| PKMYT1 | KIF21A | BTBD17 | | EPAS1 |

TABLE 3
Gene expression signatures in IDH-mutant astrocytoma.

| Oligo-program | Astro-program | Stemness program |
|---|---|---|
| OLIG1 | APOE | SOX4 |
| NEU4 | SPARCL1 | DCX |
| GPR17 | VIM | IGFBPL1 |
| SLC1A1 | ID4 | SOX11 |
| ATCAY | TIMP3 | TCF4 |
| SIRT2 | EDNRB | NREP |
| APOD | MLC1 | RND3 |
| MYT1 | ID3 | CCND2 |
| OLIG2 | CLU | MIAT |
| TMEFF2 | TNC | CAMK2N1 |
| OMG | ZFP36L1 | STMN4 |
| ELMO1 | ARHGEF26 | STMN1 |
| RTKN | ATP1B2 | MYT1L |
| HIP1R | AGT | HN1 |
| TNR | RGMA | RNF122 |
| RPSA | JUN | PROX1 |
| MEGF11 | PFKFB3 | KLHDC8A |
| EVI2A | EZR | ELAVL4 |
| OPCML | SLC1A3 | NMNAT2 |
| LHFPL3 | ALDOC | TUBB |
| RAB33A | JUNB | ROBO1 |
| GRIA4 | ATP1A2 | NELL2 |
| SERINC5 | DTNA | MLLT11 |
| NXPH1 | ZFP36 | CELF4 |
| BIN1 | SOX9 | POU3F2 |
| BMP4 | TRIL | H3F3B |
| EHD3 | NDRG2 | ENC1 |
| GNAI1 | NMB | GNG2 |
| CSPG4 | GFAP | ACOT7 |
| DSCAM | SLC1A2 | AKT3 |
| GALNT13 | RFX4 | ARL4C |
| ZDHHC9 | MALAT1 | FNBP1L |
| ABCG1 | LRIG1 | VOPP1 |
| FKBP1A | FOS | TOX3 |
| LRRN1 | EGR1 | TUBB3 |
| ST8SIA3 | STK17B | SCG2 |
| DNM3 | FOSB | TMSB15A |
| RAPGEF4 | ATF3 | TFDP2 |
| CNP | ABCA1 | TMSB4X |
| PDGFRA | ADCYAP1R1 | CDC42 |
| PTGDS | GLUL | STMN2 |
| CHGA | IER2 | KCTD13 |
| BCAS1 | ZFP36L2 | RPH3A |
| PLXNB3 | ADHFE1 | KIF5C |
| NFASC | MSI2 | NFIX |
| SLC44A1 | CPE | CALM1 |
| GNG4 | KLF6 | TNPO2 |
| PHLDB1 | DOCK7 | BOC |
| CD82 | IRF2BP2 | KLHL13 |
| PRKCZ | SPRY2 | PGAP1 |
| | | RBFOX2 |
| | | TMSB10 |
| | | DYNLT1 |
| | | TMSB15B |
| | | TCEAL7 |
| | | PTS |
| | | BICD1 |
| | | UCHL1 |
| | | COMMD3 |
| | | MCM7 |
| | | AMZ2 |
| | | PDRG1 |
| | | DDAH2 |
| | | KLC1 |
| | | PCSK2 |
| | | OAZ1 |
| | | TIMM17A |
| | | YWHAG |
| | | CBX1 |
| | | SMS |
| | | DGUOK |
| | | SNRPG |
| | | CDK6 |
| | | GOLT1B |
| | | DUSP10 |
| | | ATP5J |
| | | DYNLRB1 |
| | | TCP1 |
| | | GADD45G |
| | | SEC31A |
| | | CNOT7 |
| | | DDX39A |
| | | SRGAP2 |
| | | MAST2 |
| | | PGK1 |
| | | CELF3 |
| | | ZFAS1 |
| | | ENO2 |
| | | SNRPB |
| | | DRG1 |

TABLE 4
Gene expression signatures in IDH-mutant oligodendroglioma. Each gene set is ranked from most significant
(top) to least significant gene (bottom). Significance was determined by average fold-change of upregulation
in G1/S, G2/M and stem-like cells (first three columns) or by the correlation with PC1 (positive correlation
for OC genes and negative for AC genes). Two gene sets are given for each of the lineages: “PCA-only”
denotes genes that were identified from PCA analysis of oligodendroglioma cells and “PCA + mice”
denotes genes that were both identified in the PCA analysis of oligodendroglioma cells and are preferentially
expressed in the resective lines in mice, and these were used to estimate lineage scores.

| | | | AC | AC | OC | OC |
|---|---|---|---|---|---|---|
| G1/S | G2/M | stemness | (PCA-only) | (PCA + mice) | (PCA-only) | (OG + mice) |
| MCM5 | HMGB2 | SOX4 | APOE | APOE | LMF1 | OLIG1 |
| PCNA | CDK1 | CCND2 | SPARCL1 | SPARCL1 | OLIG1 | SNX22 |
| TYMS | NUSAP1 | SOX11 | SPOCK1 | ALDOC | SNX22 | GPR17 |
| FEN1 | UBE2C | RBM6 | CRYAB | CLU | POLR2F | DLL3 |
| MCM2 | BIRC5 | HNRNPH1 | ALDOC | EZR | LPPR1 | SOX8 |
| MCM4 | TPX2 | HNRNPL | CLU | SORL1 | GPR17 | NEU4 |
| RRM1 | TOP2A | PTMA | EZR | MLC1 | DLL3 | SLC1A1 |
| UNG | NDC80 | TRA2A | SORL1 | ABCA1 | ANGPTL2 | LIMA1 |
| GINS2 | CKS2 | SET | MLC1 | ATP1B2 | SOX8 | ATCAY |
| MCM6 | NUF2 | C6orf62 | ABCA1 | RGMA | RPS2 | SERINC5 |
| CDCA7 | CKS1B | PTPRS | ATP1B2 | AGT | FERMT1 | LHFPL3 |
| DTL | MKI67 | CHD7 | PAPLN | EEPD1 | PHLDA1 | SIRT2 |
| PRIM1 | TMPO | CD24 | CA12 | CST3 | RPS23 | OMG |
| UHRF1 | CENPF | H3F3B | BBOX1 | SOX9 | NEU4 | APOD |
| MLF1IP | TACC3 | C14orf23 | RGMA | EDNRB | SLC1A1 | MYT1 |
| HELLS | FAM64A | NFIB | AGT | GABRB1 | LIMA1 | OLIG2 |
| RFC2 | SMC4 | SRGAP2C | EEPD1 | PLTP | ATCAY | RTKN |
| RPA2 | CCNB2 | STMN2 | CST3 | JUNB | SERINC5 | FA2H |
| NASP | CKAP2L | SOX2 | SSTR2 | DKK3 | CDH13 | MARCKSL1 |
| RAD51AP1 | CKAP2 | TFDP2 | SOX9 | ID4 | CXADR | LIMS2 |
| GMNN | AURKB | CORO1C | RND3 | ADCYAP1R1 | LHFPL3 | PHLDB1 |
| WDR76 | BUB1 | EIF4B | EDNRB | GLUL | ARL4A | RAB33A |
| SLBP | KIF11 | FBLIM1 | GABRB1 | PFKFB3 | SHD | OPCML |
| CCNE2 | ANP32E | SPDYE7P | PLTP | CPE | RPL31 | SHISA4 |
| UBR7 | TUBB4B | TCF4 | JUNB | ZFP36L1 | GAP43 | TMEFF2 |
| POLD3 | GTSE1 | ORC6 | DKK3 | JUN | IFITM10 | NME1 |
| MSH2 | KIF20B | SPDYE1 | ID4 | SLC1A3 | SIRT2 | NXPH1 |
| ATAD2 | HJURP | NCRUPAR | ADCYAP1R1 | CDC42EP4 | OMG | GRIA4 |
| RAD51 | HJURP | BAZ2B | GLUL | NTRK2 | RGMB | SGK1 |
| RRM2 | CDCA3 | NELL2 | EPAS1 | CBS | HIPK2 | ZDHHC9 |
| CDC45 | HN1 | OPHN1 | PFKFB3 | DOK5 | APOD | CSPG4 |
| CDC6 | CDC20 | SPHKAP | ANLN | FOS | NPPA | LRRN1 |
| EXO1 | TTK | RAB42 | HEPN1 | TRIL | EEF1B2 | BIN1 |
| TIPIN | CDC25C | LOH12CR2 | CPE | SLC1A2 | RPS17L | EBP |
| DSCC1 | KIF2C | ASCL1 | RASL10A | ATP13A4 | FXYD6 | CNP |
| BLM | RANGAP1 | BOC | SEMA6A | ID1 | MYT1 | |
| CASP8AP2 | NCAPD2 | ZBTB8A | ZFP36L1 | TPCN1 | RGR | |
| USP1 | DLGAP5 | ZNF793 | HEY1 | FOSB | OLIG2 | |
| CLSPN | CDCA2 | TOX3 | PRLHR | LIX1 | ZCCHC24 | |
| POLA1 | CDCA8 | EGFR | TACR1 | IL33 | MTSS1 | |
| CHAF1B | ECT2 | PGM5P2 | JUN | TIMP3 | GNB2L1 | |
| BRIP1 | KIF23 | EEF1A1 | GADD45B | NHSL1 | C17orf76-AS1 | |
| E2F8 | HMMR | MALAT1 | SLC1A3 | ZFP36L2 | ACTG1 | |
| | AURKA | TATDN3 | CDC42EP4 | DTNA | EPN2 | |
| | PSRC1 | CCL5 | MMD2 | ARHGEF26 | PGRMC1 | |
| | ANLN | EVI2A | CPNE5 | TBC1D10A | TMSB10 | |
| | LBR | LYZ | CPVL | LHFP | NAP1L1 | |
| | CKAP5 | POU5F1 | RHOB | NOG | EEF2 | |
| | CENPE | FBXO27 | NTRK2 | LCAT | MIAT | |
| | CTCF | CAMK2N1 | CBS | LRIG1 | CDHR1 | |
| | NEK2 | NEK5 | DOK5 | GATSL3 | TRAF4 | |
| | G2E3 | PABPC1 | TOB2 | ACSL6 | TMEM97 | |
| | GAS2L3 | AFMID | FOS | HEPACAM | NACA | |
| | CBX5 | QPCTL | TRIL | SCG3 | RPSAP58 | |
| | CENPA | MBOAT1 | NFKBIA | RFX4 | SCD | |
| | | HAPLN1 | SLC1A2 | NDRG2 | TNK2 | |
| | | LOC90834 | MTHFD2 | HSPB8 | RTKN | |
| | | LRTOMT | IER2 | ATF3 | UQCRB | |
| | | GATM-AS1 | EFEMP1 | PON2 | FA2H | |
| | | AZGP1 | ATP13A4 | ZFP36 | MIF | |
| | | RAMP2-AS1 | KCNIP2 | PER1 | TUBB3 | |
| | | SPDYE5 | ID1 | BTG2 | COX7C | |
| | | TNFAIP8L1 | TPCN1 | NRP1 | AMOTL2 | |
| | | | LRRC8A | PRRT2 | THY1 | |
| | | | MT2A | F3 | NPM1 | |
| | | | FOSB | | MARCKSL1 | |
| | | | L1CAM | | LIMS2 | |
| | | | LIX1 | | PHLDB1 | |
| | | | HLA-E | | RAB33A | |
| | | | PEA15 | | GRIA2 | |
| | | | MT1X | | OPCML | |
| | | | IL33 | | SHISA4 | |
| | | | LPL | | TMEFF2 | |
| | | | IGFBP7 | | ACAT2 | |
| | | | C1orf61 | | HIP1 | |
| | | | FXYD7 | | NME1 | |
| | | | TIMP3 | | NXPH1 | |
| | | | RASSF4 | | FDPS | |
| | | | HNMT | | MAP1A | |
| | | | JUND | | DLL1 | |
| | | | NHSL1 | | TAGLN3 | |
| | | | ZFP36L2 | | PID1 | |
| | | | SRPX | | KLRC2 | |
| | | | DTNA | | AFAP1L2 | |
| | | | ARHGEF26 | | LDHB | |
| | | | SPON1 | | TUBB4A | |
| | | | TBC1D10A | | ASIC1 | |
| | | | DGKG | | TM7SF2 | |
| | | | LHFP | | GRIA4 | |
| | | | FTH1 | | SGK1 | |
| | | | NOG | | P2RX7 | |
| | | | LCAT | | WSCD1 | |
| | | | LRIG1 | | ATP5E | |
| | | | GATSL3 | | ZDHHC9 | |
| | | | EGLN3 | | MAML2 | |
| | | | ACSL6 | | UGT8 | |
| | | | HEPACAM | | C2orf27A | |
| | | | ST6GAL2 | | VIPR2 | |
| | | | KIF21A | | DHCR24 | |
| | | | SCG3 | | NME2 | |
| | | | METTL7A | | TCF12 | |
| | | | CHST9 | | MEST | |
| | | | RFX4 | | CSPG4 | |
| | | | P2RY1 | | GAS5 | |
| | | | ZFAND5 | | MAP2 | |
| | | | TSPAN12 | | LRRN1 | |
| | | | SLC39A11 | | GRIK2 | |
| | | | NDRG2 | | FABP7 | |
| | | | HSPB8 | | EIF3E | |
| | | | IL11RA | | RPL13A | |
| | | | SERPINA3 | | ZEB2 | |
| | | | LYPD1 | | EIF3L | |
| | | | KCNH7 | | BIN1 | |
| | | | ATF3 | | FGFBP3 | |
| | | | TMEM151B | | RAB2A | |
| | | | PSAP | | SNX1 | |
| | | | HIF1A | | KCNIP3 | |
| | | | PON2 | | EBP | |
| | | | HIF3A | | CRB1 | |
| | | | MAFB | | RPS10-NUDT3 | |
| | | | SCG2 | | GPR37L1 | |
| | | | GRIA1 | | CNP | |
| | | | ZFP36 | | DHCR7 | |
| | | | GRAMD3 | | MICAL1 | |
| | | | PER1 | | TUBB | |
| | | | TNS1 | | FAU | |
| | | | BTG2 | | TMSB4X | |
| | | | CASQ1 | | PHACTR3 | |
| | | | GPR75 | | | |
| | | | TSC22D4 | | | |
| | | | NRP1 | | | |
| | | | DNASE2 | | | |
| | | | DAND5 | | | |
| | | | SF3A1 | | | |
| | | | PRRT2 | | | |
| | | | DNAJB1 | | | |
| | | | F3 | | | |

### Reporters

The organoid and/or glioma cells may comprise one or more reporters (e.g., reporter genes and expression products thereof). The reporters may be used to monitor the formation of glioma in the composition and/or characterize various types of cells or tissues. The glioma cells may be modified to express one or more reporter genes prior to being added to the organoid. Alternatively or additionally, cells in the organoid may express one or more reporter genes. In some cases, glioma cells and cells in the organoid express different reporter genes.

In general, a reporter gene may be a gene that is not endogenous or native to the host cells and that encodes a protein that can be readily assayed. Reporter genes may be fluorescent, luminescent, enzymatic and resistance genes. Examples of reporter genes include detectable marker genes, e.g., genes encoding fluorescent proteins such as green fluorescent protein (GFP), cyan fluorescent protein (CFP), yellow fluorescent protein (YFP), and autofluorescent proteins including blue fluorescent protein (BFP), glutathione-S-transferase (GST), horseradish peroxidase (HRP), chloramphenicol acetyltransferase (CAT) beta-galactosidase, beta-glucuronidase, luciferase, HcRed, DsRed, cell surface markers, antibiotic resistance genes such as neo, and the like.

The reporters may also be selectable marker genes, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene.

### Genetic Variations

The organoid and/or glioma cells may comprise one or more genetic variations. In some cases, one or more variations may be introduced in the organoid, and the variation(s) may have an effect on the glioma cells. In certain cases, one or more variations may be introduced in the glioma cells, and the variation(s) may have an effect on the organoid. For example, modifications to cellular adhesion molecules (e.g., extracellular receptors, synaptic proteins, etc.) may be introduced on the organoid cells and/or the glioma cells to inhibit intercellular interaction/communication.

In some cases, the one or more variations includes those related to the development and progression of glioma. Examples of genetic variations include those described in Wang L E et al., Polymorphisms of DNA repair genes and risk of glioma, Cancer Res. 2004 Aug. 15; 64(16):5560-3; Liu Y et al., Genetic advances in glioma: susceptibility genes and networks, Curr Opin Genet Dev. 2010 June; 20(3):239-44; Schwartzbaum J A et al., Epidemiology and molecular pathology of glioma, Nat Clin Pract Neurol. 2006 September; 2(9):494-503; quiz 1 p following 516; Zhang J et al., Whole-genome sequencing identifies genetic alterations in pediatric low-grade gliomas. Nat Genet. 2013 June; 45(6):602-12. doi: 10.1038/ng.2611.

### Methods of Tumor Modeling

The present disclosure further provides methods of modeling a tumor in vitro. In some embodiments, the methods can be used for modeling glioma, the method comprising implanting one or more glioma cells to a brain organoid. For example, the methods may comprise implanting patient-derived glioma cells to a dorsal forebrain organoid. In some cases, the dorsal forebrain organoid has a core comprising less than 25% apoptotic or hypoxic cells.

### Generation of Organoids

The organoids may be derived from one or more progenitor cells. The progenitors may be cultured in one or a series of media, allowing the progenitors to differentiate into desired types of cells.

### Stem Cells

The progenitor cells may be stem cells. The organoids may be generated by differentiating one or more types of stem cells into desired cells that naturally present in a brain. The stem cells may be capable, under appropriate conditions, of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Examples of stem cells include those listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

The stem cells may be from or derived from embryonic tissues (e.g., fetal or pre-fetal tissues), or adult tissues. The stem cells may be isolated from or derived from cells isolated from tissues such as skin, fat tissue (e.g. adipose tissue), muscle tissue, heart or cardiac tissue, umbilical cord blood, placenta, bone marrow, or chondral.

A mixture of cells from a suitable source of endothelial, muscle, and/or neural stem cells can be harvested from a mammalian donor by methods known in the art. A suitable source is the hematopoietic microenvironment. For example, circulating peripheral blood, mobilized (e.g., recruited), may be removed from a subject. Alternatively, bone marrow may be obtained from a mammal, such as a human patient, undergoing an autologous transplant. In some embodiments, stem cells can be obtained from the subject's adipose tissue, for example using the CELUTION SYSTEM from Cytori, as disclosed in U.S. Pat. Nos. 7,390,484 and 7,429,488 which are incorporated herein in their entirety by reference.

In some embodiments, thawing, maintenance, and passaging of human pluripotent stem cells are performed by the methods described in Arlotta, P. et al. Long-term culture and electrophysiological characterization of human brain organoids, Protocol Exchange dx.doi.org/10.1038/protex.2017.049 (2017).

Stem cells may be propagated and continuously in culture, using culture conditions that promote proliferation without promoting differentiation. Exemplary serum-containing stem cell medium is made with 80% DMEM (such as Knock-Out DMEM, Gibco), 20% of either defined fetal bovine serum (FBS, Hyclone) or serum replacement (WO 98/30679), 1% nonessential amino acids, 1 mM L-glutamine, and 0.1 mM β-mercaptoethanol. Just before use, human bFGF may be added to 4 ng/mL.

Stem cells may be cultured on a layer of feeder cells, typically fibroblasts derived from embryonic or fetal tissue. SCs may be maintained in an undifferentiated state even without feeder cells. The environment for feeder-free cultures may include a suitable culture substrate, particularly an extracellular matrix such as MATRIGEL® or laminin. Enzymatic digestion may be halted before cells become completely dispersed (about 5 min with collagenase IV). Clumps of about 10 to 2,000 cells may be then plated directly onto the substrate without further dispersal.

Feeder-free cultures may be supported by a nutrient medium containing factors that support proliferation of the cells without differentiation. Such factors may be introduced into the medium by culturing the medium with cells secreting such factors, such as irradiated (about 4,000 rad) primary mouse embryonic fibroblasts, telomerized mouse fibroblasts, or fibroblast-like cells derived from pPS cells. Medium may be conditioned by plating the feeders at a density of about 5-6×10⁴ cm⁻² in a serum free medium such as KO DMEM supplemented with 20% serum replacement and 4 ng/mL bFGF. Medium that has been conditioned for 1-2 days may be supplemented with further bFGF, and used to support pluripotent SC culture for 1-2 days.

### Embryonic Stem Cells

The stem cells may be embryonic stem (ES) cells. ES cells may be undifferentiated when they have not committed to a specific differentiation lineage. Such cells may display morphological characteristics that distinguish them from differentiated cells of embryo or adult origin. Undifferentiated ES cells may appear in the two dimensions of a microscopic view in colonies of cells with high nuclear/cytoplasmic ratios and prominent nucleoli. Undifferentiated ES cells may express genes that can be used as markers to detect the presence of undifferentiated cells, and whose polypeptide products may be used as markers for negative selection.

The ES cells may be human ES cells, which express cell surface markers that characterize undifferentiated nonhuman primate ES and human EC cells, including stage-specific embryonic antigen (SSEA)-3, SSEA-4, TRA-1-60, TRA-1-81, and alkaline phosphatase. Methods for proliferating hES cells in the undifferentiated form are described in WO 99/20741, WO 01/51616, and WO 03/020920.

### Reprogramed or Induced Stem Cells

In some embodiments, the stem cells may be reprogrammed stem cells, such as stem cells derived from somatic or differentiated cells. In such an embodiment, the reprogramed stem cells can be for example, but not limited to, neoplastic cells, tumor cells and cancer cells or alternatively induced reprogrammed cells such as induced pluripotent stem cells or iPS cells.

In some cases, the stem cells may be induced pluripotent stem cells. In some examples, the organoid is derived from PGP1 (Personal Genome Project 1) hiPSC (human induced pluripotent stem cells); HUES66 hESC (human embryonic stem cells); 11a hiPSC; GM08330 hiPSC; or Mito 210 hiPSC.

### Exemplary Methods for Generating Dorsal Forebrain Organoids

The dorsal forebrain organoids herein may be generated from different HuESCs and iPSCs each having consistent cell types and cell proportions. In some examples, a dorsal forebrain organoid may be generated by culturing an aggregate of pluripotent stem cells (e.g., iPS cells) in suspension in the presence of a Wnt signal inhibitor and a TGFβ signal inhibitor, culturing the dorsal forebrain progenitor marker-positive aggregate in a spinner flask at about 20% oxygen (e.g., atmospheric oxygen levels) and 5% CO2.

Any suitable method may be used to culture an aggregate of pluripotent stem cells in suspension. In some embodiments, stem cells are dissociated into single cells and then cultured in low attachment tissue culture plates, spinner flasks, or aggrewell plates. In some embodiments, the cells are disassociated in the presence of a ROCK inhibitor (e.g., Y-27632). In some embodiments, the dissociated cells are cultured in cortical differentiation medium. In some embodiments, the cortical differentiation medium (CDM) is serum free. In some embodiments, the cortical differentiation medium is further supplemented with a ROCK inhibitor (e.g., Y-27632). In some embodiments, the CDM is supplemented with a ROCK inhibitor for about the first 6 days of culture.

The Wnt signal inhibitor and the TGFβ signal inhibitor are not limited and may be any suitable inhibitors known in the art. In some embodiments, the TGFβ signal inhibitor is SB431542 (e.g., SB431542 to a final concentration of about 5 μM). In some embodiments, the Wnt signal inhibitor IWR1 (e.g., IWR1 to a final concentration of 3 μM).

In some embodiments, the cells are cultured for about 16-20 day (e.g., 18 days) in 96 v-well low attachment plates (e.g., prime surface 96V plates), thereby forming aggregates. In some embodiments, the cells are cultured at a concentration of about 8000-10,000 (e.g., 9000) cells per well in a volume of about 100 μl. In some embodiments, the cells are cultured at 37° C. and 5% CO2. In some embodiments, the cells are cultured without shaking. During culturing, the CDM media should be changed/replenished as needed. In some embodiments, the CDM media is changed about every three days.

In some embodiments, after culturing for about 16-20 day (e.g., 18 days), the cell aggregates are transferred to 100 mm ultra-low attachment tissue culture plates and further cultured with CDM media. In some embodiments, the CDM media comprises N-2 supplement. During culturing, the CDM media should be changed/replenished as needed. In some embodiments, the CDM media is changed about every three days. In some embodiments, the CDM media does not comprise a Wnt signal inhibitor or a TGFβ signal inhibitor. In some embodiments, about 40-60 (e.g., about 48) aggregates are transferred into a 100 mm ultra-low attachment tissue culture plate with about 15 ml of media. In some embodiments, the aggregates are cultured in the tissue culture plates at 37° C. and 5% CO2 for about 15-20 days (e.g., 17 days). In some embodiments, the aggregates are cultured with shaking (e.g., on an orbital shaker). In some aspects, the rotation rate of the orbital shaker is about 5 RPM, 10 RPM, 15 RPM, 20 RPM, 25 RPM, 30 RPM, 35 RPM, 40 RPM, 45 RPM, 50 RPM, 55 RPM, 60 RPM, 65 RPM, 70 RPM, 75 RPM, 80 RPM, 85 RPM, 90 RPM, 95 RPM, 100 RPM, 105 RPM, 110 RPM, 115 RPM, 120 RPM, 125 RPM, 130 RPM, 135 RPM, 140 RPM, 145 RPM, or 150 RPM. In some aspects, the rotation rate of the orbital shaker is a rate that allows sufficient oxygen diffusion in the medium and at the same time preserves the integrity of the aggregates. In some aspects, the rotation rate of the orbital shaker that allows enough oxygen diffusion in the medium and at the same time preserves the integrity of the aggregates is about 60-80 rpm, preferably about 70 rpm.

In some embodiments, after culturing for about 30-40 days (e.g., 35 days), the cell aggregates may be transferred to a spinner flask. In some embodiments, culturing cell aggregates for about 30-40 days as detailed herein produces DFOs as described herein (e.g., DFOs cultured for about a month). In some embodiments, about 90-100 cell aggregates (now organoids) are added to a 125 ml spinner flask containing about 100 ml of CDM media. In some embodiments, the CDM media comprises serum (e.g., fetal bovine serum). In some embodiments, the CDM media comprises heparin. In some embodiments, the CDM media comprises N-2 supplement. In some embodiments, the CDM media comprises heparin.

In some embodiments, the organoids are cultured in a spinner flask at 37° C. and 5% CO2 with stirring. In some embodiments, the stirring speed is about 30 RPM, 35 RPM, 40 RPM, 45 RPM, 50 RPM, 51 RPM, 52 RPM, 53 RPM, 54 RPM, 55 RPM, 56 RPM, 57 RPM, 58 RPM, 59 RPM, 60 RPM, 65 RPM, 70 RPM, 75 RPM, or 80 RPM. In some aspects, the stirring is at a speed that allows sufficient oxygen diffusion in the medium and at the same time preserves the integrity of the organoids. In some aspects, the stirring speed that allows enough oxygen diffusion in the medium and at the same time preserves the integrity of the organoids is about 50-60 rpm, preferably about 56 rpm. In some embodiments, the organoids are cultured for about 30-40 days (e.g., 35 days) with media change/replenishment as needed (see, e.g., “detailed protocol”). In some embodiments, the CDM media is changed about every 7 days.

In some embodiments, after about 30-40 days (e.g., 35 days) of culturing in spinner flasks, the formulation of the CDM media is changed. In some embodiments, the new CDM media comprises serum (e.g., fetal bovine serum). In some embodiments, the new CDM media comprises heparin. In some embodiments, the new CDM media comprises N-2 supplement. In some embodiments, the new CDM media comprises heparin. In some embodiments, the new CDM media comprises B-27 supplement. In some embodiments, the organoids are cultured in the spinner flask at 37° C. and 5% CO2 with stirring. The stirring speed is not limited and may be any suitable stirring speed described herein. In some embodiments, the stirring speed is about 56 RPM.

In some embodiments, the organoids may be cultured in a spinner flask for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 months or more.

In some embodiments, the methods described herein produce multiple organoids having highly similar cell types and cell proportions. In some embodiments, the methods described herein produce a plurality of organoids having a mutual information (MI) score of less than 0.1, less than 0.09, less than 0.08, less than 0.07, less than 0.06, less than 0.05, less than 0.049, less than 0.045, less than 0.042, or less than 0.03. In some embodiments, the MI score for organoids produced after culture for about 3 months is less than about 0.06, 0.05, or 0.049. In some embodiments, the MI score for organoids produced after culture for about 6 months is less than 0.1, less than 0.09, or less than 0.089. In some embodiments, the MI scores have Z-scores (divergence of the MI score for individual organoids from the mean MI score expected at random) of less than 80, less than 70, less than 60, less than 50, less than 50, less than 40, or less than 30. In some embodiments, the z-score for organoids produced after culture for about 3 months is less than 45.0, less than 40.0, or less than 38.0. In some embodiments, the z-score for organoids produced after culture for about 6 months is less than 85.0, less than 80.0, or less than 75.7. In some embodiments, the organoids produced by the methods disclosed herein have an intraclass correlation (ICC) of more than 0.65, more than 0.68, more than 0.70, more than 0.75, more than 0.80, more than 0.85, or more than 0.90. In some embodiments, the ICC for organoids cultured for 3 months by the methods described herein are 0.80 or more (e.g., 0.85 or more). In some embodiments, the ICC for organoids cultured for 6 months or more by the methods described herein are 0.60 or more (e.g., 0.68 or more).

### Implantation of Glioma Cells

One or more glioma cells may be implanted into the organoid. After implantation, the glioma cells may form tumor-like cells or tissues in the organoid. In some cases, the organoid provides a microenvironment for the tumor cells to grow and progress, thus mimicking the initiation, formation, and/or progression of tumors in a subject, such as a patient.

In some cases, the glioma cells may be implanted onto the surface of an organoid. In such an embodiment, patient-derived glioma cells growing in Neurosphere culture (DMEM F12 media+Neurobasal media+EGF/FGF growth factors, grown in low-attachment culture-ware) may first be dispersed into single cells using a variety of enzymatic methods (Accutase, Trypsin/TrypLE). Organoids and dispersed glioma cells may then be co-cultured (in CDM media, minus matrigel) in low-binding dishes or plates with constant rotation and periodic mechanical agitation. In some aspects, a proper cell/organoid ratio (e.g., 150,000 glioma cells per organoid, in one well of a 24-well plate) and culture conditions (e.g., 70 rpm rotation, with manual trituration every 15 minutes, for 2 hours) are maintained such that the glioma cells remain dispersed while allowing for a fraction to spontaneously adhere to the surface of the organoid within 6-72 hours. After sufficient co-culture time, organoids may be transferred back to normal growth conditions (CDM media in spinning bioreactor or low-attachment petri dish on a shaker) and the glioma cells may infiltrate/colonize the organoid. The cultures may be analyzed at arbitrary post-implantation time points (e.g., 2 weeks) for imaging, sequencing, etc. In some cases, different patient-derived glioma lines may have different properties (size, morphology, growth dynamics, fragility, propensity to grow as single cells or spheres, etc.) that require fine-tuning of the above details.

### Introducing Genetic Variations

One or more genetic variations may be introduced to the organoid and/or the glioma cells. The genetic variations may be introduced to the organoid before or during generation of the organoid. Alternatively or additionally, the genetic variations may be introduced to the organoid after implantation of the glioma cells. In some cases, the genetic variations may be introduced to the glioma cells before implantation. Alternatively or additionally, the genetic variations may be introduced to the glioma cells after implantation.

Various methods may be used for introducing the genetic variations. The genetic variations may be introduced by RNA targeting agents, such as RNAi, miRNA, or ribozyme. In some cases, the genetic variations may be introduced gene editing systems or components thereof. Examples of gene editing systems include CRISPR-Cas systems, zinc finger nuclease systems, TALEN systems, and meganuclease systems.

Examples of methods for introducing genetic variations using CRISPR-Cas systems include those described in Shalem O, et al., High-throughput functional genomics using CRISPR-Cas9, Nat Rev Genet. 2015 May; 16(5):299-311; Sanjana N E, et al., Genome-scale CRISPR pooled screens, Anal Biochem. 2017 Sep. 1; 532:95-99; Miles L A, et al., Design, execution, and analysis of pooled in vitro CRISPR/Cas9 screens, FEBS J. 2016 September; 283(17):3170-80; Ford K, et al., Functional Genomics via CRISPR-Cas, J Mol Biol. 2019 Jan. 4; 431(1):48-65.

The CRISPR-Cas systems may include those with additional functional domains and proteins, such as base editors (e.g., those described in Cox D B T, et al., RNA editing with CRISPR-Cas13, Science. 2017 Nov. 24; 358(6366):1019-1027; Abudayyeh O O, et al., A cytosine deaminase for programmable single-base RNA editing, Science 26 Jul. 2019: Vol. 365, Issue 6451, pp. 382-386; Gaudelli N M et al., Programmable base editing of A•T to G•C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19; 533(7603):420-4; Jordan L. Doman et al., Evaluation and minimization of Cas9-independent off-target DNA editing by cytosine base editors, Nat Biotechnol (2020)), prime editing systems (e.g., those described in Anzalone A V et al., Search-and-replace genome editing without double-strand breaks or donor DNA, Nature. 2019 Oct. 21. doi: 10.1038/s41586-019-1711-4), CAST systems (e.g., those described in Strecker J et al., RNA-guided DNA insertion with CRISPR-associated transposases. Science. 2019 Jul. 5; 365(6448):48-53; Klompe S E, et al., Transposon-encoded CRISPR-Cas systems direct RNA-guided DNA integration. Nature. 2019 July; 571(7764):219-225).

Examples of methods for introducing genetic variations using other gene editing systems and RNAi include those described in Peng Y, et al., Making designer mutants in model organisms. Development. 2014 November; 141(21):4042-54; Carroll D, et al., Genome engineering with targetable nucleases, Annu Rev Biochem. 2014; 83:409-39; Govindan G, et al., Programmable Site-Specific Nucleases for Targeted Genome Engineering in Higher Eukaryotes. J Cell Physiol. 2016 November; 231(11):2380-92; Gaj T, et al., ZFN, TALEN, and CRISPR/Cas-based methods for genome engineering, Trends Biotechnol. 2013 July; 31(7):397-405.

Additional examples of the methods include those described in Harris A L, et al., Patient-derived tumor xenograft models for melanoma drug discovery. Expert Opin Drug Discov. 2016 September; 11(9):895-906; Izumchenko E, et al., Patient-derived xenografts as tools in pharmaceutical development. Clin Pharmacol Ther. 2016 June; 99(6):612-21.

### EXEMPLARY APPLICATIONS

The compositions and systems herein may be used for various applications. In some embodiments, the compositions and system herein provide tumor models for studying the biology and underlying mechanisms of tumorigenesis and growth. For example, the growth rates, transcriptional states, cellular lineages and hierarchies, cell morphologies, tumor-organoid microenvironmental interactions, invasive potential of brain tumor, e.g., glioma, cells, intercellular communication, and/or intercellular connectivity of the brain tumor, e.g., glioma, cells may be tested on the compositions and systems.

### Methods of Identifying Genes and variations

In some embodiments, the compositions and systems may be used to identify genes and variations thereof related to tumor (e.g., glioma) initiation, formation and/or progression. For example, one or more genetic variations may be introduced to the organoid and/or the glioma cells, the growth rates, transcriptional states, cellular lineages and hierarchies, cell morphologies, glioma-organoid microenvironmental interactions, invasive potential of glioma cells, intercellular communication, and/or intercellular connectivity of the glioma cells may be tested. The results may then be compared to a control, e.g., a counterpart composition or system in which no such genetic variation is introduced. Role of the variations and modified genes may be then determined based on the comparison.

In certain embodiments, genes are screened by perturbation of target genes within the neuronal cells, tumor cells, or other types of cells in the composition or system. Methods and tools for genome-scale screening of perturbations include perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; and Adamson et al., “A Multiplexed Single-Cell CRISPR Screening Platform Enables Systematic Dissection of the Unfolded Protein Response” 2016, Cell 167, 1867-1882; Joung J et al, Genome-scale CRISPR-Cas9 knockout and transcriptional activation screening. Nat Protoc. 2017 April; 12(4):828-863; Aregger M et al., Pooled Lentiviral CRISPR-Cas9 Screens for Functional Genomics in Mammalian Cells. Methods Mol Biol. 2019; 1869:169-188). Examples of such methods also include those for introducing genetic variations described herein.

In certain embodiments, signature genes may be perturbed in single cells and gene expression analyzed. Not being bound by a theory, networks of genes that are disrupted due to perturbation of a signature gene may be determined. Understanding the network of genes effected by a perturbation may allow for a gene to be linked to a specific pathway that may be targeted to modulate the signature and treat a tumor. Thus, in certain embodiments, perturb-seq is used to discover novel drug targets to allow treatment of the modeled tumor.

In some embodiments, the method comprises (1) introducing single-order or combinatorial perturbations to a population of cells, (2) measuring genomic, genetic, proteomic, epigenetic and/or phenotypic differences in single cells and/or (3) assigning a perturbation(s) to the single cells.

A perturbation may be linked to a phenotypic change, e.g., changes in gene or protein expression. In some embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures, when as to the cell population level, refer to genes that are differentially expressed in all or substantially all cells of the population (such as at least 80%, preferably at least 90%, such as at least 95% of the individual cells). This can allow one to define a particular subpopulation of cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

In further aspects, the invention relates to gene signatures, protein signature, and/or other genetic or epigenetic signature of particular astrocyte subpopulations, as defined herein elsewhere.

ScRNA-seq may be obtained from cells using standard techniques known in the art. Some exemplary scRNA-seq techniques are discussed elsewhere herein. As discussed elsewhere herein, a collection of mRNA levels for a single cell can be called an expression profile (or expression signature) and is often represented mathematically by a vector in gene expression space. See e.g. Wagner et al., 2016. Nat. Biotechnol; 34(111): 1145-1160. This is a vector space that has a dimension corresponding to each gene, with the value of the ith coordinate of an expression profile vector representing the number of copies of mRNA for the ith gene. Note that real cells only occupy an integer lattice in gene expression space (because the number of copies of mRNA is an integer), but it is assumed herein that cells can move continuously through a real-valued G dimensional vector space.

In certain embodiments, the measuring of phenotypic differences and assigning a perturbation to a single cell is determined by performing single cell RNA sequencing (RNA-seq). In preferred embodiments, the single cell RNA-seq is performed by any method as described herein (e.g., Drop-seq, InDrop, 10×genomics). In certain embodiments, unique barcodes are used to perform Perturb-seq. In certain embodiments, a guide RNA is detected by RNA-seq using a transcript expressed from a vector encoding the guide RNA. The transcript may include a unique barcode specific to the guide RNA. Not being bound by a theory, a guide RNA and guide RNA barcode is expressed from the same vector and the barcode may be detected by RNA-seq.

Not being bound by a theory, detection of a guide RNA barcode is more reliable than detecting a guide RNA sequence, reduces the chance of false guide RNA assignment and reduces the sequencing cost associated with executing these screens. Thus, a perturbation may be assigned to a single cell by detection of a guide RNA barcode in the cell. In certain embodiments, a cell barcode is added to the RNA in single cells, such that the RNA may be assigned to a single cell. Generating cell barcodes is described herein for single cell sequencing methods. In certain embodiments, a Unique Molecular Identifier (UMI) is added to each individual transcript and protein capture oligonucleotide. Not being bound by a theory, the UMI allows for determining the capture rate of measured signals, or preferably the binding events or the number of transcripts captured. Not being bound by a theory, the data is more significant if the signal observed is derived from more than one protein binding event or transcript. In preferred embodiments, Perturb-seq is performed using a guide RNA barcode expressed as a polyadenylated transcript, a cell barcode, and a UMI.

In some embodiment, the method further comprises performing epigenetic screening. In some examples, epigenetic screening is performed by applying CRISPRa/i/x technology (see, e.g., Konermann et al. “Genome-scale transcriptional activation by an engineered CRISPR-Cas9 complex” Nature. 2014 Dec. 10. doi: 10.1038/nature14136; Qi, L. S., et al. (2013). “Repurposing CRISPR as an RNA-guided platform for sequence-specific control of gene expression”. Cell. 152 (5): 1173-83; Gilbert, L. A., et al., (2013). “CRISPR-mediated modular RNA-guided regulation of transcription in eukaryotes”. Cell. 154 (2): 442-51; Komor et al., 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimizing deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035).

Numerous genetic variants associated with disease phenotypes are found to be in non-coding regions of the genome, and frequently coincide with transcription factor (TF) binding sites and non-coding RNA genes. Not being bound by a theory, CRISPRa/i/x approaches may be used to achieve a more thorough and precise understanding of the implication of epigenetic regulation. In one embodiment, a CRISPR system may be used to activate gene transcription. A nuclease-dead RNA-guided DNA binding domain, e.g., dCas, tethered to transcriptional repressor domains that promote epigenetic silencing (e.g., KRAB) may be used for “CRISPRi” that represses transcription. To use dCas as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In one embodiment, CRISPR-Cas systems may be used to perturb protein-coding genes or non-protein-coding DNA. CRISPR-Cas systems may be used to knockout protein-coding genes by frameshifts, point mutations, inserts, or deletions. An extensive toolbox may be used for efficient and specific CRISPR-Cas systems mediated knockout as described herein, including a double-nicking CRISPR to efficiently modify both alleles of a target gene or multiple target loci and a smaller Cas protein for delivery on smaller vectors (Ran, F. A., et al., In vivo genome editing using Staphylococcus aureus Cas9. Nature. 520, 186-191 (2015)). A genome-wide sgRNA mouse library (˜10 sgRNAs/gene) may also be used in a mouse that expresses a Cas protein (see, e.g., WO2014204727A1).

In one embodiment, perturbation is by deletion of regulatory elements. Non-coding elements may be targeted by using pairs of guide RNAs to delete regions of a defined size, and by tiling deletions covering sets of regions in pools.

In one embodiment, perturbation of genes is by RNAi. The RNAi may be shRNA's targeting genes. The shRNA's may be delivered by any methods known in the art. In one embodiment, the shRNA's may be delivered by a viral vector. The viral vector may be a lentivirus, adenovirus, or adeno associated virus (AAV).

In certain embodiments, whole genome screens can be used for understanding the phenotypic readout of perturbing potential target genes. In preferred embodiments, perturbations target expressed genes as defined by a gene signature using a focused sgRNA library. Libraries may be focused on expressed genes in specific networks or pathways. In other preferred embodiments, regulatory drivers are perturbed. In certain embodiments, Applicants perform systematic perturbation of key genes in neuronal and glioma cells in a high-throughput fashion. Applicants can use gene expression profiling data to define the target of interest and perform follow-up single-cell and population RNA-seq analysis. Not being bound by a theory, this approach will accelerate the development of therapeutics for tumors and oncology disease as described herein.

In some embodiments, the methods may comprise identifying differentially expressed genes in the tumor cells before and after implantation into organoids; filtering out genes and/or coherent signatures that have relevant functional (e.g., Gene Ontology) annotations; and/or filtering out genes and/or signatures that are not expressed by a minimal subset of cells in the analogous patient tumor (e.g., <5%) or tumor type.

### Methods for Screening Therapeutic Agents

In some embodiments, the compositions and systems may be used to screen therapeutic agents for treating the tumor or related health problems. In general, the compositions or systems may be contacted with one or more candidate agents. The effects of the candidate agent(s) on the organoid and/or tumor may be assessed. The results may be used to identify the desired agent(s). For example, the methods may comprise contacting the composition or systems with one or more candidate agents; and testing effects of the one or more candidate agents on growth rates, transcriptional states, cellular lineages and hierarchies, cell morphologies, tumor-organoid microenvironmental interactions, invasive potential of tumor, e.g., glioma, cells, intercellular communication, and/or intercellular connectivity of the tumor, e.g., glioma cells.

Examples of agents that may be identified or screened using the methods include small molecules, nucleic acids, polypeptides, peptides, drugs, ions and salts thereof. An agent may be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties include unsubstituted or substituted alkyl, aromatic, or heterocyclyl moieties including macrolides, leptomycins and related natural products or analogues thereof. Compounds can be known to have a desired activity and/or property, or can be selected from a library of diverse compounds. The agents also include the gene editing systems or components thereof, e.g., CRISPR-Cas systems.

The methods may be used for determining the therapeutic effects of one or more agents (e.g., on glioma). The term “therapeutic effect” refers to some extent of relief of one or more of the symptoms of a disorder (e.g., a tumor) or its associated pathology. The methods may further be used to determine a therapeutically effective amount of an agent. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., EDO of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

In some embodiments, the methods comprise screening a library of compounds or biologic molecules (e.g., polynucleotides or nucleic acids). The library may be a library of polynucleotides, e.g., libraries of natural polypeptides in the form of bacterial, fungal, plant, and animal extracts or modified forms thereof. The natural and synthetically produced libraries are produced, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al, Proc. Natl. Acad. Sci. USA 91:11422, 1994; Zuckermann et al., J. Med. Chem. 37:2678, 1994; Cho et al, Science 261:1303, 1993; Carrell et al, Angew. Chem. Int. Ed. Engl. 33:2059, 1994; Carell et al, Angew. Chem. Int. Ed. Engl. 33:2061, 1994; and Gallop et al, J. Med. Chem. 37: 1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Libraries of compounds may be presented in solution, or on beads, chips, bacteria, spores, plasmids or on phage. Such compounds and molecules libraries may be used in the screening methods herein. For example, the methods may be used for screening alkylating agents (e.g., those described in Strobel H, et al., Temozolomide and Other Alkylating Agents in Glioblastoma Therapy. Biomedicines. 2019 Sep. 9; 7(3). pii: E69), pyrazolopyrimidines (e.g., those described in Valero T, et al., Pyrazolopyrimide library screening in glioma cells discovers highly potent antiproliferative leads that target the PI3K/mTOR pathway. Bioorg Med Chem. 2020 Jan. 1; 28(1):115215), serotonergic blockers, cholesterol-lowering agents (statins), antineoplastics, anti-infective, anti-inflammatories, and hormonal modulators (e.g., those described in Jiang P, et al., Novel anti-glioblastoma agents and therapeutic combinations identified from a collection of FDA approved drugs. J Transl Med. 2014 Jan. 17; 12:13).

In some cases, the contacting step refers to incubating the agent and composition/system together in vitro. The composition or system contacted with an agent can also be simultaneously or subsequently contacted with another agent. In some embodiments, the composition or system is contacted with at least two, at least three, at least four, at least five, at least six, at least seven, at least eight, at least nine, or at least ten agents.

In some embodiments, the screening assays appropriate to the cell type and agent and/or environmental factor will be used in the methods. For example, changes in cell morphology may be assayed by standard light, or electron microscopy. The effects of treatments by the agent potentially affecting the expression of one or more genes may be assayed by measuring the expression level of the genes. As another example, the effects of treatments or compounds which potentially alter the pH or levels of various ions within cells may be assayed using various dyes which change in color at determined pH values or in the presence of particular ions. The use of such dyes is well known in the art. For cells which have been transformed or transfected with a genetic marker, such as the β-galactosidase, alkaline phosphatase, or luciferase genes, the effects of treatments or compounds may be assessed by assays for expression of that marker. In particular, the marker may be chosen so as to cause spectrophotometrically assayable changes associated with its expression.

In some embodiments, cytotoxicity of the agents may be tested. Cytotoxicity can be determined by the effect on cell viability, morphology, and leakage of enzymes into the culture medium. In certain embodiments, toxicity may be assessed by observation of vital staining techniques, ELISA assays, immunohistochemistry, and the like or by analyzing the cellular content of the culture, e.g., by total cell counts, and differential cell counts or by metabolic markers such as MTT and XTT. In some embodiments, a colorimetric assay can be performed to quantitatively measure LDH released into the media from cells as a biomarker for cellular cytotoxicity and cytolysis (e.g. ThermoFisher Scientific cat. #88953). For these embodiments, culture mediums can be collected without disassociated the composition or system. These collections can occur at different timepoints and/or regular intervals (e.g. every 24 hours) to measure lactate dehydrogenase (LDH) released from the tissue as a result of gene perturbation.

In some embodiments, the disclosure provides a method for assessing the metabolism of a therapeutic agent by one or more types of cell in the composition or system. The method may comprise exposing the composition or system to a candidate agent, and determining the effect of the neuronal cells on the agent. For example, the effect may be measured by detecting, identifying, and/or quantifying metabolites of the agent.

The method may further comprise effects of the agent on expression and activity of genes or gene products. Detection of changes in expression of genes and/or gene products can be assayed by any method known in the art including immunohistochemistry, immunofluorescence, flow cytometry, polymerase chain reaction (PCR), quantitative PCR, real-time PCR, gene expression array, mRNA sequencing, high-throughput sequencing, Western blot, Northern blot, and ELISA.

### Additional Exemplary Methods of Characterization of Tumor and its Effects

The in vitro models described herein are suitable for array-based gene screening in combination with one or more of electrophysiological measurements, calcium imaging for activity, and fluorescent/bioluminescent imaging for phenotype.

Fluorescent and/or bioluminescent staining may be performed by methods known in the art. In some embodiments, cells are fixed (e.g. with paraformaldehyde or ethanol) and, if applicable, frozen to enable slicing with a cryostat (e.g. Leica CM1950). In some embodiments, sections of cell encapsulating hydrogels are cut at 10-30 μm thickness and washed with DPBS to remove freezing medium before immunostaining. In some embodiments, samples are blocked with a blocking reagent (e.g. serum) and then incubated with primary antibodies. In some embodiments, samples are mounted (e.g. using Prolong Diamond Antifade Mountant with DAPI (Thermo-Fisher Scientific)) and imaged using a fluorescent microscope (e.g. Zeiss AX10, Zeiss LSM710). Non-limiting examples of primary antibodies suitable for immunostaining include: mouse anti-Map2 (M4403, Sigma, 1:300-500); rabbit anti-Pax6 (901301, BioLegend, 1:300); chicken anti-GFAP (ab4674, Abcam, 1:500); mouse anti-S100β (ab11178, Abcam, 1:500); rabbit anti-Vimentin (5741, Cell Signaling, 1:100).

For electrophysiological measurements, whole cell voltage-clamp and current-clamp recordings may be performed. In some embodiments, the compositions and systems are infected with AAV U6-hSyn1-mCherry-KASH-hGH vectors encoding non-targeting sgRNA 6 days after forming the tissues to identify iN cells in 3D cultures. Recordings are performed in room temperature using K-Gluconate based intracellular solution (in mM: 131 K-Gluconate, 17.5 KCl, 9 NaCl, 10 HEPES, 1.1 EGTA, 1 MgCl2, 2 Mg-ATP and 0.2 Na-GTP) and artificial cerebrospinal fluid (in mM: 119 NaCl, 2.3 KCl, 1 NaH2PO4, 11 Glucose, 26.2 NaHCO3, 1.3 MgCl2, 2.5 CaCl2) as the external solution. Data is recorded using, for example pClamp 10 (Molecular Devices). Spontaneous synaptic currents are recorded with the voltage clamped at about −70 mV. In some embodiments, membrane capacitance and resistance are measured using a pClamp membrane test. In some embodiments, the resting membrane potential is recorded under a current clamp configuration. In some embodiments, current voltage relationships of the neurons are recorded under a current clamp configuration, where changes in voltage and subsequent action potentials are recorded after injecting hyperpolarizing and depolarizing currents (−200 pA to +200 pA, 50 pA steps). In some embodiments, recordings are performed using a patch pipette with a resistance ranging from 3-5 mΩ.

In some aspects, culture media can be collected without disassociation of the compositions and systems. These collections can occur at different timepoints and/or regular intervals (e.g. every 24 hours) to measure lactate dehydrogenase (LDH) released from the tissue as a result of gene perturbation. In some embodiments, a colorimetric assay can be performed to quantitatively measure LDH released into the media from cells as a biomarker for cellular cytotoxicity and cytolysis (e.g. ThermoFisher Scientific cat. #88953). The high-throughput array provides population level data relating to gene perturbations on neuronal and/or astrocytic cells in a disease context.

In some aspects, gRNA vectors for gene perturbations of neuronal and/or astrocytic cells are fluorescently labeled (e.g. mCherry-KASH under the control of the hSyn1 promoter) to independently label one or both cell types. After gene perturbations, the compositions and systems can be dissociated, and the cells can be sorted by flow cytometry cell sorting and placed into wells. In some embodiments, a fluorometric apoptosis assay is performed to detect caspases in microplates and determine a specific stage of apoptosis (e.g. Roche cat. #CASPASSY-RO). This approach provides cell-specific data in both array based and pooled screening of genes.

### EXAMPLES

### Example 1

Applicant developed novel glioma models based on the implantation of patient-derived glioma cells into human brain organoids. Primary glioma cells grown in human brain organoids may show a spectrum of cell states and phenotypes that are more faithful to human gliomas than currently existing in vitro glioma models. Moreover, Applicant carried out studies to demonstrate the utility of these glioma models for interrogating spatiotemporal mechanisms of disease progression and identifying therapeutic vulnerabilities in patients. The studies shown in the example are to develop and validate human brain organoid-based glioma models for studying human glioma behavior. Applicant developed methods to reproducibly implant a variety of patient-derived glioma cells (adult, pediatric, and IDH-mutant) into human brain organoids. Applicant used single cell genomics and 2D/3D imaging to compare molecular profiles (e.g., cell transcriptional states) and phenotypes (e.g., morphology and connectivity) between in vitro models and patient tumors, defining the scope of model validty. The studies shown in the example can be used to further interrogate the temporal dynamics underlying glioma progression and treatment evasion in human brain organoids. Applicant can use single cell genomics to assess and determine the transcriptional mechanisms of glioma progression. By leveraging real-time scRNA-seq sampling, Applicant can monitor how glioma cell states change during malignant progression, both normally and in response to selection (e.g., canonical molecular inhibitors). Predominant cell states can be correlated to cellular morphologies and environmental context (e.g., spatial localization in the organoid).

The studies described in the example are also to determine the effect of cellular perturbations on glioma growth and function in human brain organoids. In this aim, Applicant can selectively knock out subsets of genes in patient-derived glioma cells using CRISPR-based lentiviral constructs. The following parameters can be monitored for the engineered glioma lines growing in organoids: i) growth rate, ii) single cell transcriptional profiles, iii) tumor hierarchies, iv) cellular morphologies, and v) intercellular connectivity. These studies can highlight potential targeted therapeutic opportunities for treating human gliomas.

The studies shown in the example also demonstrate the capacity for intercellular communication within the model system. Applicant engineered glioma cells to express different fluorescent reporters and demonstrated that the reporters are transferred from glioma cells to the surrounding cells of the brain organoid parenchyma. This framework was used to identify genes that are differentially expressed between brain organoid cells that communicated with glioma cells (have the glioma reporter) and those that were not in communication (did not have the glioma reporter). These studies point towards mechanistic understanding of how glioma cells condition the surrounding microenvironment to promote tumor growth.

The overall goal of the studies is to develop and leverage novel, more faithful in vitro glioma models for interrogating spatiotemporal mechanisms of human glioma behavior. Gliomas, a class of molecularly diverse adult and pediatric primary brain tumors, have high mortality rates and remain incurable despite continued intense efforts on many fronts. Recent advances in glioma biology have highlighted the heterogeneity and inter-cellular communication within these tumors; it follows that appropriate models for studying glioma behavior and progression—and, in turn, therapeutic avenues—must adequately recapitulate these key features. Indeed, patient-derived tumor xenografts (PDXs) are attractive and widely-used in vivo model systems for studying gliomas, despite their limitations. The development of complementary (and currently non-existent) in vitro glioma models that better capture the molecular and phenotypic spectrum of the corresponding human tumor would enable reliable disease modeling and therapeutic testing at unprecedented scale and spatiotemporal resolution, potentially leading to much-needed breakthroughs for the field.

The compartmentalization and emergent phenotypes of human gliomas are determined, in large part, by cooperative interactions between the intrinsic features of malignant cells and the tumor microenvironment. In this regard, a limitation of current in vitro glioma models (e.g., gliomaspheres) is the lack of appropriate environmental cues, leading to a prohibitively reductionist or skewed representation of the disease. In recent years, human brain organoids have emerged as promising 3D, in vitro model systems for partially recreating the cellular composition and function of the human brain. In the context of this research, human brain organoids could represent a potential construct through which to provide 3D, human-specific environmental cues to patient-derived glioma cells, at once addressing a significant limitation of current in vitro glioma models.

The following parameters were monitored for the engineered glioma lines growing in organoids: i) growth rate, ii) single cell transcriptional profiles, iii) tumor hierarchies, iv) cellular morphologies, and v) intercellular connectivity. Brain organoid cells were also profiled with scRNA-seq to identify factors involved in glioma-neural communication. These studies highlighted potential targeted therapeutic opportunities for treating human gliomas.

Overall, highly faithful in vitro glioma models enable disease modeling and therapeutic testing at greater scale and resolution than is currently available. Compared to the low throughput of in vivo models, faithful in vitro glioma models can be leveraged to test hundreds of different drug targets or genetic modifications, with greater confidence that results would translate to a clinical setting. Moreover, rapid technological advances in molecular profiling and imaging allow for dissection of spatiotemporal mechanisms with unprecedented, multi-scale resolution.

Implantation of Patient-Derived Glioma Cells into Human Brain Organoids

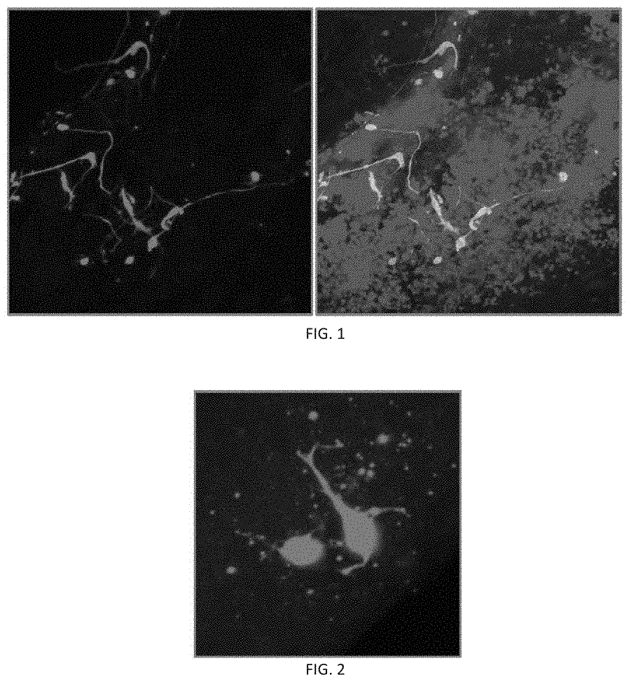

Applicant sought to develop reproducible methods to grow patient-derived glioma cell lines (in gliomasphere culture) within human brain organoids, to demonstrate the viability of our approach. FIG. 1 shows patient-derived glioblastoma cells stained with a live cell tracker dye (panel A) growing in human brain organoids stained with DAPI (panel B) after 3 days of co-culture. These results were reproduced across several different primary glioma lines, including those from adult, pediatric, and IDH-mutant gliomas.

Patient-Derived Glioma Cells Communicate via Projections and Vesicle-Like Structures

Applicant observed that many primary glioma cell lines demonstrated evidence of structural inter-connections and communicating structures. FIG. 1 and FIG. 2 show interconnecting tumor microtubes between individual tumor cells, in addition to extracellular vesicle-like structures that may also serve communicative functions. These results are in line with previous findings regarding communication between glioma cells, however the degree to which the structures are resolved in these images highlights an advantage of the in vitro glioma model system.

Patient-Derived Glioblastoma Cells Form Interconnected Networks in Human Brain Organoids

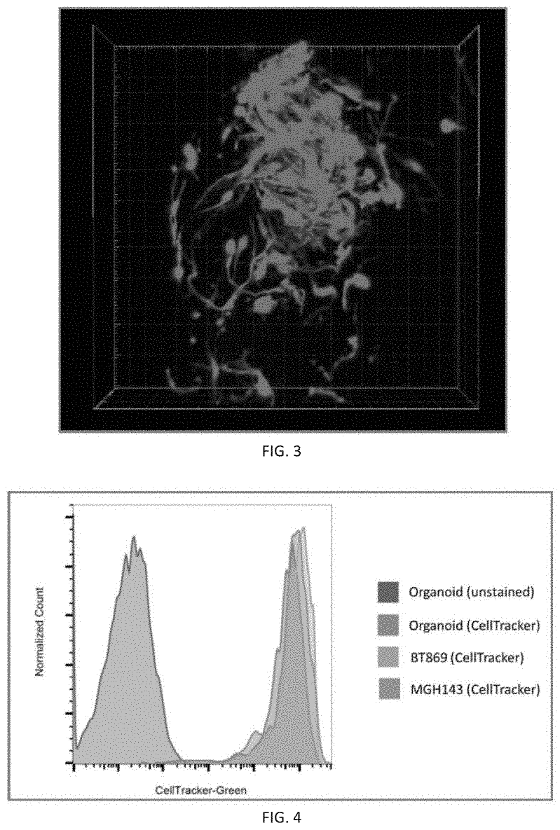

In addition to forming close range associations, glioma cells growing in human brain organoids also demonstrated larger-scale interconnectivity. FIG. 3 shows a 3D, interconnected glioblastoma cellular network growing in human brain organoids after 3 days of co-culture, imaged using confocal microscopy. These results highlighted the capacity of the in vitro glioma models that Applicant was developing to recapitulate emergent functions that are known to occur in patient tumors (and other in vivo models) and observed them at high spatiotemporal resolution. Applicant looked to identify molecular mediators of these microtube networks and demonstrate potential therapeutic opportunities to disrupt them.

Glioma-Brain Organoid Co-Cultures can be Dissociated with High Viability for scRNA-seq

Molecular profiling using scRNA-seq technologies were used in the studies. Applicant sought to demonstrate that Applicant could dissociate organoid-glioma co-cultures for scRNA-seq while retaining high viability of all cell types involved. FIG. 4 demonstrates that, under the same Papain-based dissociation conditions, both organoid and glioma cells showed high viability (using CellTracker dye as a viability stain). These results were independently confirmed using Trypan blue exclusion (not shown). These data show high cellular viability.

Patient-Derived Glioma Cells are Permissive of Lentiviral Transduction

FIG. 5 shows an image of primary DIPG gliomaspheres that were successfully infected with a GFP-expressing lentivirus. This result allows for simplified monitoring of glioma cells growing in human brain organoids via imaging. Furthermore, the GFP-tagged gliomaspheres allowed for simplified isolation of malignant cells from the brain organoids using dissociation procedures and flow cytometry. Applicant demonstrated here the potential to more generally infect primary gliomasphere lines with different types of lentiviral constructs.

Patient-Derived GFP-Tagged DIPG Cell Lines Show a Diversity of Cell Morphologies in Human Brain Organoids

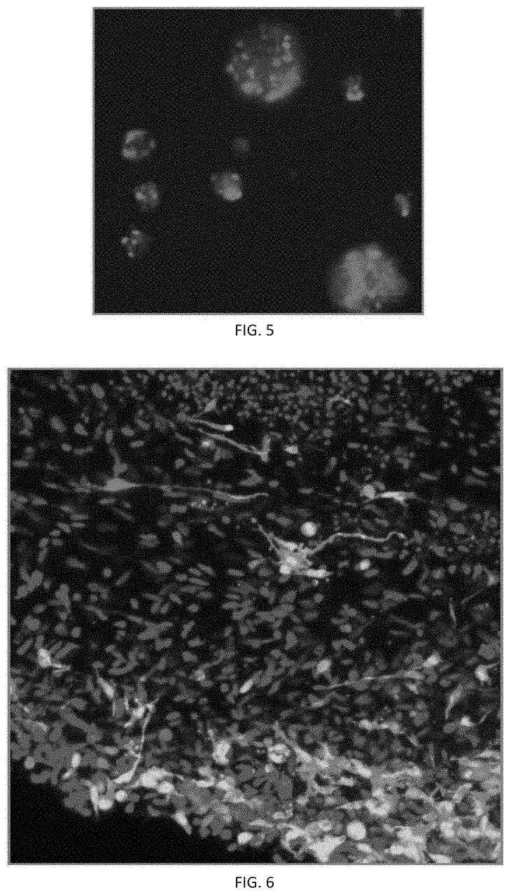

Patient-derived glioma lines growing in human brain organoids showed a spectrum of cell states and phenotypes that is representative of the human disease. Images, as shown in FIG. 6, of GFP-tagged DIPG cells infiltrating inside of a human brain organoid indicated an array of cellular morphologies that likely mapped to a corresponding spectrum of cell transcriptional states. There appeared to be an axis between relatively differentiated cell states (specialized structures and morphology) and undifferentiated states (unspecialized and anaplastic morphology) occurring in human DIPGs at the transcriptional level.

Development and Validation of Human Brain Organoid-Based Glioma Models for Studying Human Glioma Behavior

The ability to use brain organoids as a brain-mimicking environment for growing human gliomas in vitro had tremendous promise, however not much was known about the range of validity of this model system. Applicant sought to define this unknown by using molecular and phenotypic features of the human tumor as a grounding reference. This approach maximized the clinical relevance of the in vitro model system in disease modeling and drug testing use cases.

Research Design:

In the studies, Applicant demonstrated reproducible methods to grow and monitor patient-derived glioma lines (adult, pediatric, and IDH-mutant) in human brain organoids. Applicant continued to characterize newly-derived patient glioma lines that have a variety of genetic features and clinical characteristics (to the extent that these samples are available). Furthermore, Applicant carried out more comprehensive testing, using imaging and analytical flow cytometry, of the temporal development of each of these glioma lines in human brain organoids.

Glioma lines were tagged with GFP (starting with BT869, an H3K7M-mutant pediatric glioma line) and grown in organoids until a critical malignant/non-malignant cell percentage was reached (1-2%). The co-cultures subsequently were dissociated, and GFP-tagged glioma cells were isolated and sorted into 96-well plates for single-cell sequencing via the Smart-Seq2 protocol. Single cell transcriptomes were compared to those derived from the corresponding human tumor using standard computational methods. In addition to sequencing, Applicant also imaged 3D cellular morphologies and architectures of glioma cells in organoids and from analogous human tumor tissue. The resulting degree of concordance between transcriptional programs and cellular morphologies were used to define the scope of in vitro model validity.

The data demonstrated Applicant's capacity to reproducibly grow a variety of human gliomas within human brain organoids and dissociate these co-cultures for scRNA-seq, significantly de-risking the extent to which gliomas in brain organoids may recreate programs observed in human tumors.

Interrogation of the Intercellular Communication Between Glioma Cells and Parenchymal Cells in the Human Brain Organoid

Research Design:

Applicant previously demonstrated the capacity to grow patient derived glioma lines in brain organoids, monitor the glioma cells over time, dissociate the co-cultures and isolate malignant cells, and perform scRNA-seq. Applicant further demonstrated that brain organoid cells can be identified which have received reporters (e.g., GFP) from the adjacent glioma cells. Brain organoid cells that were GFP− and GFP+ were compared to identify factors that are involved with glioma conditioning of the surrounding microenvironment.

Interrogation of the Temporal Dynamics Underlying Glioma Progression and Treatment Evasion in Human Brain Organoids

Research Design:

Applicant previously demonstrated the capacity to grow patient-derived glioma lines in brain organoids, monitor the glioma cells over time, dissociated the co-cultures and isolated malignant cells, and performed scRNA-seq. Applicant can carry out real-time sampling of glioma-organoid co-cultures (from the same organoid batch) at time points determined to be functionally important (e.g., initial colonization, network formation, morphological alteration, unrestrained infiltration, etc.). Predominant cell states and hierarchical structures (e.g., developmental lineages) can be determined at each time point, providing a basis for the inference of major biologically-relevant temporal trajectories. Cell states can be correlated to cellular morphologies and environmental context using antibody or nucleic acid probes against cell-type markers in fixed tissue sections. Similar studies can be carried out on fully developed glioma-organoid models where addition of canonical molecular inhibitors (e.g., EGFR inhibitors for EGFR-amplified gliomas) marks the initial time point.

Determination of the Effect of Cellular Perturbations on Glioma Growth and Function in Human Brain Organoids

Applicant can show the capacity to successfully transduce patient-derived glioma lines using lentiviral vectors. Here Applicant can use this toolkit to transduce the same glioma lines with lentivirally-delivered CRISPR gene editing constructs. The panel of gene targets is selectively chosen by identifying major biological pathways and modules implicated in glioma pathogenesis. Based on glioma adaptation patterns under selection, Applicant additionally can consider gene targets for combinatorial use with molecular inhibitors.

For each engineered glioma line, Applicant can develop a human brain organoid co-culture model and characterize the following features (in comparison with the analogous un-perturbed model): a) growth rate, b) cell transcriptional states, c) cellular lineages/hierarchies, d) cell morphologies, and e) inter-cellular connectivity. In addition, a human brain organoid co-culture model can be used to characterize and identify factors involved in glioma-neural communication. The following technological modalities can be used: analytical flow cytometry (growth rate), scRNA-seq (cell states and hierarchies), confocal microscopy (cell morphologies), and tissue clearing and 3D imaging (inter-cellular connectivity).

### REFERENCES

Louis, D. N. et al. The 2016 World Health Organization Classification of Tumors of the Central Nervous System: a summary. Acta Neuropathol. 131, 803-820 (2016).
Tirosh, I. & Suvà, M. L. Dissecting human gliomas by single-cell RNA sequencing. Neuro. Oncol. 20, 37-43 (2017).
Osswald, M., Solecki, G., Wick, W. & Winkler, F. A malignant cellular network in gliomas: Potential clinical implications. Neuro. Oncol. 18, 479-485 (2016).
Charles, N. A., Holland, E. C., Gilbertson, R., Glass, R. & Kettenmann, H. The brain tumor microenvironment. Glia 59, 1169-1180 (2011).
Kelava, I. & Lancaster, M. A. Dishing out mini-brains: Current progress and future prospects in brain organoid research. Dev. Biol. 420, 199-209 (2016).
Wen, P. & Kesari, S. Malignant Gliomas in Adults—NEJM. Malig. gliomas adults 492-507 (2008). doi:10.1056/NEJMc086380
Filbin, M. G. & Suvà, M. L. Gliomas Genomics and Epigenomics: Arriving at the Start and Knowing It for the First Time. Annu. Rev. Pathol. Mech. Dis. 11, 497-521 (2016).
Venkatesh, H. S. et al. Neuronal activity promotes glioma growth through neuroligin-3 secretion. Cell 161, 803-816 (2015).
Osswald, M. et al. Brain tumour cells interconnect to a functional and resistant network. Nature 528, 93-98 (2015).
Anoop P. Patel, Itay Tirosh, John J. Trombetta, Alex K. Shalek, Shawn M. Gillespie, Hiroaki Wakimoto, Daniel P. Cahill, Brian V. Nahed, William T. Curry, Robert L. Martuza, David N. Louis, Orit Rozenblatt-Rosen, M. Single-cell RNA-seq highlights intratumoral heterogeneity in primary glioblastoma. Science (80-.). 346, 1396-1402 (2014).
Tirosh, I. et al. Single-cell RNA-seq supports a developmental hierarchy in human oligodendroglioma. Nature 539, 309-313 (2016).
Venteicher, A. S. et al. Decoupling genetics, lineages, and microenvironment in IDH-mutant gliomas by single-cell RNA-seq. Science (80-.). 355, eaai8478 (2017).
Filbin, M. G. et al. Developmental and oncogenic programs in H3K27M gliomas dissected by single-cell RNA-seq. Science (80-.). 360, 331-335 (2018).
Stiles, C. D. & Rowitch, D. H. Glioma Stem Cells: A Midterm Exam. Neuron 58, 832-846 (2008).
Weil, S. et al. Tumor microtubes convey resistance to surgical lesions and chemotherapy in gliomas. Neuro. Oncol. 19, 1316-1326 (2017).
Qin, E. Y. et al. Neural Precursor-Derived Pleiotrophin Mediates Subventricular Zone Invasion by Glioma. Cell 170, 845-859.e19 (2017).
Nakada, M., Hayashi, Y. & Hamada, J. Role of Eph/ephrin tyrosine kinase in malignant glioma. 13, 1163-1170 (2011).
Campbell, T. N. & Robbins, S. M. The Eph receptor/ephrin system: An emerging player in the invasion game. Curr. Issues Mol. Biol. 10, 61-66 (2008).
Katt, M. E., Placone, A. L., Wong, A. D., Xu, Z. S. & Searson, P. C. In Vitro Tumor Models: Advantages, Disadvantages, Variables, and Selecting the Right Platform. Front. Bioeng. Biotechnol. 4, (2016).
Huszthy, P. C. et al. In vivo models of primary brain tumors: Pitfalls and Perspectives. Neuro. Oncol. 14, 979-993 (2012).
Laks, D. et al. Neurosphere Formation Is an Independent Predictor of Clinical Outcome in Malignant Glioma. Stem Cells 980-987 (2009). doi:10.1002/6
Lenting, K., Verhaak, R., ter Laan, M., Wesseling, P. & Leenders, W. Glioma: experimental models and reality. Acta Neuropathol. 133, 263-282 (2017).
Lancaster, M. A. et al. Cerebral organoids model human brain development and microcephaly. Nature 501, 373-9 (2013).
Quadrato, G. et al. Cell diversity and network dynamics in photosensitive human brain organoids. Nature 545, 48-53 (2017).
Birey, F. et al. Assembly of functionally integrated human forebrain spheroids. Nature (2017). doi:10.1038/nature22330
Di Lullo, E. & Kriegstein, A. R. The use of brain organoids to investigate neural development and disease. Nat. Rev. Neurosci. 18, 573-584 (2017).
Quadrato, G., Brown, J. & Arlotta, P. The promises and challenges of human brain organoids as models of neuropsychiatric disease. Nat. Med. 22, 1220-1228 (2016).
Tanay, A. & Regev, A. Scaling single-cell genomics from phenomenology to mechanism. Nature 541, 331-338 (2017).
Richardson, D. S. & Lichtman, J. W. Clarifying Tissue Clearing. Cell 162, 246-257 (2015).

### Results

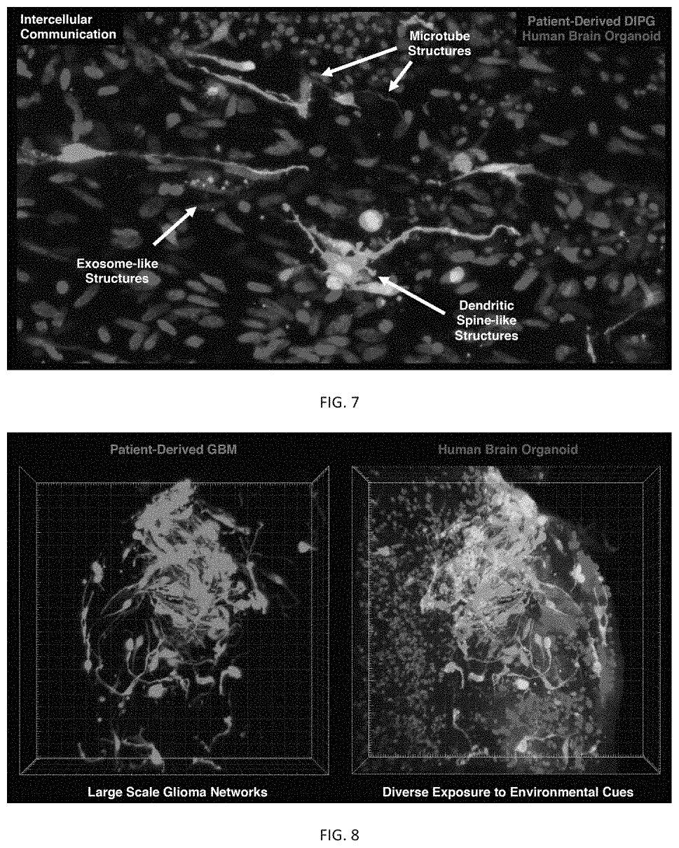

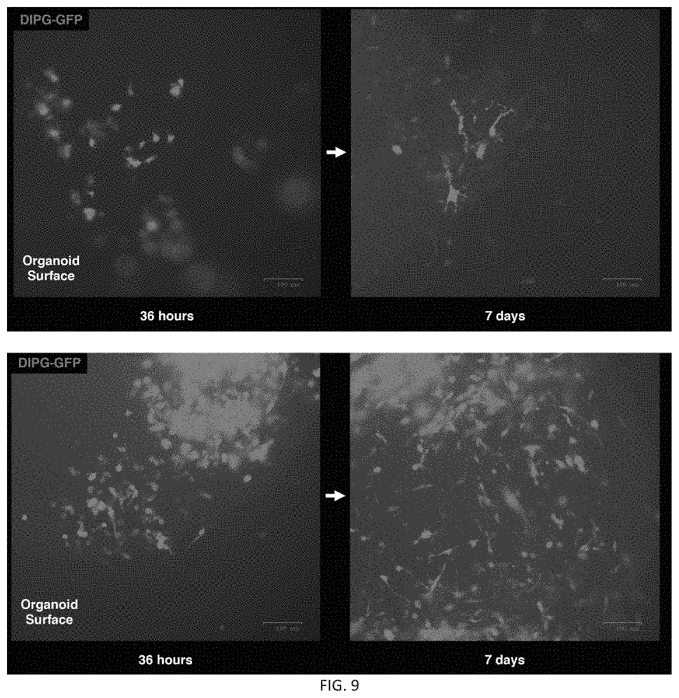

FIG. 7 shows glioma cells in a brain organoid. FIG. 8 shows diverse exposure to environmental cues. FIG. 9 shows temporal dynamics of glioma growth in brain organoids suggested strong environmental influence.

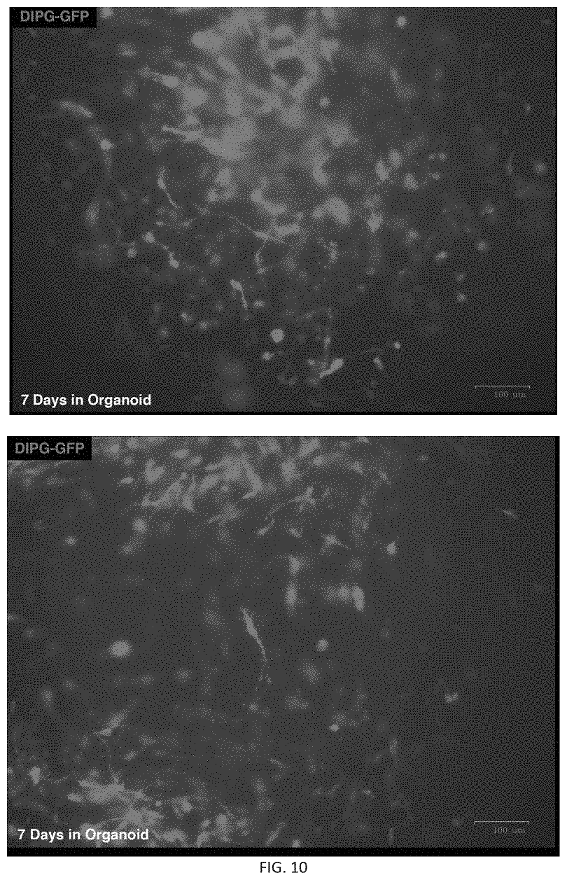

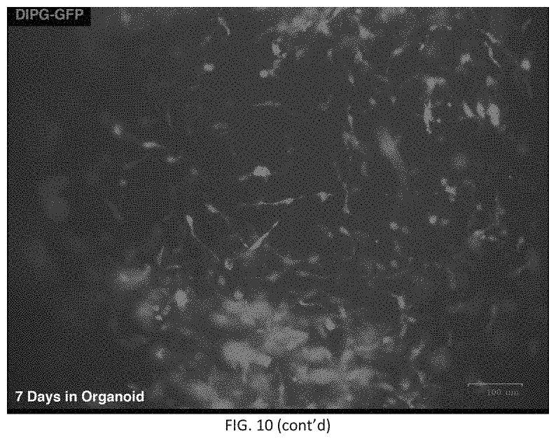

FIG. 10 shows patient-derived glioma cells exhibited striking morphological heterogeneity in human brain organoids. Glioma-brain organoid models recreated defining features of patient-specific disease (intercellular communication and cellular heterogeneity) in an in vitro setting.

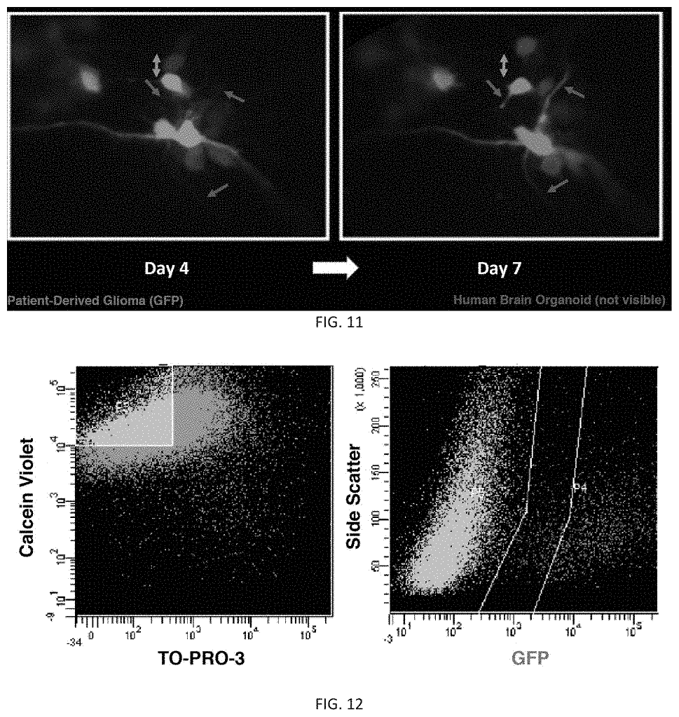

FIG. 11 shows transplant of an IDH1-R132H oligodendroglioma directly from a patient into a human brain organoid.

FIG. 12 shows healthy, GFP-tagged glioma cells are readily isolated from dissociated glioma-brain organoid co-cultures.

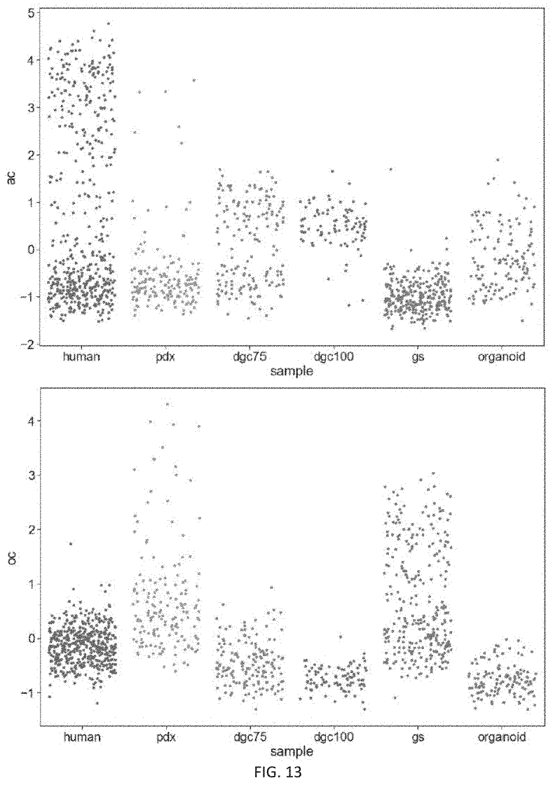

FIG. 13 shows the DIPG astrocyte-like signature.

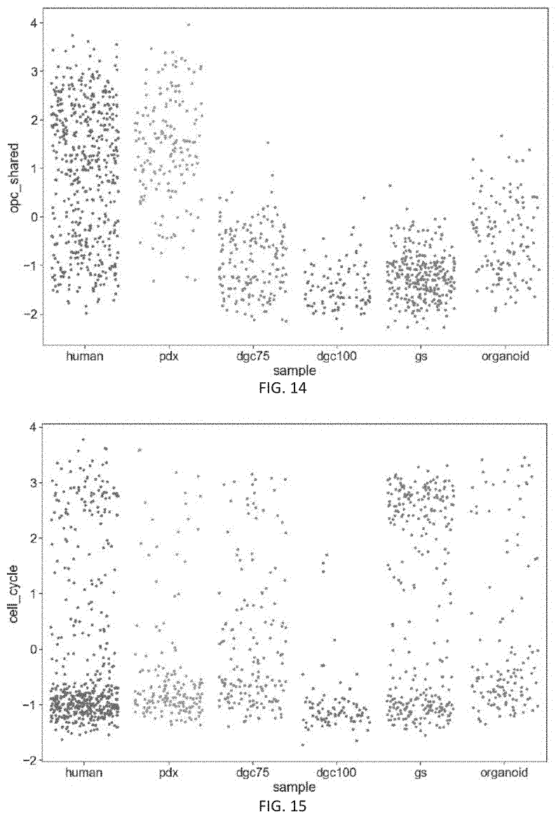

FIG. 14 shows the DIPG oligodendrocyte progenitor cell-like (shared) signature.

FIG. 15 shows the DIPG cell cycle signature.

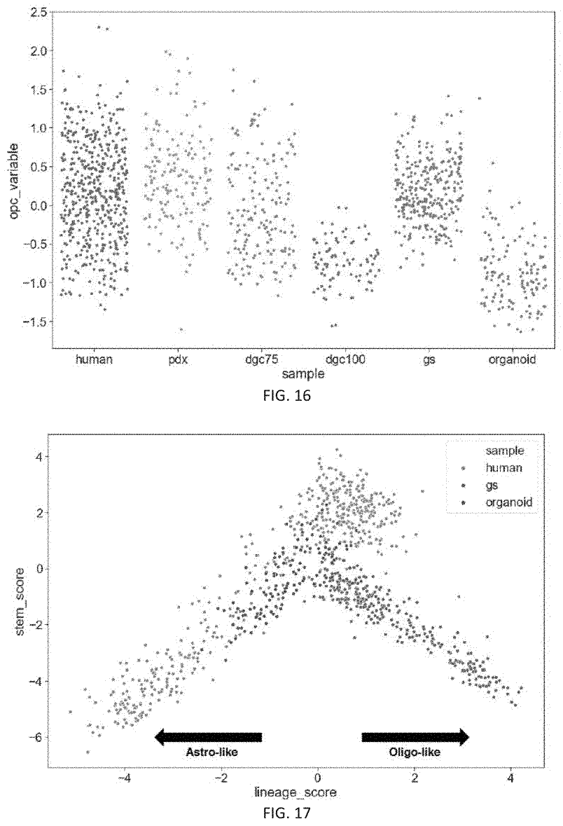

FIG. 16 shows the DIPG oligodendrocyte progenitor cell-like (variable) signature.

FIG. 17 shows the brain organoid microenvironment induced an OPC/OC-like to AC-like shift in patient-derived DIPG cells.

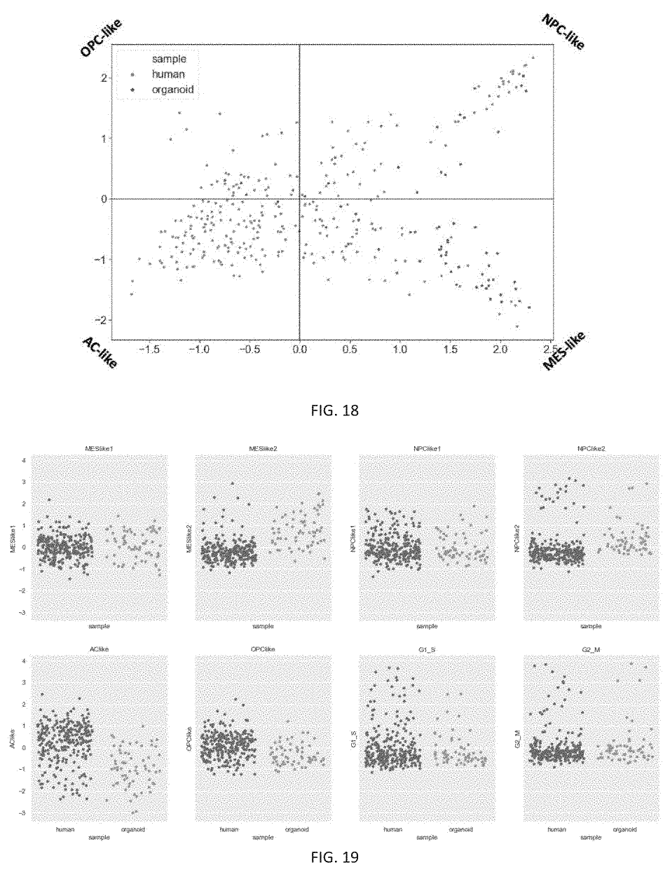

FIG. 18 shows cellular states represented in human gbm (mgh143) cells and an analogous human brain organoid model. Data Processing Steps included:1) Qualifying control (unique genes and housekeeping gene expression); 2) Calculating gene signature scores (shown individually on FIG. 19); 3) Classifying cells to a state based on maximum gene signature score (collapse NPC ½ and MES ½ states); 4) Constructing ‘cell-state’ plot (with each quadrant containing all cells mapped to a specific state from step 3). For this experiment, 270 human glioma cells and 66 cells from the glioma implanted organoid were included.

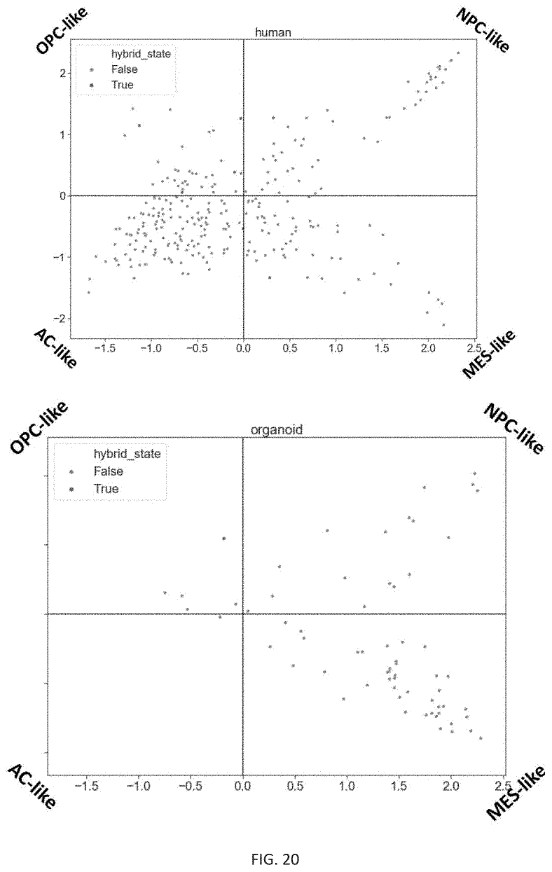

FIG. 20 shows hybrid states represented in human gbm (mgh143) cells and an analogous human brain organoid model.

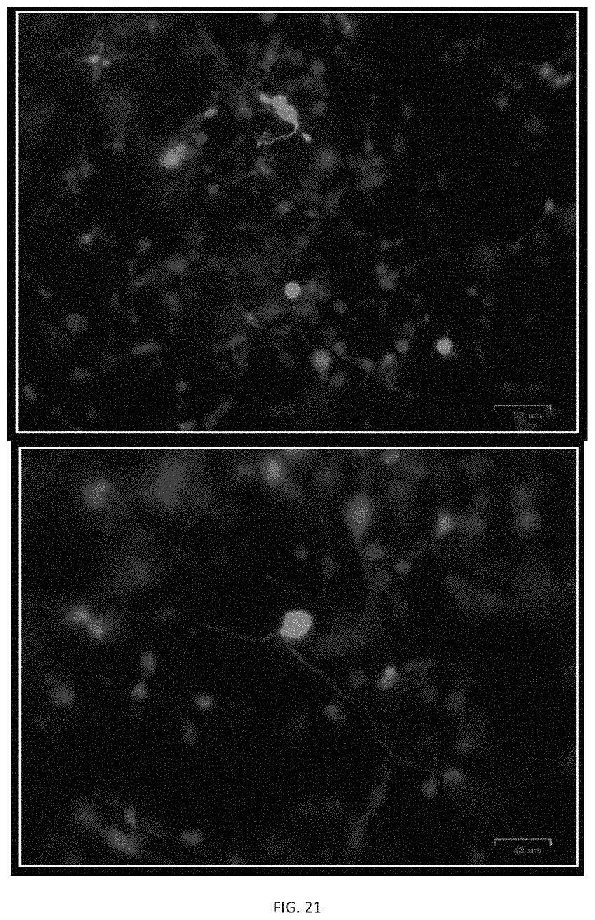

FIG. 21 shows correlating scRNA-seq results with matched imaging readouts.

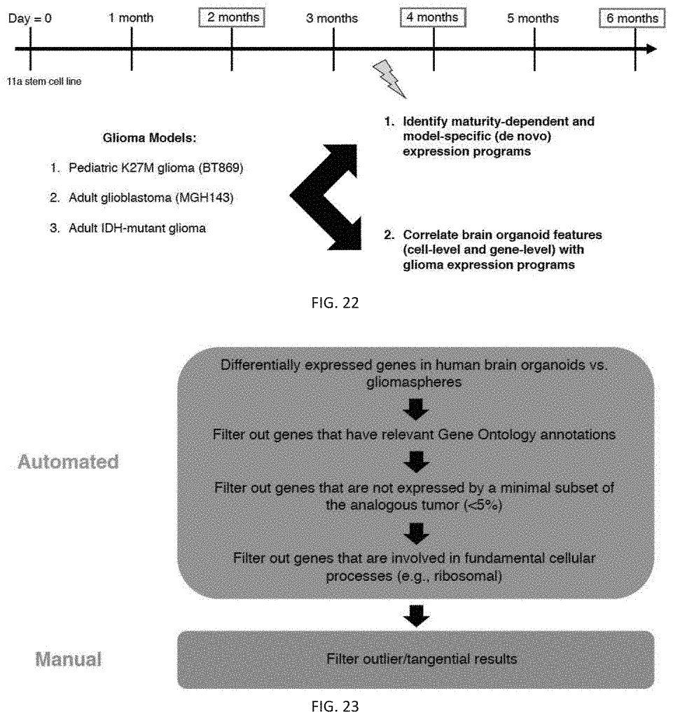

FIG. 22 shows an exemplary method for generating a glioma model and related organoid maturity and glioma model dependent cellular programs.

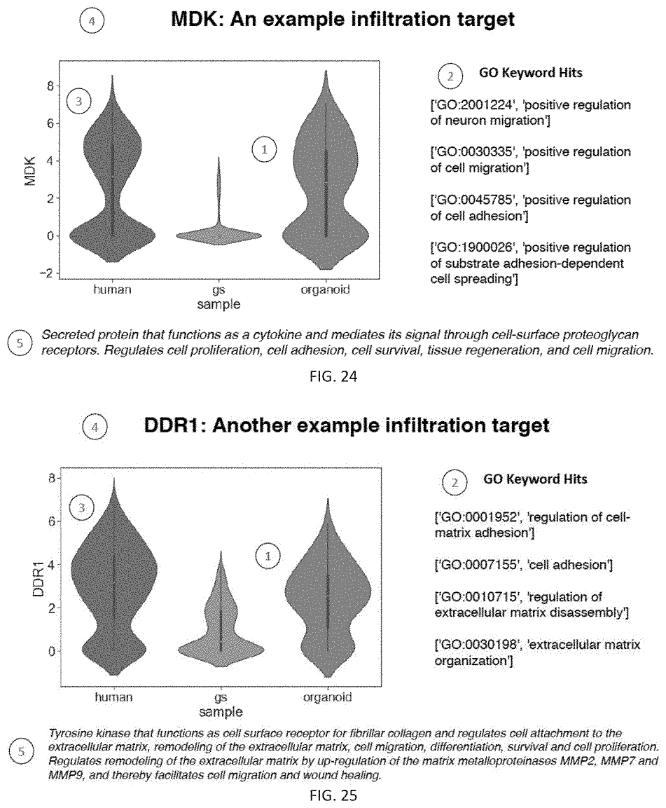

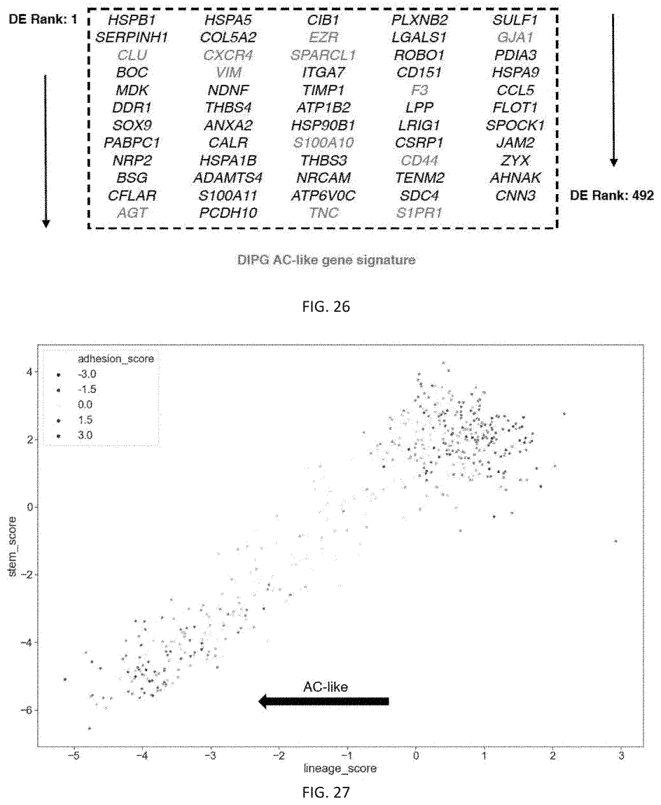

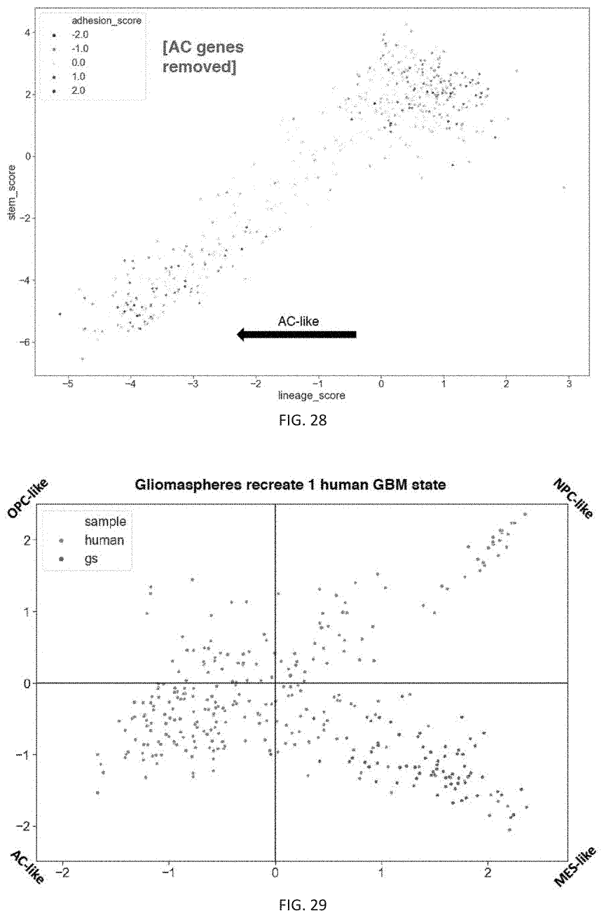

FIG. 23 shows an exemplary method for the identification of candidate targets for inhibiting glioma infiltration. FIG. 24 shows an example of infiltration target (MDK). FIG. 25 shows another example of infiltration target (DDR1). FIG. 26 shows candidate DIPG infiltration targets (adhesion molecules). Adhesion molecules were upregulated in an organoid model coordinately mapped to the AC-state of the human tumor (BCH869) (FIG. 27). FIG. 28 shows the result of FIG. 27 with AC gene removed.

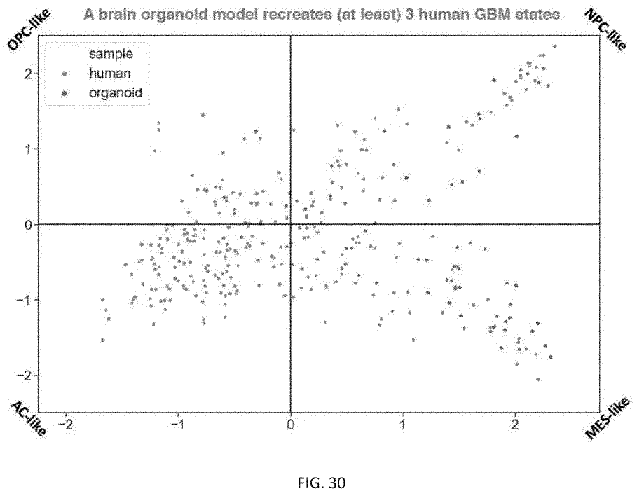

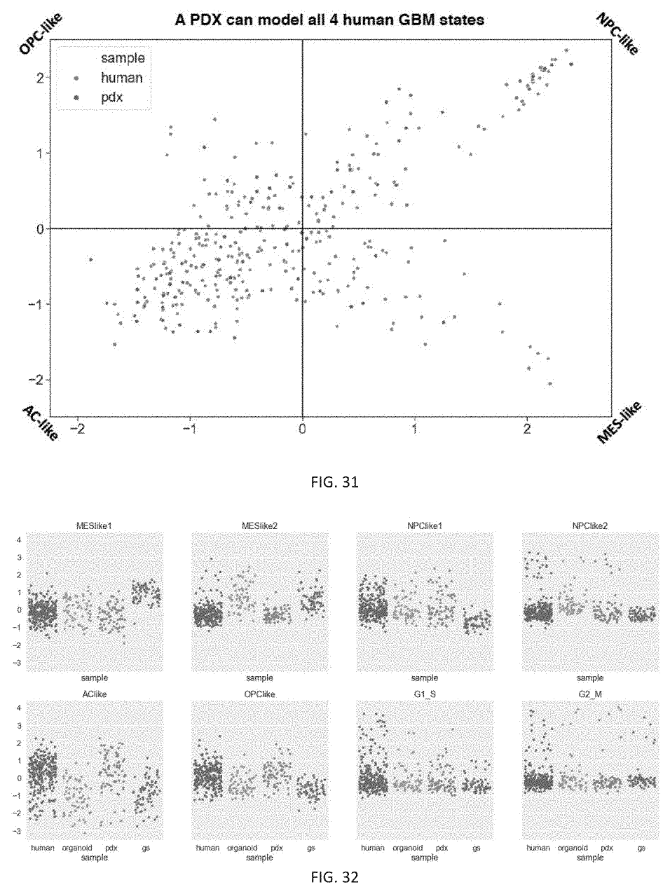

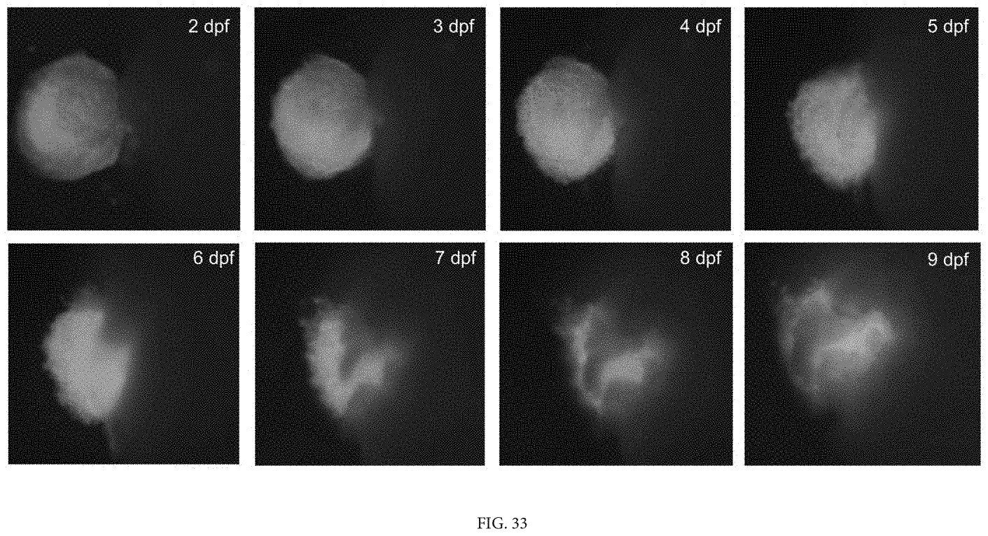

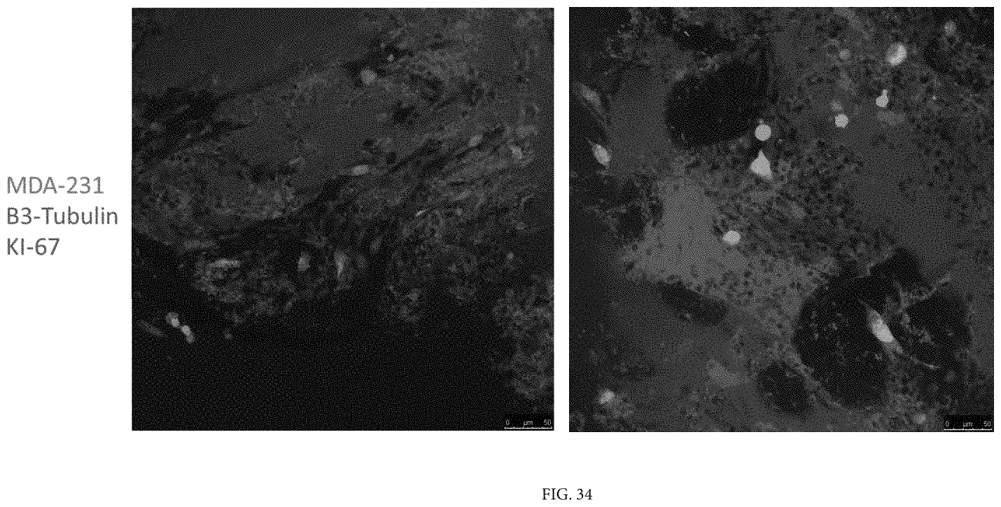

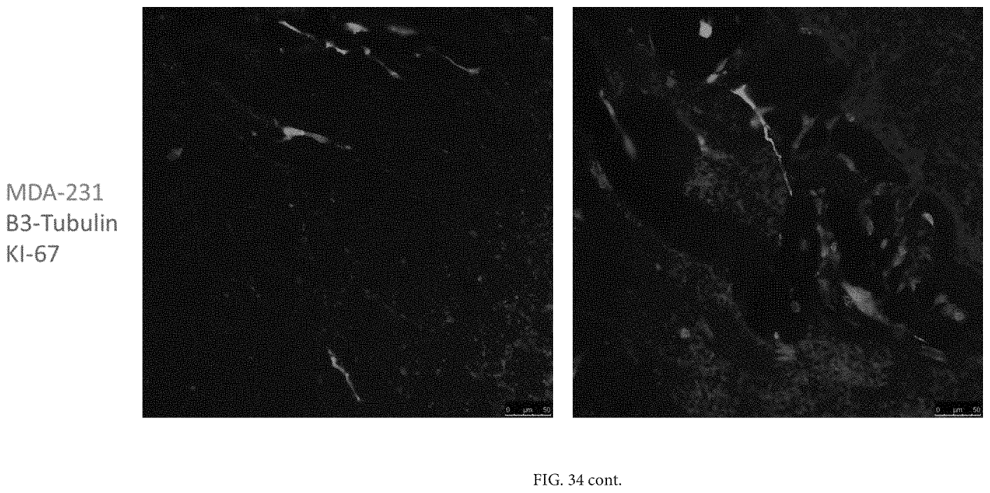

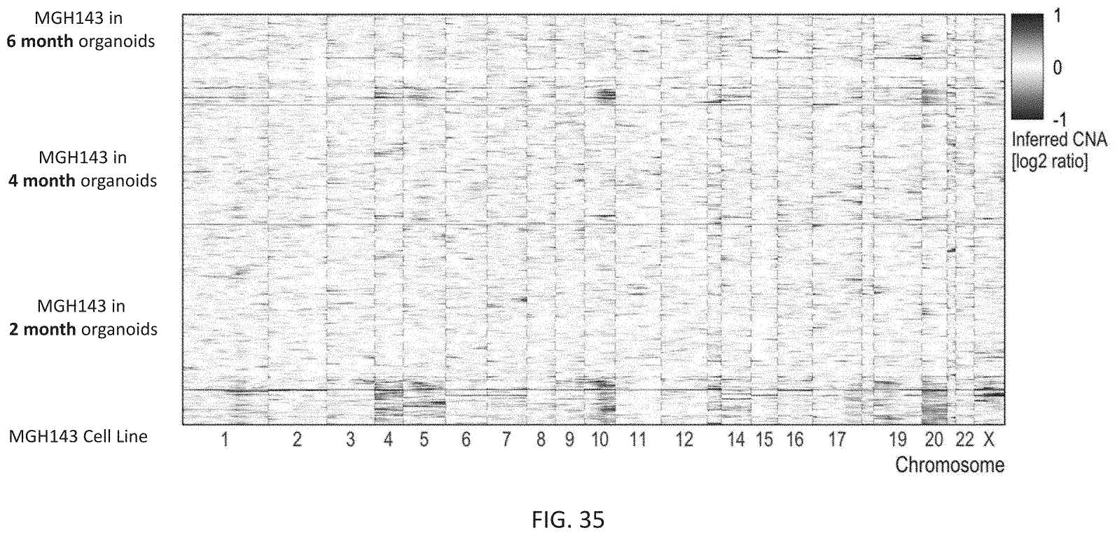

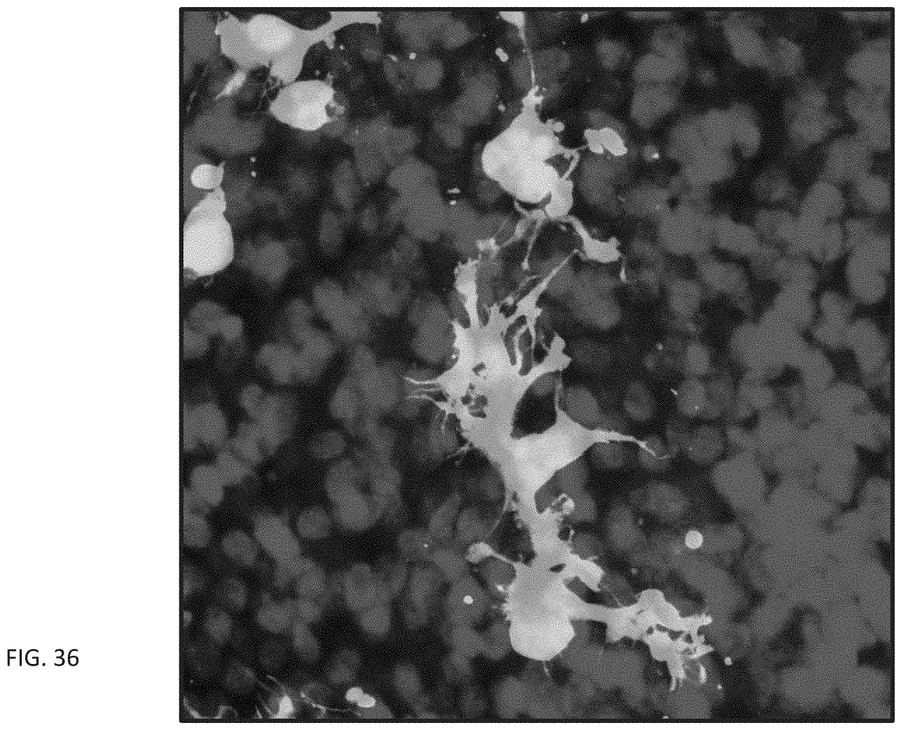

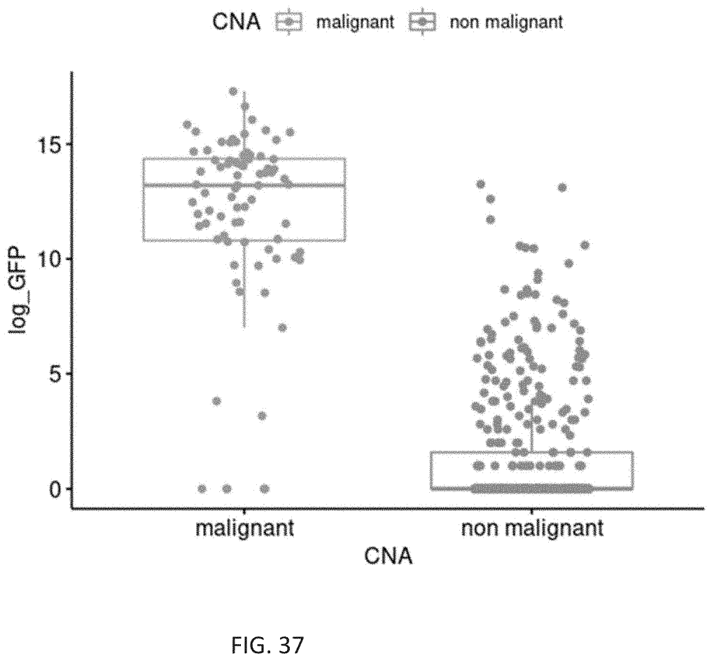

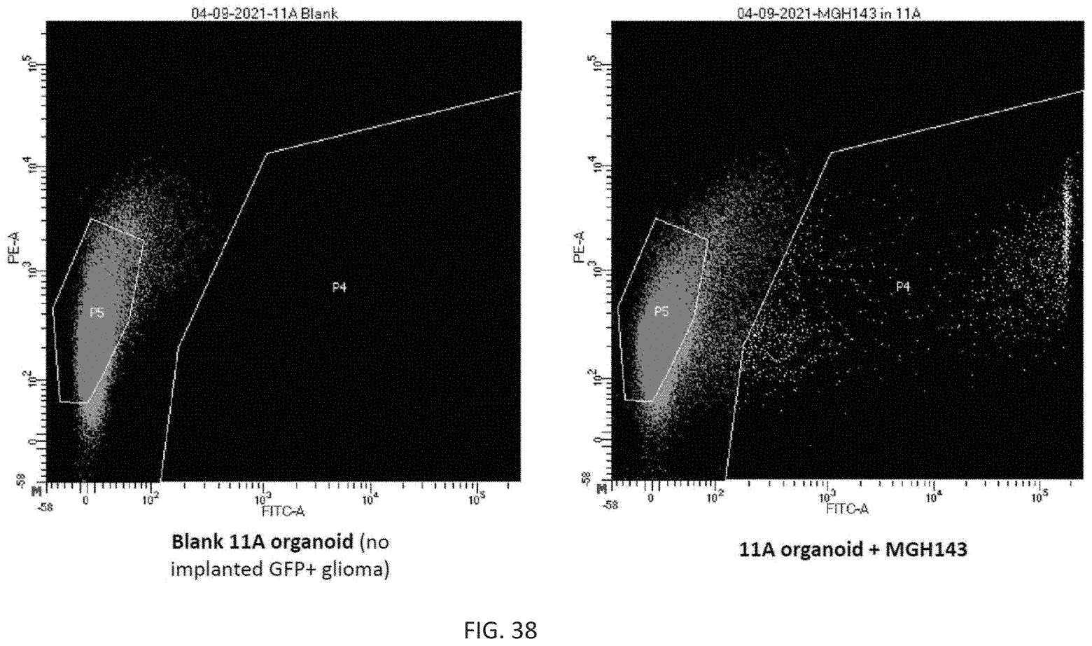

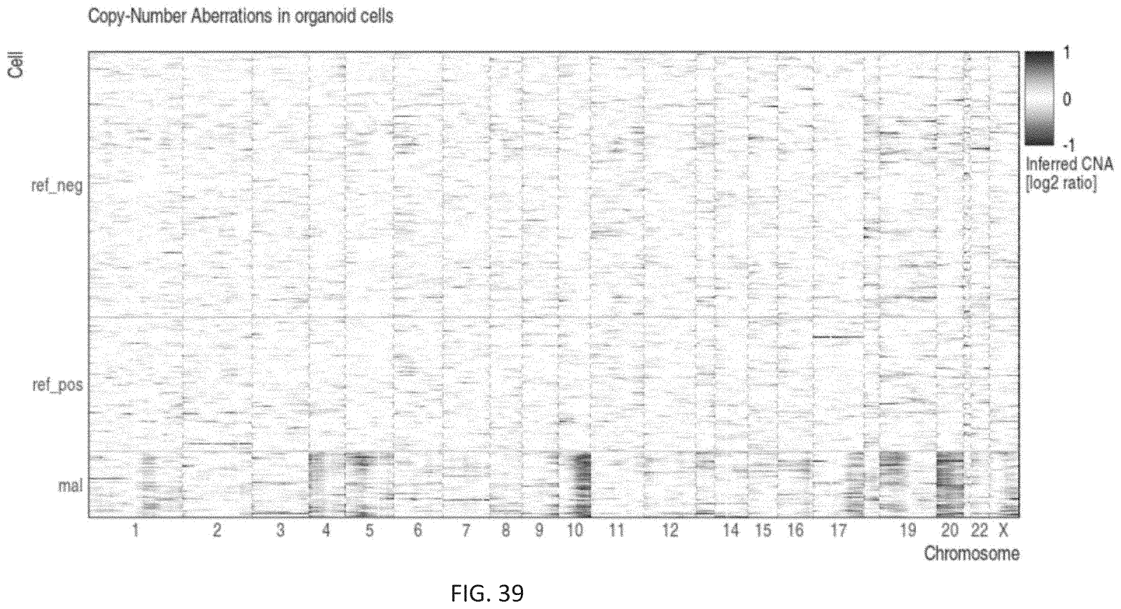

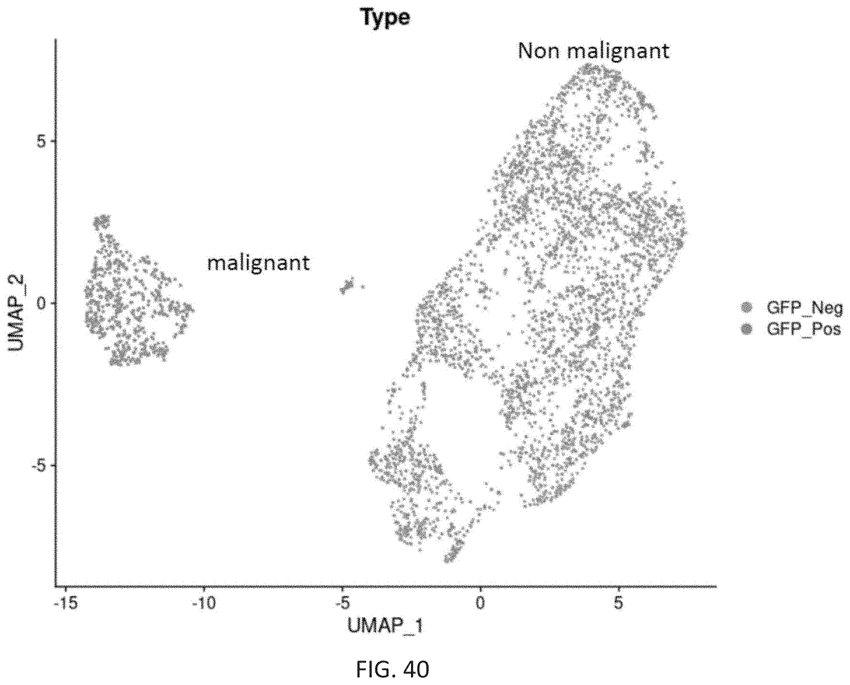

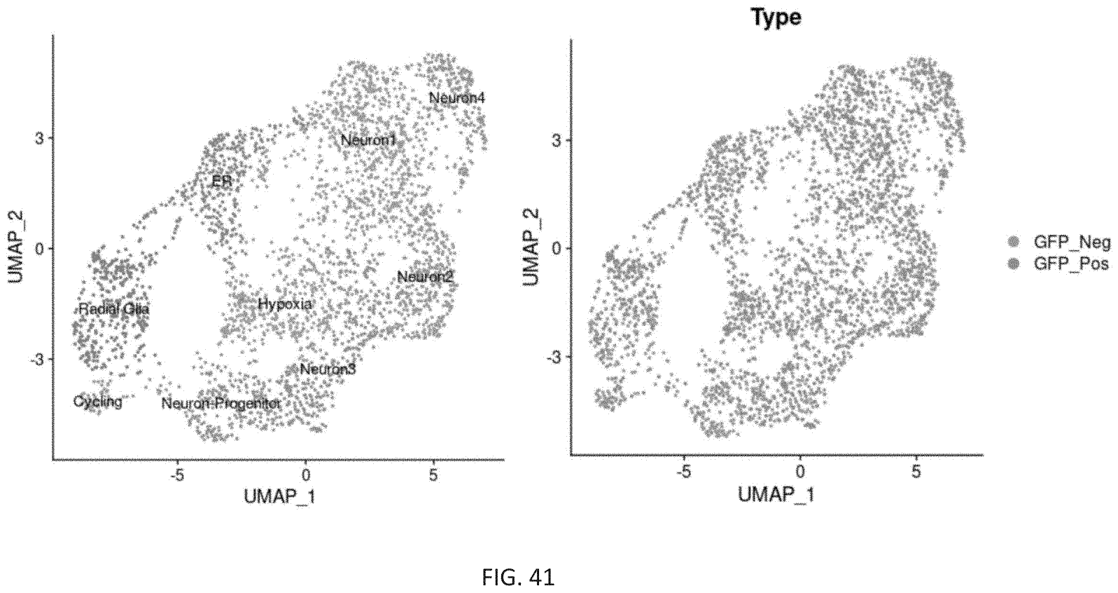

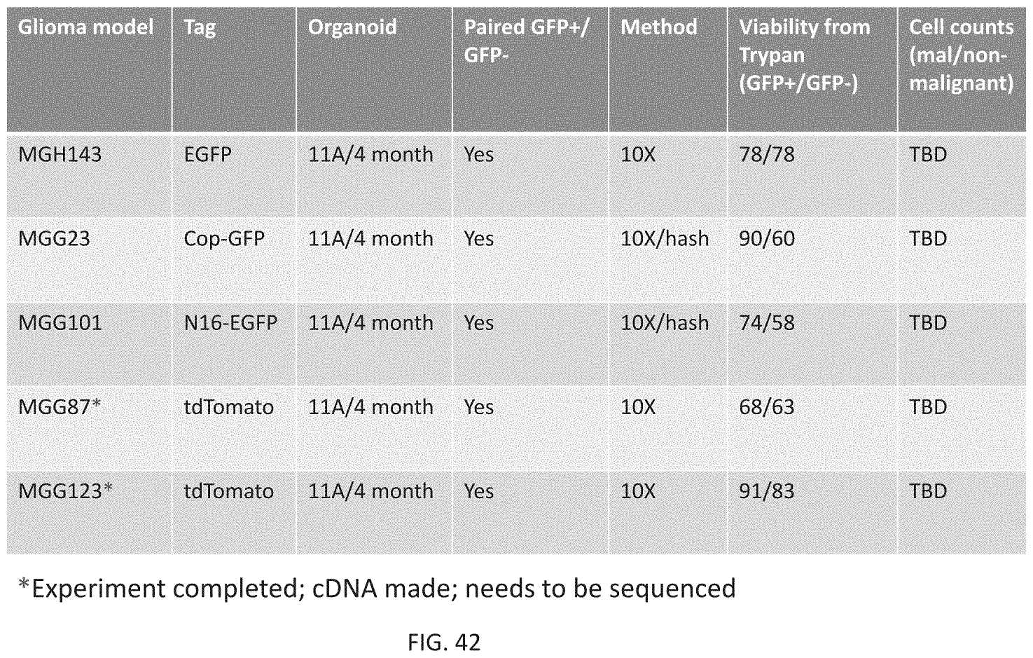

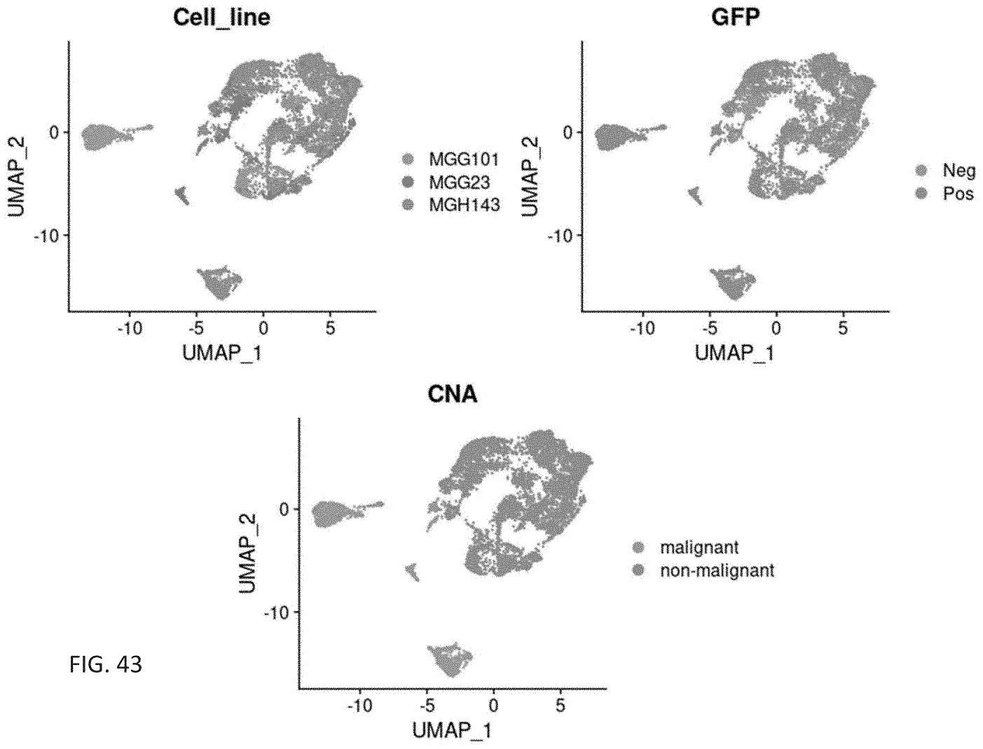

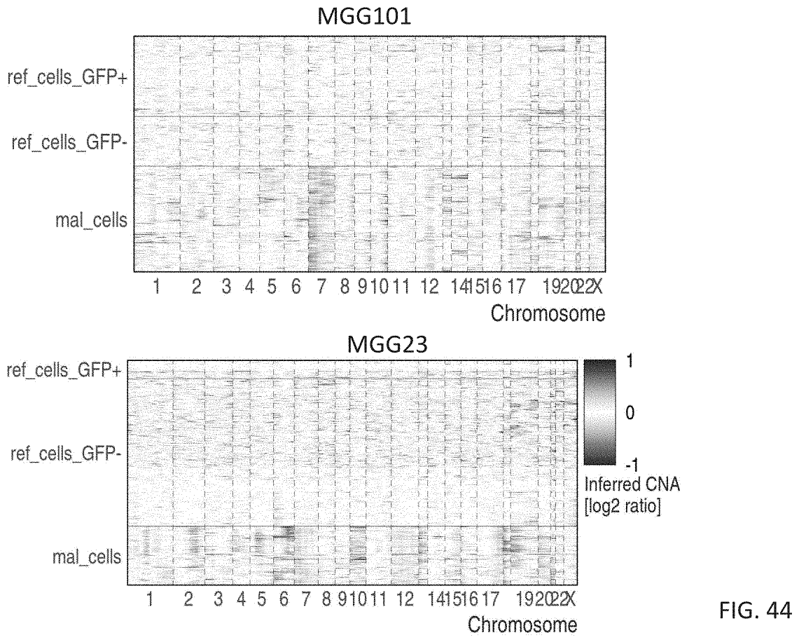

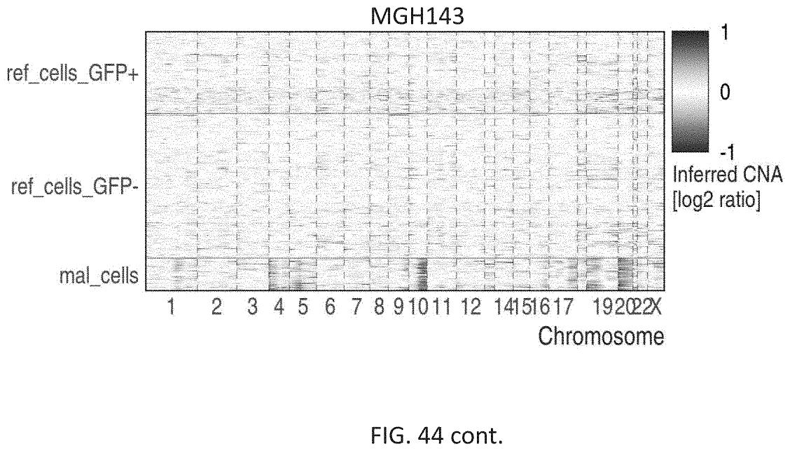

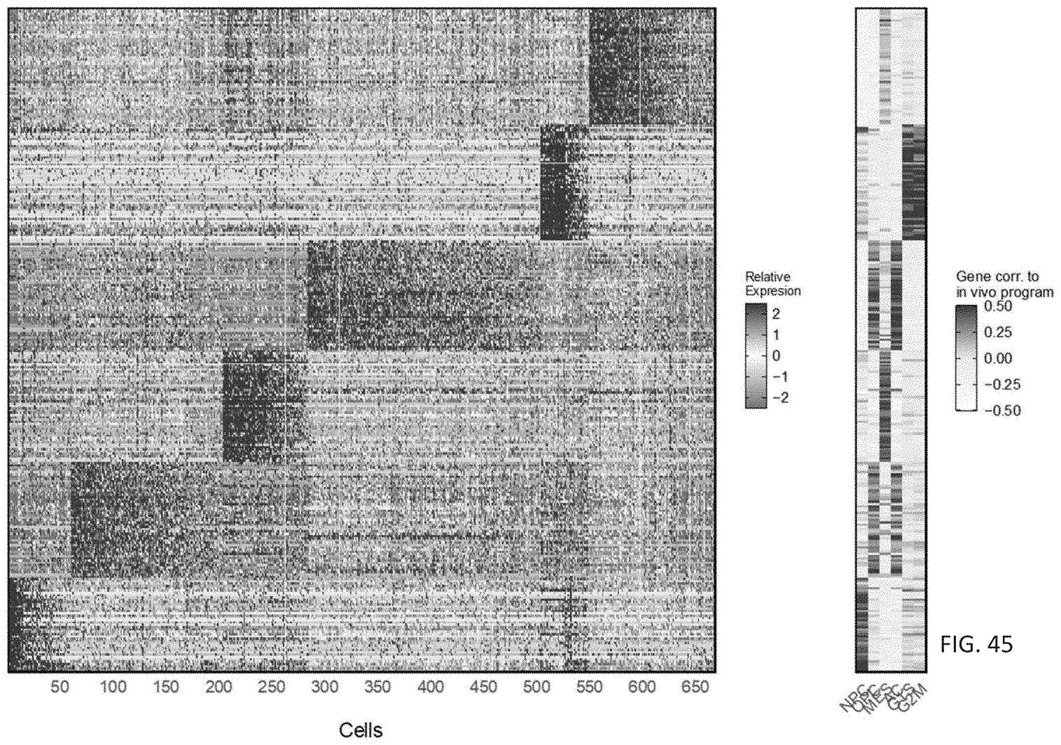

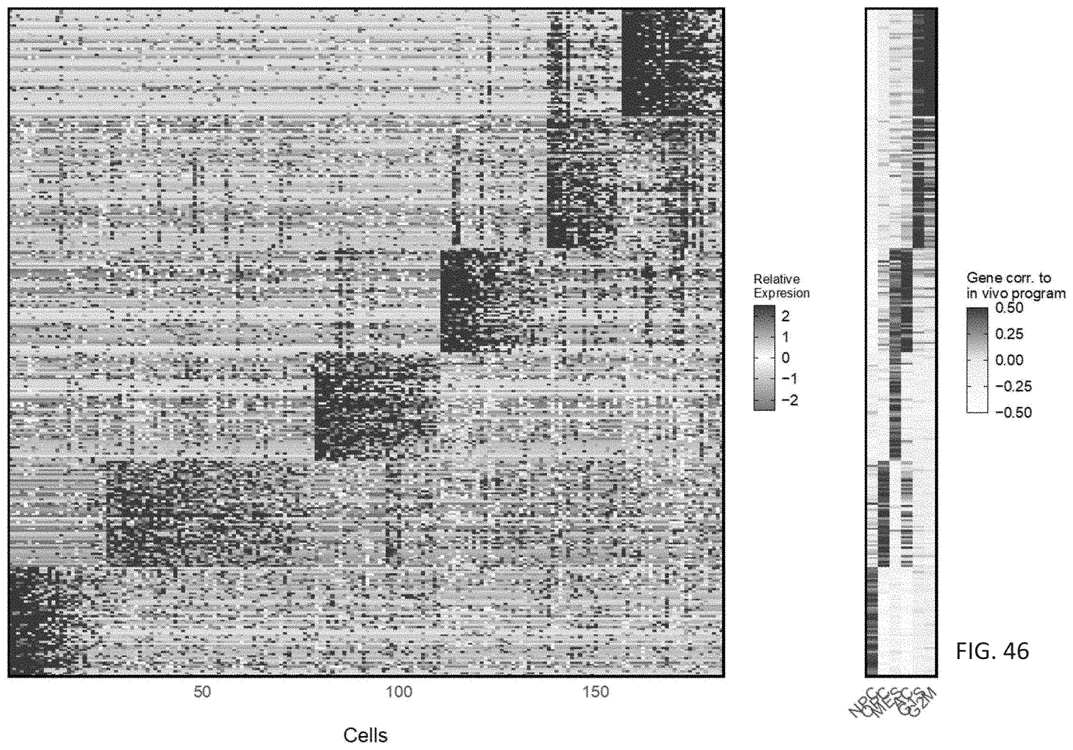

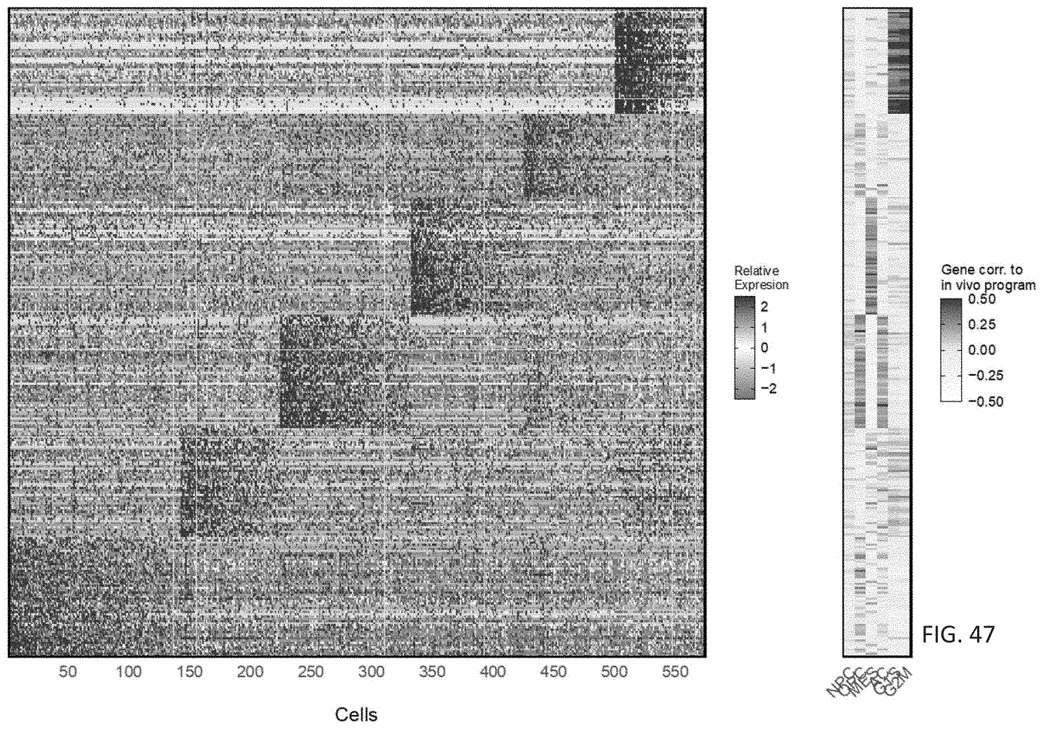

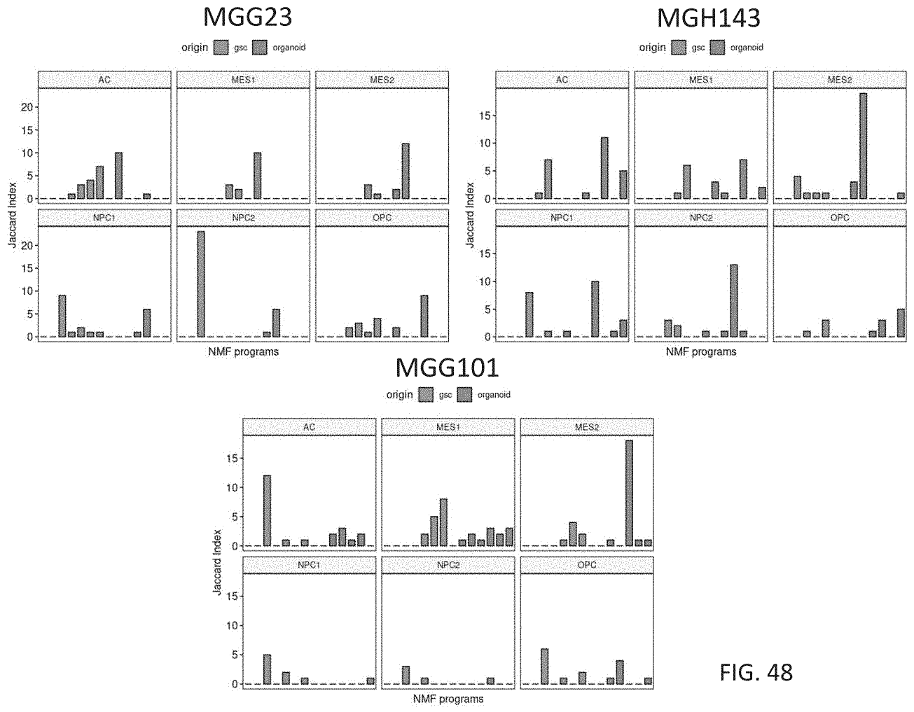

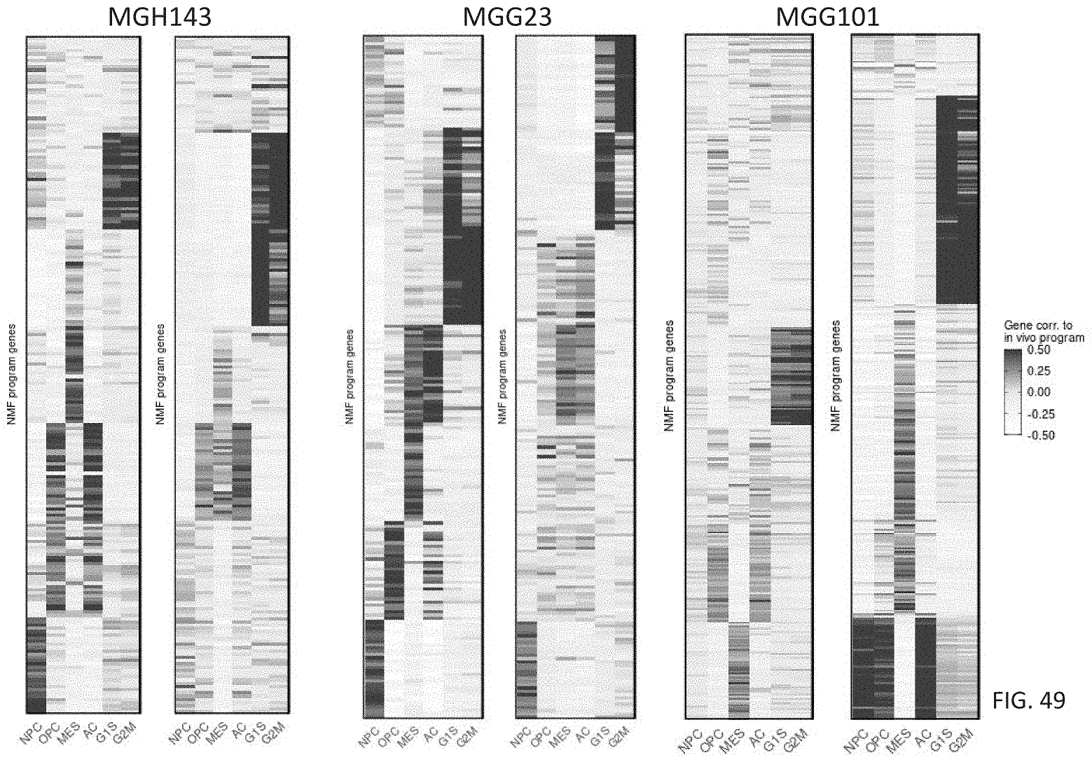

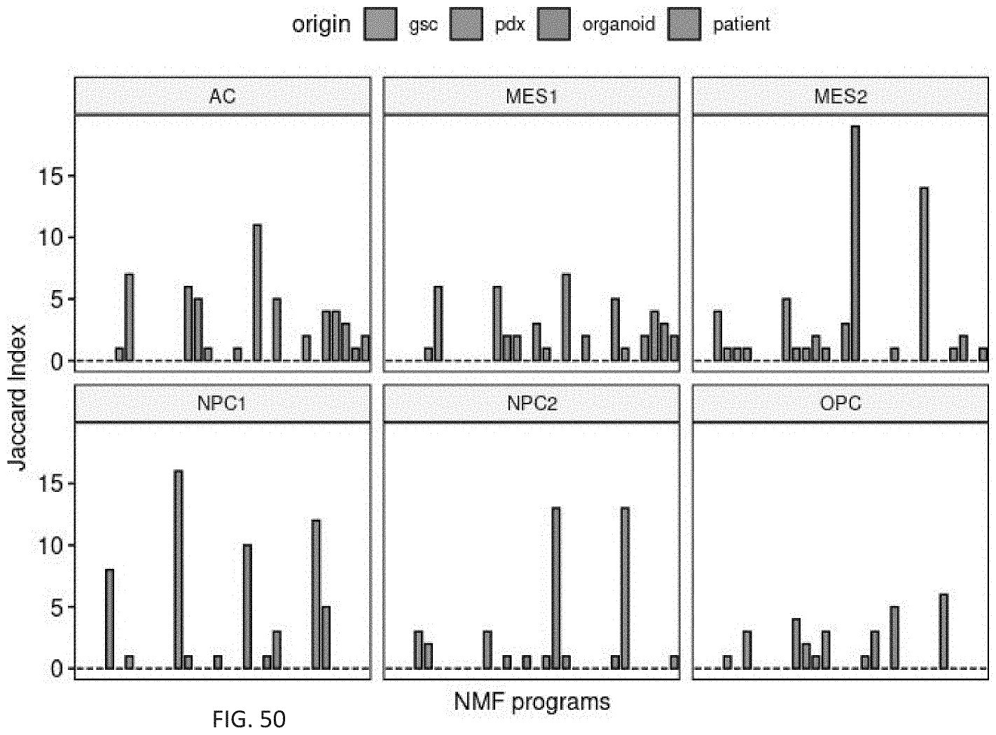

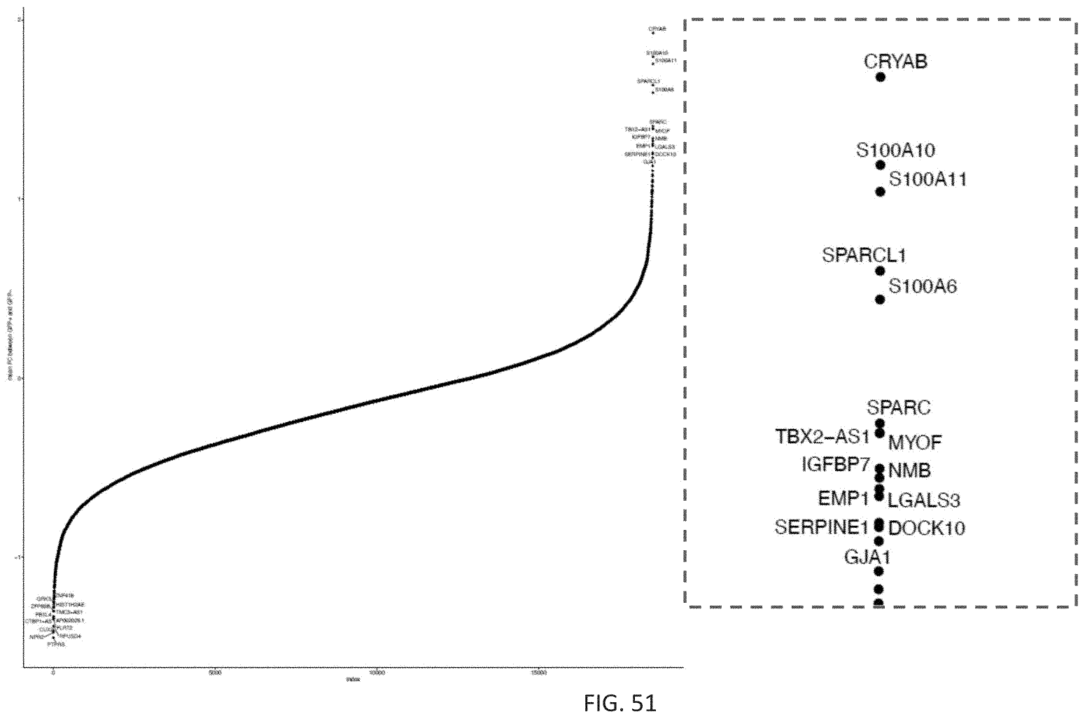

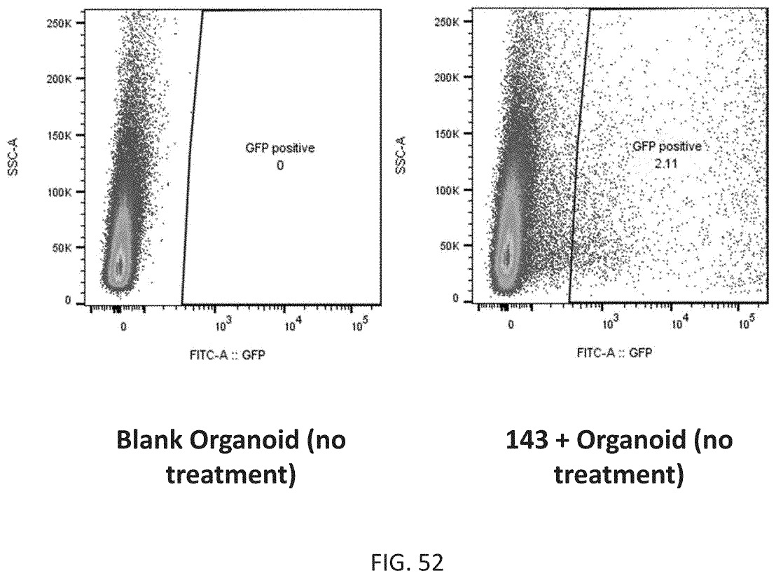

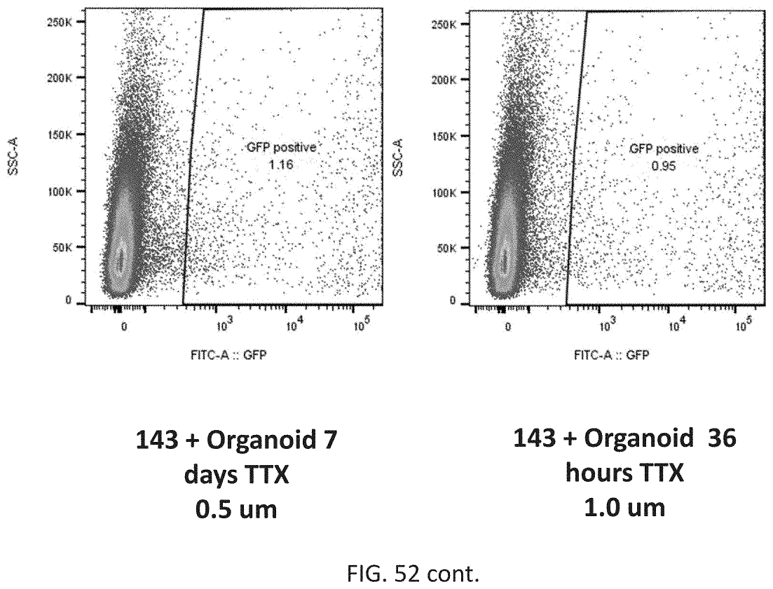

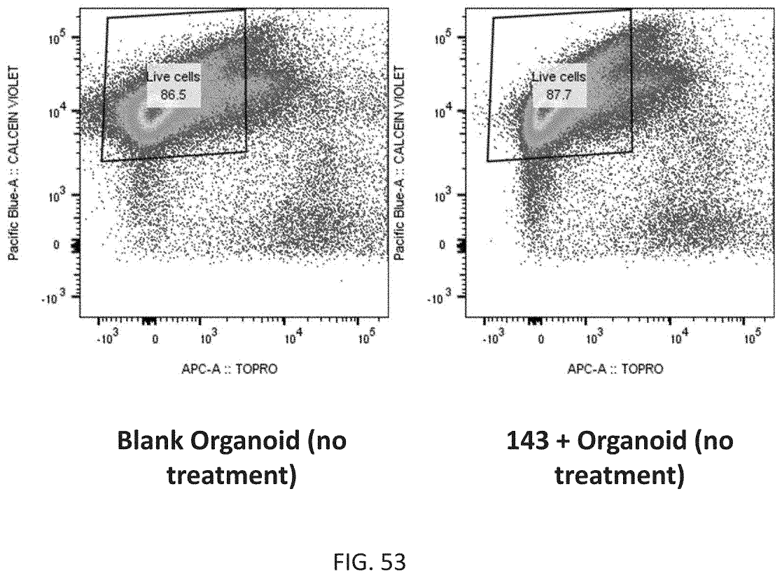

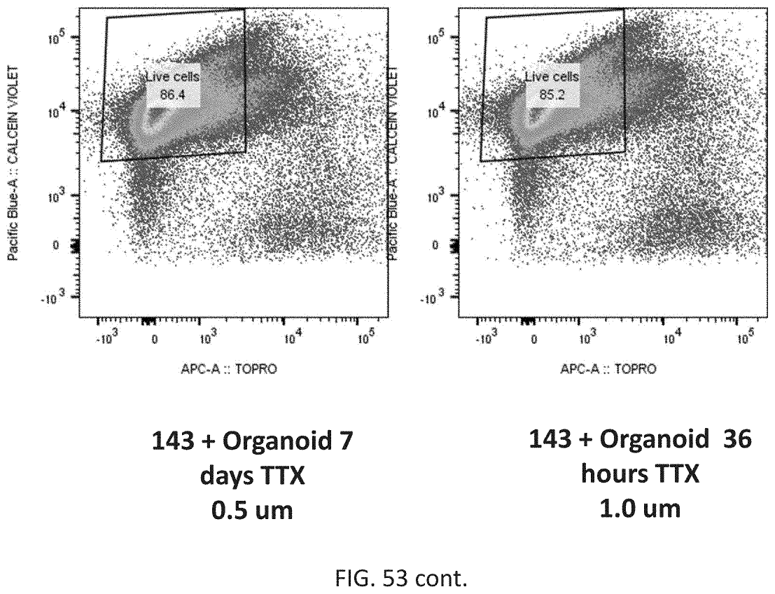

Using gliomaspheres, only 1 human glioblastoma (GBM) state was recreated (FIG. 29). Each cell was plotted based on its relative scoring amongst the four known GBM cell states. This data included 275 human cells and 74 cells of gliomaspheres cells. Using the organoid glioma model, at least 3 human GMB states were recreated (FIG. 30). 275 human GMB cells and 66 organoid glioma model cells were included. Using patient-derived glioma (PDX) cells, all 4 human GMB states were recreated (FIG. 31). This data included 275 human GMB cells and 75 PDX cells. Malignant cell scores across models for 8 gene signatures observed in human glioblastomas were shown in FIG. 32.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A composition comprising
a. a dorsal forebrain organoid having a core comprising less than 25% apoptotic or hypoxic cells; and
b. one or more brain tumor cells in the organoid.

2. The composition of claim 1, wherein the core comprises less than 20%, less than 15%, less than 10%, less than 5%, less than 1%, or less than 0.1% apoptotic or hypoxic cells.

3. The composition of claim 1, wherein the organoid has been cultured for at least 3 months.

4. The composition of claim 3, wherein the organoid comprises one or more of: corticofugal projection neurons, callosal projection neurons, cycling progenitors, immature corticofugal projection neurons, immature callosal projection neurons, immature projection neurons, immature interneurons, intermediate progenitor cells, outer radial glia, Cajal-Retzius neurons, and radial glia.

5. The composition of claim 4, wherein the organoid comprises:
a. about 17%-28% corticofugal projection neurons,
b. about 40%-50% callosal projection neurons,
c. about 4%-7% cycling progenitors,
d. about 2% or less immature interneurons,
e. about 3%-15% immature projection neurons,
f. about 3%-6% intermediate progenitor cells,
g. about 9%-14% radial glia,
h. about 0.5% or less of Cajal-Retzius neurons,
i. substantially no astroglia or cycling interneuron precursors, or
j. any combination thereof.

6. The composition of claim 1, wherein the organoid has been cultured for at least 6 months.

7. The composition of claim 6, wherein the organoid comprises one or more of: astroglia, callosal projection neurons, cycling progenitors, immature callosal projection neurons, immature interneurons, immature projection neurons, intermediate progenitor cells, outer radial glia, radial glia, ventral precursors, outer radial glia/astroglia, and cycling interneuron precursors.

8. The composition of claim 7, wherein the organoid comprises:
a. about 6%-16% astroglia,
b. about 7%-22% callosal projection neurons,
c. about 5%-8% cycling progenitors,
d. about 10%-31% immature interneurons,
e. about 2%-10% immature projection neurons,
f. about 1%-7% intermediate progenitor cells,
g. about 22%-39% radial glia,
h. about 4%-8% ventral precursors,
i. substantially no corticofugal projection neurons or immature corticofugal projection neurons, or
j. any combination thereof.

9. The composition of claim 1, wherein the organoid has been cultured for at least 9 months or at least a year.

10. The composition of claim 9, wherein the human patient-derived glioma cells comprise grade IV glioblastoma cells, high grade pediatric glioma cells, diffuse intrinsic pontine glioma (DIPG) cells, or isocitrate dehydrogenase (IDH) mutant glioma cells.

11. The composition of claim 9, wherein the human patient-derived glioma cells comprise IDH-wild type primary glioblastoma cells, IDH-mutant astrocytoma cells, or IDH-mutant oligodendroglioma cells.

12. The composition of claim 1, wherein the organoid is a human dorsal forebrain organoid.

13. The composition of claim 1, wherein the brain tumor cells comprise glioma cells.

14. The composition of claim 13, wherein the glioma cells comprise one or more, two or more, or three or more of: OPC-like cells, AC-like cells, NPC-like cells, or MES-like cells.

15. The composition of claim 13, wherein the glioma cells originate from human patient-derived glioma cells implanted into the organoid.

16. The composition of claim 13, wherein the glioma cells comprise glioblastoma cells.

17. The composition of claim 13, wherein the glioma cells and/or cells in the organoid express one or more reporter genes.

18. The composition of claim 1, wherein the composition comprises a ratio of malignant cells to non-malignant cells.

19. The composition of claim 1, wherein the brain tumor cells have been implanted into the organoid.

20. A method of modeling a brain tumor, the method comprising: implanting brain tumor cells into a dorsal forebrain organoid with a core comprising less than 25% apoptotic or hypoxic cells.

21. The method of claim 20, wherein the core comprises less than 20%, less than 15%, less than 10%, less than 5%, less than 1%, or less than 0.1% apoptotic or hypoxic cells.

22. The method of claim 20, wherein the organoid has been cultured for at least 3 months.

23. The method of claim 22, wherein the organoid comprises one or more of: corticofugal projection neurons, callosal projection neurons, cycling progenitors, immature corticofugal projection neurons, immature callosal projection neurons, immature projection neurons, immature interneurons, intermediate progenitor cells, outer radial glia, Cajal-Retzius neurons, and radial glia.

24. The method of claim 22, wherein the organoid comprises:
a. about 17%-28% corticofugal projection neurons,
b. about 40%-50% callosal projection neurons,
c. about 4%-7% cycling progenitors,
d. about 2% or less immature interneurons,
e. about 3%-15% immature projection neurons,
f. about 3%-6% intermediate progenitor cells,
g. about 9%-14% radial glia,
h. about 0.5% or less of Cajal-Retzius neurons,
i. substantially no astroglia or cycling interneuron precursors, or
j. any combination thereof.

25. The method of claim 20, wherein the organoid has been cultured for at least 6 months.

26. The method of claim 25, wherein the organoid comprises one or more of: astroglia, callosal projection neurons, cycling progenitors, immature callosal projection neurons, immature interneurons, immature projection neurons, intermediate progenitor cells, outer radial glia, radial glia, ventral precursors, outer radial glia/astroglia, and cycling interneuron precursors.

27. The method of claim 25, wherein the organoid comprises:
a. about 6%-16% astroglia,
b. about 7%-22% callosal projection neurons,
c. about 5%-8% cycling progenitors,
d. about 10%-31% immature interneurons,
e. about 2%-10% immature projection neurons,
f. about 1%-7% intermediate progenitor cells,
g. about 22%-39% radial glia,
h. about 4%-8% ventral precursors,
i. substantially no corticofugal projection neurons or immature corticofugal projection neurons, or
j. any combination thereof.

28. The method of claim 20, wherein the organoid has been cultured for at least 9 months or at least a year.

29. The method of claim 20, wherein the brain tumor is a glioma.

30. The method of claim 20, wherein the brain tumor cells are glioma cells.

31. The method of claim 30, wherein the glioma cells comprise glioblastoma cells.

32. The method of claim 30, wherein the glioma cells are patient-derived glioma cells.

33. The method of claim 32, wherein the patient-derived glioma cells grow to glioma cells comprising one or more of: OPC-like cells, AC-like cells, NPC-like cells, or MES-like cells.

34. The method of claim 32, wherein the patient-derived glioma cells grow to glioma cells comprising two or more, or three or more of OPC-like cells, AC-like cells, NPC-like cells, and MES-like cells.

35. The method of claim 32, wherein the patient-derived glioma cells comprise grade IV glioblastoma cells, high grade pediatric glioma cells, diffuse intrinsic pontine glioma (DIPG) cells, or isocitrate dehydrogenase (IDH) mutant glioma cells.

36. The method of claim 20, wherein the implantation is performed by seeding the brain tumor cells on a surface of the brain organoid.

37. The method of claim 20, further comprising testing growth rates, transcriptional states, cellular lineages and/or hierarchies, cell morphologies, tumor-organoid microenvironmental interactions, invasive potential of tumor cells, intercellular communication, and/or intercellular connectivity of the tumor cells.

38. A method of identifying genetic variations related to a brain tumor, the method comprising:
a. introducing one or more genetic variations to the composition of claim 1; and
b. testing effects of the one or more genetic variations on growth rates, transcriptional states, cellular lineages and/or hierarchies, cell morphologies, tumor-organoid microenvironmental interactions, invasive potential of tumor cells, intercellular communication, and/or intercellular connectivity of the tumor cells.

39. The method of claim 38, wherein the one more genetic variations is introduced into the brain tumor cells and the method comprises testing effect of the one or more genetic variations on cells in the organoid.

40. A method of screening a therapeutic agent, the method comprising:
a. contacting the composition of claim 1 with one or more candidate agents; and
b. testing effects of the one or more candidate agents on growth rates, transcriptional states, cellular lineages and/or hierarchies, cell morphologies,
c. tumor-organoid microenvironmental interactions, invasive potential of tumor cells, intercellular communication, and/or intercellular connectivity of the tumor cells.
